(19) United States
(12) Patent Application Publication
(10) Pub. No.: US 2010/0286092 A1
(43) Pub. Date: Nov. 11, 2010

(54) BETA-LACTAMASE INHIBITORS

(76) Inventors: Christopher J. Burns, Malvern, PA (US); Randy W. Jackson, Glenmoore, PA (US); Rajesh Goswami, Upper Darby, PA (US); Hongyu Xu, Phoenixville, PA (US)

Correspondence Address: NOVARTIS INSTITUTES FOR BIOMEDICAL RESEARCH, INC., 220 MASSACHUSETTS AVENUE, CAMBRIDGE, MA, 02139, US

(21) Appl. No.: 12/742,526

(22) PCT Filed: Nov. 13, 2008
(86) PCT No.: PCT/US08/12706

§ 371 Date: Aug. 2, 2010

Related U.S. Application Data
(60) Provisional application No. 61/002,797, filed on Nov. 13, 2007.

(51) Int. Cl. A61K 31/69; C07F 5/02; C07D 333/02; A61P 31/04

(52) U.S. Cl. 514/64; 546/13; 548/110; 549/4; 564/8; 568/6

(57) ABSTRACT

Disclosed herein are α-aminoboronic acids and their derivatives which act as inhibitors of beta-lactamases. Also disclosed herein are pharmaceutical compositions comprising α-aminoboronic acids and methods of use thereof.

### CROSS-REFERENCE TO RELATED APPLICATION

This application claims the benefit of U.S. Provisional Application No. 61/002,797, filed Nov. 13, 2007, which is incorporated by reference in its entirety.

### FIELD OF THE INVENTION

The present disclosure relates to α-aminoboronic acids and their derivatives which act as inhibitors of beta-lactamase enzymes.

### BACKGROUND OF THE INVENTION

Antibiotics are the most effective drugs for curing bacteria-infectious diseases clinically. They have a wide market for their advantages of good antibacterial effect, and limited side effect. Among them, beta-lactam antibiotics (for example, penicillins, cephalosporins, and carbapenems) are widely used because they have a very strong bactericidal effect (by blocking cell division) and very low toxicity.

To counter the efficacy of the various beta-lactams, bacteria have evolved to produce variants of beta-lactam deactivating enzymes called beta-lactamases, and in the ability to share this tool inter- and intra-species. The rapid spread of this mechanism of bacterial resistance can severely limit beta-lactam treatment options in the hospital and in the community. Beta-lactamases are typically grouped into 4 classes: Ambler classes A, B, C, and D, based on their amino acid sequences. Enzymes in classes A, C, and D are active-site serine beta-lactamases, while class B enzymes, which are encountered less frequently, are Zn-dependent. Newer generation cephalosporins and carbapenems were developed partly based on their ability to evade the deactivating effect of the early serine-based beta-lactamase variants. However, a recent surge in new versions of serine-based beta-lactamases—for example Class A Extended-Spectrum Beta-Lactamase (ESBL) enzymes, Class A carbapenemases (e.g. KPC-2), chromosomal and plasmid mediated Class C cephalosporinases (AmpC, CMY, etc.), and Class D oxacillinases—has begun to diminish the utility of the beta-lactam antibiotic family, including the more recent generation beta-lactam drugs, leading to a serious medical problem. Indeed the number of catalogued serine-based beta-lactamases has exploded from less than ten in the 1970s to over 300 variants (see, e.g., Jacoby & Bush, “Amino Acid Sequences for TEM, SHV and OXA Extended-Spectrum and Inhibitor Resistant β-Lactamases”, on the Lahey Clinic website).

The commercially available beta-lactamase inhibitors (clavulanic acid, sulbactam, tazobactam) were developed to address the beta-lactamases that were clinically relevant in the 1970s and 1980s (e.g. penicillinases). These enzyme inhibitors are available only as fixed combinations with penicillin derivatives. No combinations with cephalosporins (or carbapenems) have been developed or are clinically available. This fact, combined with the increased use of newer generation cephalosporins and carbapenems, is driving the selection and spread of the new beta-lactamase variants (ESBLs, carbapenemases, chromosomal and plasmid-mediated class C, class D oxacillinases, etc.). While maintaining good inhibitory activity against ESBLs, the legacy beta-lactamase inhibitors are largely ineffective against the new Class A carbapenemases, against the chromosomal and plasmid-mediated Class C cephalosporinases and against many of the Class D oxacillinases. To address this growing therapeutic vulnerability, a new generation of beta-lactamase inhibitors must be developed with broad spectrum functionality. The novel boronic acid based inhibitors described herein address this medical need.

Use of a boronic acid compound to inhibit a beta-lactamase enzyme has been limited. For example, U.S. Pat. No. 7,271,186 discloses beta-lactamase inhibitors that target AmpC (from class C). Ness et al. (Biochemistry (2000) 39:5312-21) discloses beta-lactamase inhibitors that target TEM-1 (a non-ESBL TEM variant from class A; one of approximately 140 known TEM-type beta-lactamase variants). Because there are three major molecular classes of serine-based beta-lactamases, and each of these classes contain significant numbers of beta-lactamase variants, inhibition of one or a small number of beta-lactamases is unlikely to be of therapeutic value. Therefore, there is an imperative need to develop novel beta-lactamase inhibitors with broad spectrum functionality.

### SUMMARY OF THE INVENTION

One aspect is for a compound of the formula:

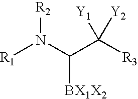

wherein R₁ is —C(O)R₄; —C(O)NR₄R₅; —C(O)OR₄; —S(O)₂R₄, —C(═NR₄R₅)R₄, —C(═NR₄R₅)NR₄R₅, hydrogen, or is selected from the group consisting of:
(a) aryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido,
(b) heteroaryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido, and
(c) heterocyclic group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido;
   R₂ is hydrogen, or is selected from the group consisting of:
(a) C1-C6 alkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the C1-C6 carbons comprise part of said oxyimino group, sulfido, and sulfoxido,
(b) C3-C7 cycloalkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the cycloalkyl group other than the one attached to the rest of the molecule comprise part of said oxyimino group, sulfido, and sulfoxido,
(c) aryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido,
(d) heteroaryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido, and
(e) heterocyclic group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, sulfido, and sulfoxido;
   R₃ is an aryl or heteroaryl group substituted with from 1 to 4 substituents wherein one of the substituents is a hydroxyl or amino group located at the 2 position relative to the group containing Y₁ and Y₂, and wherein the remaining substituents are selected from the group consisting of hydroxyl, alkyl, cycloalkyl, alkoxy, alkenyl, alkynyl, amino, aminocarbonyl, carbonyl, aminosulfonyl, alkylaryl, aryl, aryloxy, carboxyl, cyano, guanidino, halogen, heteroaryl, heterocyclyl, sulfido, sulfonyl, sulfoxido, sulfonic acid, sulfate, and thiol;
   R₄ is selected from the group consisting of:
(a) C1-C10 alkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the C1-C10 carbons comprise part of said oxyimino group, sulfido, and sulfoxido,
(b) C3-C10 cycloalkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the cycloalkyl group other than the one attached to the rest of the molecule comprise part of said oxyimino group, sulfido, and sulfoxido,
(c) aryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido,
(d) heteroaryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido, and
(e) heterocyclic group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, sulfido, and sulfoxido;
   R₅ is hydrogen or is selected from the group consisting of:
(a) C1-C6 alkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the C1-C10 carbons comprise part of said oxyimino group, sulfido, and sulfoxido,
(b) C3-C7 cycloalkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the cycloalkyl group other than the one attached to the rest of the molecule comprise part of said oxyimino group, sulfido, and sulfoxido,
(c) aryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido,
(d) heteroaryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido, and
(e) heterocyclic group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, sulfido, and sulfoxido;
   X₁ and X₂ are independently hydroxyl, halogen, NR₄R₅, C1-C6 alkoxy, or when taken together X₁ and X₂ form a cyclic boron ester where said chain or ring contains from 2 to 20 carbon atoms and, optionally, 1-3 heteroatoms which can be O, N, or S, or when taken together X₁ and X₂ form a cyclic boron amide where said chain or ring contains from 2 to 20 carbon atoms and, optionally, 1-3 heteroatoms which can be O, N, or S, or when taken together X₁ and X₂ form a cyclic boron amide-ester where said chain contains from 2-20 carbon atoms and, optionally, 1-3 heteroatoms which can be O, N, or S, or X₁ and R₁ together form a cyclic ring where said ring contains 2 to 10 carbon atoms and, optionally, 1-3 heteroatoms which can be O, N, or S, and X₂ is hydroxyl, halogen, NR₄R₅, C1-C6 alkoxy, or X₁ and R₃ together form a cyclic ring where said ring contains 3 to 10 carbon atoms and, optionally, 1-3 heteroatoms which can be O, N, or S, and X₂ is hydroxyl, halogen, NR₄R₅, or C1-C6 alkoxy;
   Y₁ and Y₂ are independently hydrogen, alkyl, cycloalkyl, alkoxy, alkenyl, alkynyl, amino, aminosulfonyl, aminocarbonyl, carbonyl, alkylaryl, aryl, aryloxy, carboxyl, cyano, halogen, heteroaryl, heteroaryloxy, heterocyclyl, sulfido, sulfonyl, or sulfoxido, or taken together Y₁ and Y₂ form a cyclic structure containing from 3-12 carbon atoms and, optionally, 1-3 heteroatoms which can be O, N, or S; or a salt thereof;
   provided that, when R₁ is —C(O)R₄, R₂ is hydrogen, R₃ is a phenyl group having two substituents consisting of a hydroxyl at the 2-position and a carboxylic acid at the 3-position relative to the group containing Y₁ and Y₂, X₁ and X₂ are hydroxyl or X₁ is hydroxyl and X₂ is replaced by the ortho-hydroxyl oxygen of R₃ such that a 6-membered ring is formed, and Y₁ and Y₂ are hydrogen, R₄ is not unsubstituted C1 alkyl.

Another aspect is for a pharmaceutical composition comprising: (a) one or more compounds discussed above; (b) one or more β-lactam antibiotics; and (c) one or more pharmaceutically acceptable carriers.

A further aspect is for a pharmaceutical composition comprising: (a) one or more compounds discussed above; and (b) one or more pharmaceutically acceptable carriers.

An additional aspect is for a method of treating a bacterial infection in a mammal comprising administering to a mammal in need thereof:

(i) an effective amount of a compound having the formula:

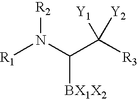

wherein R₁ is —C(O)R₄; —C(O)NR₄R₅; —C(O)OR₄; —S(O)₂R₄, —C(═NR₄R₅)R₄, —C(═NR₄R₅)NR₄R₅, hydrogen, or is selected from the group consisting of:
(a) aryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido,
(b) heteroaryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido, and
(c) heterocyclic group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido;
R₂ is hydrogen, or is selected from the group consisting of:
(a) C1-C6 alkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the C1-C6 carbons comprise part of said oxyimino group, sulfido, and sulfoxido,
(b) C3-C7 cycloalkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the cycloalkyl group other than the one attached to the rest of the molecule comprise part of said oxyimino group, sulfido, and sulfoxido,
(c) aryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido,
(d) heteroaryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido, and
(e) heterocyclic group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, sulfido, and sulfoxido;
R₃ is an aryl or heteroaryl group substituted with from 1 to 4 substituents wherein one of the substituents is a hydroxyl or amino group located at the 2 position relative to the group containing Y₁ and Y₂, and wherein the remaining substituents are selected from the group consisting of hydroxyl, alkyl, cycloalkyl, alkoxy, alkenyl, alkynyl, amino, aminocarbonyl, carbonyl, aminosulfonyl, alkylaryl, aryl, aryloxy, carboxyl, cyano, guanidino, halogen, heteroaryl, heterocyclyl, sulfido, sulfonyl, sulfoxido, sulfonic acid, sulfate, and thiol;
R₄ is selected from the group consisting of:
(a) C1-C10 alkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the C1-C10 carbons comprise part of said oxyimino group, sulfido, and sulfoxido,
(b) C3-C10 cycloalkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the cycloalkyl group other than the one attached to the rest of the molecule comprise part of said oxyimino group, sulfido, and sulfoxido,
(c) aryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido,
(d) heteroaryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido, and
(e) heterocyclic group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, sulfido, and sulfoxido;
R₅ is hydrogen or is selected from the group consisting of:
(a) C1-C6 alkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the C1-C10 carbons comprise part of said oxyimino group, sulfido, and sulfoxido,
(b) C3-C7 cycloalkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the cycloalkyl group other than the one attached to the rest of the molecule comprise part of said oxyimino group, sulfido, and sulfoxido,
(c) aryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido,
(d) heteroaryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido, and
(e) heterocyclic group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, sulfido, and sulfoxido;
X₁ and X₂ are independently hydroxyl, halogen, NR₄R₅, C1-C6 alkoxy, or when taken together X₁ and X₂ form a cyclic boron ester where said chain or ring contains from 2 to 20 carbon atoms and, optionally, 1-3 heteroatoms which can be O, N, or S, or when taken together X₁ and X₂ form a cyclic boron amide where said chain or ring contains from 2 to 20 carbon atoms and, optionally, 1-3 heteroatoms which can be O, N, or S, or when taken together X₁ and X₂ form a cyclic boron amide-ester where said chain contains from 2-20 carbon atoms and, optionally, 1-3 heteroatoms which can be O, N, or S, or X₁ and R₁ together form a cyclic ring where said ring contains 2 to 10 carbon atoms and, optionally, 1-3 heteroatoms which can be O, N, or S, and X₂ is hydroxyl, halogen, NR₄R₅, C1-C6 alkoxy, or X₁ and R₃ together form a cyclic ring where said ring contains 3 to 10 carbon atoms and, optionally, 1-3 heteroatoms which can be O, N, or S, and X₂ is hydroxyl, halogen, NR₄R₅, or C1-C6 alkoxy;
Y₁ and Y₂ are independently hydrogen, alkyl, cycloalkyl, alkoxy, alkenyl, alkynyl, amino, aminosulfonyl, aminocarbonyl, carbonyl, alkylaryl, aryl, aryloxy, carboxyl, cyano, halogen, heteroaryl, heteroaryloxy, heterocyclyl, sulfido, sulfonyl, or sulfoxido, or taken together Y₁ and Y₂ form a cyclic structure containing from 3-12 carbon atoms and, optionally, 1-3 heteroatoms which can be O, N, or S;
or a salt thereof; and

(ii) an effective amount of a β-lactam antibiotic.

Another aspect is for a method of treating a bacterial infection in a mammal comprising administering to a mammal in need thereof an effective amount of a compound having the formula:

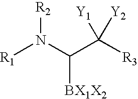

wherein R₁ is —C(O)R₄; —C(O)NR₄R₅; —C(O)OR₄; —S(O)₂R₄, —C(═NR₄R₅)R₄, —C(═NR₄R₅)NR₄R₅, hydrogen, or is selected from the group consisting of:
(a) aryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido,
(b) heteroaryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido, and
(c) heterocyclic group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido;
   R₂ is hydrogen, or is selected from the group consisting of:
(a) C1-C6 alkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the C1-C6 carbons comprise part of said oxyimino group, sulfido, and sulfoxido,
(b) C3-C7 cycloalkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the cycloalkyl group other than the one attached to the rest of the molecule comprise part of said oxyimino group, sulfido, and sulfoxido,
(c) aryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido,
(d) heteroaryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido, and
(e) heterocyclic group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, sulfido, and sulfoxido;
   R₃ is an aryl or heteroaryl group substituted with from 1 to 4 substituents wherein one of the substituents is a hydroxyl or amino group located at the 2 position relative to the group containing Y₁ and Y₂, and wherein the remaining substituents are selected from the group consisting of hydroxyl, alkyl, cycloalkyl, alkoxy, alkenyl, alkynyl, amino, aminocarbonyl, carbonyl, aminosulfonyl, alkylaryl, aryl, aryloxy, carboxyl, cyano, guanidino, halogen, heteroaryl, heterocyclyl, sulfido, sulfonyl, sulfoxido, sulfonic acid, sulfate, and thiol;
   R₄ is selected from the group consisting of:
(a) C1-C10 alkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the C1-C10 carbons comprise part of said oxyimino group, sulfido, and sulfoxido,
(b) C3-C10 cycloalkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the cycloalkyl group other than the one attached to the rest of the molecule comprise part of said oxyimino group, sulfido, and sulfoxido,
(c) aryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido,
(d) heteroaryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido, and
(e) heterocyclic group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, sulfido, and sulfoxido;
   R₅ is hydrogen or is selected from the group consisting of:
(a) C1-C6 alkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the C1-C10 carbons comprise part of said oxyimino group, sulfido, and sulfoxido,
(b) C3-C7 cycloalkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the cycloalkyl group other than the one attached to the rest of the molecule comprise part of said oxyimino group, sulfido, and sulfoxido,
(c) aryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido,
(d) heteroaryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido, and
(e) heterocyclic group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, sulfido, and sulfoxido;
   X₁ and X₂ are independently hydroxyl, halogen, NR₄R₅, C1-C6 alkoxy, or when taken together X₁ and X₂ form a cyclic boron ester where said chain or ring contains from 2 to 20 carbon atoms and, optionally, 1-3 heteroatoms which can be O, N, or S, or when taken together X₁ and X₂ form a cyclic boron amide where said chain or ring contains from 2 to 20 carbon atoms and, optionally, 1-3 heteroatoms which can be O, N, or S, or when taken together X₁ and X₂ form a cyclic boron amide-ester where said chain contains from 2-20 carbon atoms and, optionally, 1-3 heteroatoms which can be O, N, or S, or X₁ and R₁ together form a cyclic ring where said ring contains 2 to 10 carbon atoms and, optionally, 1-3 heteroatoms which can be O, N, or S, and X₂ is hydroxyl, halogen, NR₄R₅, C1-C6 alkoxy, or X₁ and R₃ together form a cyclic ring where said ring contains 3 to 10 carbon atoms and, optionally, 1-3 heteroatoms which can be O, N, or S, and X₂ is hydroxyl, halogen, NR₄R₅, or C1-C6 alkoxy;
   Y₁ and Y₂ are independently hydrogen, alkyl, cycloalkyl, alkoxy, alkenyl, alkynyl, amino, aminosulfonyl, aminocarbonyl, carbonyl, alkylaryl, aryl, aryloxy, carboxyl, cyano, halogen, heteroaryl, heteroaryloxy, heterocyclyl, sulfido, sulfonyl, or sulfoxido, or taken together Y₁ and Y₂ form a cyclic structure containing from 3-12 carbon atoms and, optionally, 1-3 heteroatoms which can be O, N, or S;
   or a salt thereof.

A further aspect is for a method of reducing bacterial resistance to a β-lactam antibiotic comprising contacting a bacterial cell having resistance to a β-lactam antibiotic with an effective amount of a beta-lactamase inhibitor with broad-spectrum functionality having the formula:

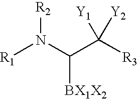

wherein R₁ is —C(O)R₄; —C(O)NR₄R₅; —C(O)OR₄; —S(O)₂R₄, —C(═NR₄R₅)R₄, —C(═NR₄R₅)NR₄R₅, hydrogen, or is selected from the group consisting of:
(a) aryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido,
(b) heteroaryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido, and
(c) heterocyclic group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido;
   R₂ is hydrogen, or is selected from the group consisting of:
(a) C1-C6 alkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the C1-C6 carbons comprise part of said oxyimino group, sulfido, and sulfoxido,
(b) C3-C7 cycloalkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the cycloalkyl group other than the one attached to the rest of the molecule comprise part of said oxyimino group, sulfido, and sulfoxido,
(c) aryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido,
(d) heteroaryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido, and
(e) heterocyclic group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, sulfido, and sulfoxido;
   R₃ is an aryl or heteroaryl group substituted with from 1 to 4 substituents wherein one of the substituents is a hydroxyl or amino group located at the 2 position relative to the group containing Y₁ and Y₂, and wherein the remaining substituents are selected from the group consisting of hydroxyl, alkyl, cycloalkyl, alkoxy, alkenyl, alkynyl, amino, aminocarbonyl, carbonyl, aminosulfonyl, alkylaryl, aryl, aryloxy, carboxyl, cyano, guanidino, halogen, heteroaryl, heterocyclyl, sulfido, sulfonyl, sulfoxido, sulfonic acid, sulfate, and thiol;
   R₄ is selected from the group consisting of:
(a) C1-C10 alkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the C1-C10 carbons comprise part of said oxyimino group, sulfido, and sulfoxido,
(b) C3-C10 cycloalkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the cycloalkyl group other than the one attached to the rest of the molecule comprise part of said oxyimino group, sulfido, and sulfoxido,
(c) aryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido,
(d) heteroaryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido, and
(e) heterocyclic group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, sulfido, and sulfoxido;
   R₅ is hydrogen or is selected from the group consisting of:
(a) C1-C6 alkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the C1-C10 carbons comprise part of said oxyimino group, sulfido, and sulfoxido,
(b) C3-C7 cycloalkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the cycloalkyl group other than the one attached to the rest of the molecule comprise part of said oxyimino group, sulfido, and sulfoxido,
(c) aryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido,
(d) heteroaryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido, and
(e) heterocyclic group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, sulfido, and sulfoxido;
   X₁ and X₂ are independently hydroxyl, halogen, NR₄R₅, C1-C6 alkoxy, or when taken together X₁ and X₂ form a cyclic boron ester where said chain or ring contains from 2 to 20 carbon atoms and, optionally, 1-3 heteroatoms which can be O, N, or S, or when taken together X₁ and X₂ form a cyclic boron amide where said chain or ring contains from 2 to 20 carbon atoms and, optionally, 1-3 heteroatoms which can be O, N, or S, or when taken together X₁ and X₂ form a cyclic boron amide-ester where said chain contains from 2-20 carbon atoms and, optionally, 1-3 heteroatoms which can be O, N, or S, or X₁ and R₁ together form a cyclic ring where said ring contains 2 to 10 carbon atoms and, optionally, 1-3 heteroatoms which can be O, N, or S, and X₂ is hydroxyl, halogen, NR₄R₅, C1-C6 alkoxy, or X₁ and R₃ together form a cyclic ring where said ring contains 3 to 10 carbon atoms and, optionally, 1-3 heteroatoms which can be O, N, or S, and X₂ is hydroxyl, halogen, NR₄R₅, or C1-C6 alkoxy;
   Y₁ and Y₂ are independently hydrogen, alkyl, cycloalkyl, alkoxy, alkenyl, alkynyl, amino, aminosulfonyl, aminocarbonyl, carbonyl, alkylaryl, aryl, aryloxy, carboxyl, cyano, halogen, heteroaryl, heteroaryloxy, heterocyclyl, sulfido, sulfonyl, or sulfoxido, or taken together Y₁ and Y₂ form a cyclic structure containing from 3-12 carbon atoms and, optionally, 1-3 heteroatoms which can be O, N, or S;
   or a salt thereof.

An additional aspect is for use of a beta-lactamase inhibitor with broad-spectrum functionality having the formula:

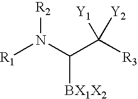

wherein R₁ is —C(O)R₄; —C(O)NR₄R₅; —C(O)OR₄; —S(O)₂R₄, —C(═NR₄R₅)R₄, —C(═NR₄R₅)NR₄R₅, hydrogen, or is selected from the group consisting of:
(a) aryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido,
(b) heteroaryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido, and
(c) heterocyclic group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido;
   R₂ is hydrogen, or is selected from the group consisting of:
(a) C1-C6 alkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the C1-C6 carbons comprise part of said oxyimino group, sulfido, and sulfoxido,
(b) C3-C7 cycloalkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the cycloalkyl group other than the one attached to the rest of the molecule comprise part of said oxyimino group, sulfido, and sulfoxido,
(c) aryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido,
(d) heteroaryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido, and
(e) heterocyclic group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, sulfido, and sulfoxido;
   R₃ is an aryl or heteroaryl group substituted with from 1 to 4 substituents wherein one of the substituents is a hydroxyl or amino group located at the 2 position relative to the group containing Y₁ and Y₂, and wherein the remaining substituents are selected from the group consisting of hydroxyl, alkyl, cycloalkyl, alkoxy, alkenyl, alkynyl, amino, aminocarbonyl, carbonyl, aminosulfonyl, alkylaryl, aryl, aryloxy, carboxyl, cyano, guanidino, halogen, heteroaryl, heterocyclyl, sulfido, sulfonyl, sulfoxido, sulfonic acid, sulfate, and thiol;
   R₄ is selected from the group consisting of:
(a) C1-C10 alkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the C1-C10 carbons comprise part of said oxyimino group, sulfido, and sulfoxido,
(b) C3-C10 cycloalkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the cycloalkyl group other than the one attached to the rest of the molecule comprise part of said oxyimino group, sulfido, and sulfoxido,
(c) aryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido,
(d) heteroaryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido, and
(e) heterocyclic group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, sulfido, and sulfoxido;
   R₅ is hydrogen or is selected from the group consisting of:
(a) C1-C6 alkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the C1-C10 carbons comprise part of said oxyimino group, sulfido, and sulfoxido,
(b) C3-C7 cycloalkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the cycloalkyl group other than the one attached to the rest of the molecule comprise part of said oxyimino group, sulfido, and sulfoxido,
(c) aryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido,
(d) heteroaryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido, and
(e) heterocyclic group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, sulfido, and sulfoxido;
   X₁ and X₂ are independently hydroxyl, halogen, NR₄R₅, C1-C6 alkoxy, or when taken together X₁ and X₂ form a cyclic boron ester where said chain or ring contains from 2 to 20 carbon atoms and, optionally, 1-3 heteroatoms which can be O, N, or S, or when taken together X₁ and X₂ form a cyclic boron amide where said chain or ring contains from 2 to 20 carbon atoms and, optionally, 1-3 heteroatoms which can be O, N, or S, or when taken together X₁ and X₂ form a cyclic boron amide-ester where said chain contains from 2-20 carbon atoms and, optionally, 1-3 heteroatoms which can be O, N, or S, or Xᵢ and R₁ together form a cyclic ring where said ring contains 2 to 10 carbon atoms and, optionally, 1-3 heteroatoms which can be O, N, or S, and X₂ is hydroxyl, halogen, NR₄R₅, C1-C6 alkoxy, or X₁ and R₃ together form a cyclic ring where said ring contains 3 to 10 carbon atoms and, optionally, 1-3 heteroatoms which can be O, N, or S, and X₂ is hydroxyl, halogen, NR₄R₅, or C1-C6 alkoxy;
   Y₁ and Y₂ are independently hydrogen, alkyl, cycloalkyl, alkoxy, alkenyl, alkynyl, amino, aminosulfonyl, aminocarbonyl, carbonyl, alkylaryl, aryl, aryloxy, carboxyl, cyano, halogen, heteroaryl, heteroaryloxy, heterocyclyl, sulfido, sulfonyl, or sulfoxido, or taken together Y₁ and Y₂ form a cyclic structure containing from 3-12 carbon atoms and, optionally, 1-3 heteroatoms which can be O, N, or S;
   or a salt thereof;
   provided that, when R₁ is —C(O)R₄, R₂ is hydrogen, R₃ is a phenyl group having two substituents consisting of a hydroxyl at the 2-position and a carboxylic acid at the 3-position relative to the group containing Y₁ and Y₂, X₁ and X₂ are hydroxyl or X₁ is hydroxyl and X₂ is replaced by the ortho-hydroxyl oxygen of R₃ such that a 6-membered ring is formed, and Y₁ and Y₂ are hydrogen, R₄ is not unsubstituted C1 alkyl;
   in combination with a β-lactam antibiotic in the manufacture of a medicament for the treatment of a bacterial infection.

Another aspect is for a composition for use in combination with a β-lactam antibiotic in reducing a bacterial infection comprising:

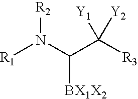

wherein R₁ is —C(O)R₄; —C(O)NR₄R₅; —C(O)OR₄; —S(O)₂R₄, —C(═NR₄R₅)R₄, —C(═NR₄R₅)NR₄R₅, hydrogen, or is selected from the group consisting of:
(a) aryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido,
(b) heteroaryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido, and
(c) heterocyclic group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido;
   R₂ is hydrogen, or is selected from the group consisting of:
(a) C1-C6 alkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the C1-C6 carbons comprise part of said oxyimino group, sulfido, and sulfoxido,
(b) C3-C7 cycloalkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the cycloalkyl group other than the one attached to the rest of the molecule comprise part of said oxyimino group, sulfido, and sulfoxido,
(c) aryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido,
(d) heteroaryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido, and
(e) heterocyclic group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, sulfido, and sulfoxido;
   R₃ is an aryl or heteroaryl group substituted with from 1 to 4 substituents wherein one of the substituents is a hydroxyl or amino group located at the 2 position relative to the group containing Y₁ and Y₂, and wherein the remaining substituents are selected from the group consisting of hydroxyl, alkyl, cycloalkyl, alkoxy, alkenyl, alkynyl, amino, aminocarbonyl, carbonyl, aminosulfonyl, alkylaryl, aryl, aryloxy, carboxyl, cyano, guanidino, halogen, heteroaryl, heterocyclyl, sulfido, sulfonyl, sulfoxido, sulfonic acid, sulfate, and thiol;
   R₄ is selected from the group consisting of:
(a) C1-C10 alkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the C1-C10 carbons comprise part of said oxyimino group, sulfido, and sulfoxido,
(b) C3-C10 cycloalkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the cycloalkyl group other than the one attached to the rest of the molecule comprise part of said oxyimino group, sulfido, and sulfoxido,
(c) aryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido,
(d) heteroaryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido, and
(e) heterocyclic group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, sulfido, and sulfoxido;
   R₅ is hydrogen or is selected from the group consisting of:
(a) C1-C6 alkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the C1-C10 carbons comprise part of said oxyimino group, sulfido, and sulfoxido,
(b) C3-C7 cycloalkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the cycloalkyl group other than the one attached to the rest of the molecule comprise part of said oxyimino group, sulfido, and sulfoxido,
(c) aryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido,
(d) heteroaryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido, and
(e) heterocyclic group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, sulfido, and sulfoxido;
   X₁ and X₂ are independently hydroxyl, halogen, NR₄R₅, C1-C6 alkoxy, or when taken together X₁ and X₂ form a cyclic boron ester where said chain or ring contains from 2 to 20 carbon atoms and, optionally, 1-3 heteroatoms which can be O, N, or S, or when taken together X₁ and X₂ form a cyclic boron amide where said chain or ring contains from 2 to 20 carbon atoms and, optionally, 1-3 heteroatoms which can be O, N, or S, or when taken together X₁ and X₂ form a cyclic boron amide-ester where said chain contains from 2-20 carbon atoms and, optionally, 1-3 heteroatoms which can be O, N, or S, or X₁ and R₁ together form a cyclic ring where said ring contains 2 to 10 carbon atoms and, optionally, 1-3 heteroatoms which can be O, N, or S, and X₂ is hydroxyl, halogen, NR₄R₅, C1-C6 alkoxy, or X₁ and R₃ together form a cyclic ring where said ring contains 3 to 10 carbon atoms and, optionally, 1-3 heteroatoms which can be O, N, or S, and X₂ is hydroxyl, halogen, NR₄R₅, or C1-C6 alkoxy;
   Y₁ and Y₂ are independently hydrogen, alkyl, cycloalkyl, alkoxy, alkenyl, alkynyl, amino, aminosulfonyl, aminocarbonyl, carbonyl, alkylaryl, aryl, aryloxy, carboxyl, cyano, halogen, heteroaryl, heteroaryloxy, heterocyclyl, sulfido, sulfonyl, or sulfoxido, or taken together Y₁ and Y₂ form a cyclic structure containing from 3-12 carbon atoms and, optionally, 1-3 heteroatoms which can be O, N, or S;
   or a salt thereof;
   provided that, when R₁ is —C(O)R₄, R₂ is hydrogen, R₃ is a phenyl group having two substituents consisting of a hydroxyl at the 2-position and a carboxylic acid at the 3-position relative to the group containing Y₁ and Y₂, X₁ and X₂ are hydroxyl or X₁ is hydroxyl and X₂ is replaced by the ortho-hydroxyl oxygen of R₃ such that a 6-membered ring is formed, and Y₁ and Y₂ are hydrogen, R₄ is not unsubstituted C1 alkyl.

Other objects and advantages will become apparent to those skilled in the art upon reference to the detailed description that hereinafter follows.

### BRIEF DESCRIPTION OF THE FIGURES

FIG. 1. General synthetic scheme (scheme 1) for the synthesis of α-amidoboronic acids using an isopropyl ester derived from 3-boronobenzoic acid compounds.

FIG. 2. General synthetic scheme (scheme 2) for the synthesis of α-amidoboronic acids using a tert-butyl ester derived from 3-boronobenzoic acid compounds.

FIG. 3. Equilibrium between the boronic acid open chain form and the boronic ester cyclic form of compounds possessing an ortho-phenol group.

FIG. 4. Structure of three beta-lactam antibiotics, PZ-601, ME1036, and BAL30072.

### DETAILED DESCRIPTION OF THE INVENTION

Applicants specifically incorporate the entire contents of all cited references in this disclosure. Further, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

The present invention relates generally to novel α-aminoboronic acids and their derivatives which act as broad-spectrum inhibitors of beta-lactamase enzymes. Beta-lactamases hydrolyze beta-lactam antibiotics, and are therefore an important cause of β-lactam antibiotic resistance. The compounds of the recent invention, particularly when administered in combination with a β-lactam antibiotic, overcome this resistance mechanism and render beta-lactamase producing bacteria susceptible to the β-lactam antibiotic. The present invention also relates to pharmaceutical compositions comprising a compound of the present invention, or salt thereof, an optional beta-lactam antibiotic, and a pharmaceutically acceptable excipient. The present invention also relates to a method for treating a bacterial infection in a mammal by administration of a therapeutically acceptable amount of the aforementioned pharmaceutical compositions. The present invention also relates to a method for increasing the effectiveness of a beta-lactam antibiotic in mammals by administering an effective amount of a compound of the present invention in combination with an effective amount of such beta-lactam antibiotic.

### DEFINITIONS

In the context of this disclosure, a number of terms shall be utilized.

As used herein, the term “about” or “approximately” means within 20%, preferably within 10%, and more preferably within 5% of a given value or range.

The term “antibiotic” is used herein to describe a compound or composition which decreases the viability of a microorganism, or which inhibits the growth or reproduction of a microorganism. “Inhibits the growth or reproduction” means increasing the generation cycle time by at least 2-fold, preferably at least 10-fold, more preferably at least 100-fold, and most preferably indefinitely, as in total cell death. As used in this disclosure, an antibiotic is further intended to include an antimicrobial, bacteriostatic, or bactericidal agent. Non-limiting examples of antibiotics useful according to this aspect of the invention include penicillins, cephalosporins, aminoglycosides, sulfonamides, macrolides, tetracyclins, lincosides, quinolones, chloramphenicol, vancomycin, metronidazole, rifampin, isoniazid, spectinomycin, trimethoprim, sulfamethoxazole, and others.

The term “beta-lactam antibiotic” is used to designate compounds with antibiotic properties containing a beta-lactam functionality. Non-limiting examples of beta-lactam antibiotics useful according to this aspect of the invention include penicillins, cephalosporins, penems, carbapenems, and monobactams. Beta-lactam antibiotics are effective (in the absence of resistance) against a wide range of bacterial infections. These include those caused by both gram-positive and gram-negative bacteria, for example, bacteria of the genus Staphylococcus (such as Staphylococcus aureus and Staphylococcus epidermidis), Streptococcus (such as Streptococcus agalactine, Streptococcus pneumoniae and Streptococcus faecalis), Micrococcus (such as Micrococcus luteus), Bacillus (such as Bacillus subtilis), Listerella (such as Listerella monocytogenes), Escherichia (such as Escherichia coli), Klebsiella (such as Klebsiella pneumoniae), Proteus (such as Proteus mirabilis and Proteus vulgaris), Salmonella (such as Salmonella typhosa), Shigella (such as Shigella sonnei), Enterobacter (such as Enterobacter aerogenes and Enterobacter cloacae), Serratia (such as Serratia marcescens), Pseudomonas (such as Pseudomonas aeruginosa), Acinetobacter (such as Acinetobacter anitratus), Nocardia (such as Nocardia autotrophica), and Mycobacterium (such as Mycobacterium fortuitum).

The term “beta-lactamase” means an enzyme produced by a bacteria that has the ability to hydrolyze the beta-lactam ring of beta-lactam antibiotics. Such enzymes are often classified into 4 major classes (Classes A, B, C, and D) according to the so-called Ambler classification scheme, based principally on protein homology.

The term “beta-lactamase inhibitors with broad-spectrum functionality” as used herein refers to the ability of an inhibitor to inhibit a broad range of beta-lactamase enzymes, spanning multiple subtypes from multiple classes (for example numerous enzyme subtypes from both Ambler Class A and Ambler Class C). In some embodiments, beta-lactamase enzyme(s) from at least two classes of beta-lactamase enzymes are inhibited by a compound disclosed herein, with preferred embodiments being those where beta-lactamase enzyme(s) from more than two classes of beta-lactamase enzymes are inhibited by a compound disclosed herein.

The term “comprising” is intended to include embodiments encompassed by the terms “consisting essentially of” and “consisting of”. Similarly, the term “consisting essentially of” is intended to include embodiments encompassed by the term “consisting of”.

The terms “effective amount”, “therapeutically effective amount”, and “therapeutically effective period of time” are used to denote known treatments at dosages and for periods of time effective to show a meaningful patient benefit, i.e., healing of conditions associated with bacterial infection, and/or bacterial drug resistance. Preferably, such administration should be parenteral, oral, sublingual, transdermal, topical, intranasal, intratracheal, or intrarectal. When administered systemically, the therapeutic composition is preferably administered at a sufficient dosage to attain a blood level of inhibitor of at least about 100 μg/mL, more preferably about 1 mg/mL, and still more preferably about 10 mg/mL. For localized administration, much lower concentrations than this may be effective, and much higher concentrations may be tolerated.

The term “mammal” refers to a human, a non-human primate, canine, feline, bovine, ovine, porcine, murine, or other veterinary or laboratory mammal. Those skilled in the art recognize that a therapy which reduces the severity of a pathology in one species of mammal is predictive of the effect of the therapy on another species of mammal.

### Chemical Definitions

The term alkyl means both straight and branched chain alkyl moieties of 1-12 carbons, preferably of 1-8 carbon atoms.

The term alkenyl means both straight and branched alkenyl moieties of 2-8 carbon atoms containing at least one double bond, and no triple bond, preferably the alkenyl moiety has one or two double bonds. Such alkenyl moieties may exist in the E or Z conformations; the compounds of this invention include both conformations.

The term alkynyl includes both straight chain and branched alkynyl moieties containing 2-6 carbon atoms containing at least one triple bond, preferably the alkynyl moiety has one or two triple bonds.

The term cycloalkyl refers to an alicyclic hydrocarbon group having 3-7 carbon atoms.

The term halogen is defined as Cl, Br, F, and I.

Aryl is defined as an aromatic hydrocarbon moiety selected from the group: phenyl, α-naphthyl, β-naphthyl, biphenyl, anthryl, tetrahydronaphthyl, fluorenyl, indanyl, biphenylenyl, acenaphthenyl, groups.

Heteroaryl is defined as an aromatic heterocyclic ring system (monocyclic or bicyclic) where the heteroaryl moieties are selected from, but not limited to: (1) furan, thiophene, indole, azaindole, oxazole, thiazole, isoxazole, isothiazole, imidazole, N-methylimidazole, pyridine, pyrimidine, pyrazine, pyrrole, N-methylpyrrole, pyrazole, N-methylpyrazole, 1,3,4-oxadiazole, 1,2,4-triazole, 1-methyl-1,2,4-triazole, 1H-tetrazole, 1-methyltetrazole, 1,2,4-thiadiazole, 1,3,4-thiadiazole, 1,2,3-thiadiazole, 1,2,3-triazole, 1-methyl-1,2,3-triazole, benzoxazole, benzothiazole, benzofuran, benzisoxazole, benzimidazole, N-methylbenzimidazole, azabenzimidazole, indazole, quinazoline, quinoline, and isoquinoline; (2) a bicyclic aromatic heterocycle where a phenyl, pyridine, pyrimidine or pyridizine ring is: (a) fused to a 6-membered aromatic (unsaturated) heterocyclic ring having one nitrogen atom; (b) fused to a 5 or 6-membered aromatic (unsaturated) heterocyclic ring having two nitrogen atoms; (c) fused to a 5-membered aromatic (unsaturated) heterocyclic ring having one nitrogen atom together with either one oxygen or one sulfur atom; or (d) fused to a 5-membered aromatic (unsaturated) heterocyclic ring having one heteroatom selected from O, N or S.

Arylalkyl is defined as aryl-C1-C6alkyl-. Arylalkyl moieties include benzyl, 1-phenylethyl, 2-phenylethyl, 3-phenylpropyl, 2-phenylpropyl and the like.

Alkylaryl is defined as C1-C6alkyl-aryl-

Heteroarylalkyl is defined as heteroaryl-C1-C6alkyl-.

Alkylheteroaryl is defined as C1-C6alkyl-heteroaryl-.

Heterocyclyl is defined as a saturated or partially saturated heterocyclic moiety selected from, but not limited to; aziridinyl, azetidinyl, 1,4-dioxanyl, hexahydroazepinyl, piperazinyl, piperidinyl, pyrrolidinyl, morpholinyl, thiomorpholinyl, dihydrobenzimidazolyl, dihydrobenzofuranyl, dihydrobenzothienyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, dihydro-1,4-dioxanyl, tetrahydrofuranyl, tetrahydrothienyl, tetrahydroquinolinyl, and tetrahydroisoquinolinyl.

Alkoxy is defined as C1-C6alkyl-O—.

Cycloalkoxy is defined as C3-C7cycloalkyl-O—.

Aryloxy is defined as aryl-O—.

Heteroaryloxy is defined as heteroaryl-O—.

Heterocyclyloxy is defined as C3-C7heterocyclyl-O—.

Sulfonic acid is defined as —SO₃H.

Sulfate is defined as —OSO₃H.

Amino is defined as —NH₂.

Cyano is defined as —CN

Hydroxyl is defined as —OH

Thiol is defined as —SH

Carboxyl is defined as —CO₂H.

Trialkylammonium is defined as (A1)(A2)(A3)N+— where A1, A2 and A3 are independently alkyl, cycloalkyl, heterocyclyl and the nitrogen is positively charged.

Carbonyl is defined as —C(O)— where the carbon is optionally substituted and also attached to the rest of the molecule.

Aminocarbonyl is defined as —C(O)—N—, where the carbon is optionally substituted and the nitrogen is attached to the rest of the molecule.

Oxycarbonyl is defined as —C(O)—O—, where the carbon is optionally substituted and the oxygen is attached to the rest of the molecule.

Aminosulfonyl is defined as —S(O)₂—N— where the sulfur is optionally substituted and the nitrogen is attached to the rest of the molecule.

Sulfonyl is defined as —S(O)₂— where the sulfur is bonded to an optional substituent and also to the rest of the molecule.

Guanidino is defined as —N1(H)—C(NH)—N2(H)— where N1 is optionally substituted and N2 is attached to the rest of the molecule.

Oxyimino is defined as (═N—O-A) where the nitrogen is double bonded to a carbon which is attached to the rest of the molecule and A can be hydrogen, optionally substituted: alkyl, cycloalkyl, aryl, heteroaryl, heterocyclyl.

Sulfido is defined as —S— where sulfur is bound to an optional substituent and also to the rest of the molecule.

Sulfoxido is defined as —S(O)— where sulfur is bound to an optional substituent and also to the rest of the molecule.

Where a group or atom is described as “optionally substituted” one or more of the following substituents may be present on that group or atom: hydroxyl, halogen, carboxyl, cyano, thiol, amino, sulfonic acid, sulfate, alkyl, cycloalkyl, alkoxy, alkenyl, alkynyl, aryl, heteroaryl, heterocyclyl, cycloalkoxy, heterocyclyloxy, aryloxy, heteroaryloxy, arylakyl, alkylaryl, heteroarylalkyl, alkylheteroaryl, trialkylammonium. Optional substituents may be attached to the group or atom which they substitute in a variety of ways, either directly or through a connecting group of which the following are examples: alkyl, amine, amide, ester, ether, thioether, sulfonamide, sulfamide, sulfoxide, urea. As appropriate an optional substituent may itself be further substituted by another substituent, the latter being connected directly to the former or through a connecting group such as those exemplified above.

### Beta-Lactamase Inhibitors

The present disclosure relates to compounds of formula I:

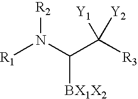

wherein R₁ is —C(O)R₄; —C(O)NR₄R₅; —C(O)OR₄; —S(O)₂R₄, —C(═NR₄R₅)R₄, —C(═NR₄R₅)NR₄R₅, hydrogen, or is selected from the group consisting of:
(a) aryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido,
(b) heteroaryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido, and
(c) heterocyclic group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido;
   R₂ is hydrogen, or is selected from the group consisting of:
(a) C1-C6 alkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino (wherein any of the C1-C6 carbons comprise part of said oxyimino group), sulfido, and sulfoxido,
(b) C3-C7 cycloalkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino (wherein any of the carbons of the cycloalkyl group other than the one attached to the rest of the molecule comprise part of said oxyimino group), sulfido, and sulfoxido,
(c) aryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido,
(d) heteroaryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido, and
(e) heterocyclic group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino (wherein any of the carbons of the heterocyclic group other than the one attached to the rest of the molecule comprise part of said oxyimino group), sulfido, and sulfoxido;
   R₃ is an aryl or heteroaryl group substituted with from 1 to 4 substituents wherein one of the substituents is a hydroxyl or amino group located at the 2 position relative to the group containing Y₁ and Y₂, and wherein the remaining substituents are selected from the group consisting of hydroxyl, alkyl, cycloalkyl, alkoxy, alkenyl, alkynyl, amino, aminocarbonyl, carbonyl, aminosulfonyl, alkylaryl, aryl, aryloxy, carboxyl, cyano, guanidino, halogen, heteroaryl, heterocyclyl, sulfido, sulfonyl, sulfoxido, sulfonic acid, sulfate, and thiol;
   R₄ is selected from the group consisting of:
(a) C1-C10 alkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino (wherein any of the C1-C10 carbons comprise part of said oxyimino group), sulfido, and sulfoxido,
(b) C3-C10 cycloalkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino (wherein any of the carbons of the cycloalkyl group other than the one attached to the rest of the molecule comprise part of said oxyimino group), sulfido, and sulfoxido,
(c) aryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido,
(d) heteroaryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido, and
(e) heterocyclic group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino (wherein any of the carbons of the heterocyclic group other than the one attached to the rest of the molecule comprise part of said oxyimino group), sulfido, and sulfoxido;
   R₅ is hydrogen or is selected from the group consisting of:
(a) C1-C6 alkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino (wherein any of the C1-C10 carbons comprise part of said oxyimino group), sulfido, and sulfoxido,
(b) C3-C7 cycloalkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino (wherein any of the carbons of the cycloalkyl group other than the one attached to the rest of the molecule comprise part of said oxyimino group), sulfido, and sulfoxido,
(c) aryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido,
(d) heteroaryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido, and
(e) heterocyclic group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino (wherein any of the carbons of the heterocyclic group other than the one attached to the rest of the molecule comprise part of said oxyimino group), sulfido, and sulfoxido;
   X₁ and X₂ are independently hydroxyl, halogen, NR₄R₅, C1-C6 alkoxy, or when taken together X₁ and X₂ form a cyclic boron ester where said chain or ring contains from 2 to 20 carbon atoms and, optionally, 1-3 heteroatoms which can be O, N, or S, or when taken together X₁ and X₂ form a cyclic boron amide where said chain or ring contains from 2 to 20 carbon atoms and, optionally, 1-3 heteroatoms which can be O, N, or S, or when taken together X₁ and X₂ form a cyclic boron amide-ester where said chain contains from 2-20 carbon atoms and, optionally, 1-3 heteroatoms which can be O, N, or S, or X₁ and R₁ together form a cyclic ring where said ring contains 2 to 10 carbon atoms and, optionally, 1-3 heteroatoms which can be O, N, or S, and X₂ is hydroxyl, halogen, NR₄R₅, C1-C6 alkoxy, or X₁ and R₃ together form a cyclic ring where said ring contains 3 to 10 carbon atoms and, optionally, 1-3 heteroatoms which can be O, N, or S, and X₂ is hydroxyl, halogen, NR₄R₅, or C1-C6 alkoxy;
   Y₁ and Y₂ are independently hydrogen, alkyl, cycloalkyl, alkoxy, alkenyl, alkynyl, amino, aminosulfonyl, aminocarbonyl, carbonyl, alkylaryl, aryl, aryloxy, carboxyl, cyano, halogen, heteroaryl, heteroaryloxy, heterocyclyl, sulfido, sulfonyl, or sulfoxido, or taken together Y₁ and Y₂ form a cyclic structure containing from 3-12 carbon atoms and, optionally, 1-3 heteroatoms which can be O, N, or S.

Preferred embodiments are those compounds of Formula (I) wherein R₁ is —C(O)R₄; R₂ is hydrogen; R₃ is an aryl or heteroaryl group substituted with from 2 to 4 substituents wherein one of the substituents is a hydroxyl or amino group located at the 2-position relative to the group containing Y₁ and Y₂, and a second substituent is a carboxylic acid group and wherein the remaining substituents are selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido;

R₄ is selected from the group consisting of:
(a) C1-C10 alkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino (wherein any of the C1-C10 carbons comprise part of said oxyimino group), sulfido, and sulfoxido,
(b) C3-C10 cycloalkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino (wherein any of the carbons of the cycloalkyl group other than the one attached to the rest of the molecule comprise part of said oxyimino group), sulfido, and sulfoxido,
(c) aryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido,
(d) heteroaryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido, and
(e) heterocyclic group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino (wherein any of the carbons of the heterocyclic group other than the one attached to the rest of the molecule comprise part of said oxyimino group), sulfido, and sulfoxido;
   X₁ and X₂ are hydroxyl, or when taken together X₁ and X₂ form a cyclic boron ester where said chain or ring contains from 2 to 20 carbon atoms and, optionally, 1-3 heteroatoms which can be O, N, or S, or X₁ and R₁ together form a cyclic ring where said ring contains 2 to 10 carbon atoms and, optionally, 1-3 heteroatoms which can be O, N, or S, and X₂ is hydroxyl, or X₁ and R₃ together form a cyclic ring where said ring contains 3 to 10 carbon atoms and, optionally, 1-3 heteroatoms which can be O, N, or S and X₂ is hydroxyl;
   Y₁ and Y₂ are independently hydrogen, alkyl, cycloalkyl, alkoxy, alkenyl, alkynyl, amino, aminosulfonyl, aminocarbonyl, carbonyl, alkylaryl, aryl, aryloxy, carboxyl, cyano, halogen, heteroaryl, heteroaryloxy, heterocyclyl, sulfido, sulfonyl, or sulfoxido.

Other preferred embodiments are those compounds of Formula (I) wherein R₁ is —C(O)R₄; R₂ is hydrogen; R₃ is an aryl group having a hydroxyl at the 2-position and a carboxylic acid at the 3-position relative to the group containing Y₁ and Y₂; R₄ is C1-C10 alkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino (wherein any of the C1-C10 carbons comprise part of said oxyimino group), sulfido, and sulfoxido; X₁ and X₂ are hydroxyl, or X₁ is hydroxyl and X₂ is replaced by the ortho-hydroxyl oxygen of R₃ such that a 6-membered ring is formed; and Y₁ and Y₂ are hydrogen.

Other preferred embodiments are those compounds of Formula (I) wherein R₁ is —C(O)R₄; R₂ is hydrogen; R₃ is an aryl group having a hydroxyl at the 2-position and a carboxylic acid at the 3-position relative to the group containing Y₁ and Y₂; R₄ is C3-C10 cycloalkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino (wherein any of the carbons of the cycloalkyl group other than the one attached to the rest of the molecule comprise part of said oxyimino group), sulfido, and sulfoxido; X₁ and X₂ are hydroxyl, or X₁ is hydroxyl and X₂ is replaced by the ortho-hydroxyl oxygen of R₃ such that a 6-membered ring is formed; and Y₁ and Y₂ are hydrogen.

Other preferred embodiments are those compounds of Formula (I) wherein R₁ is —C(O)R₄; R₂ is hydrogen; R₃ is an aryl group having a hydroxyl at the 2-position and a carboxylic acid at the 3-position relative to the group containing Y₁ and Y₂; R₄ is aryl or heteroaryl substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido; X₁ and X₂ are hydroxyl, or X₁ is hydroxyl and X₂ is replaced by the ortho-hydroxyl oxygen of R₃ such that a 6-membered ring is formed; and Y₁ and Y₂ are hydrogen.

Other preferred embodiments are those compounds of Formula (I) wherein R₁ is —C(O)R₄; R₂ is hydrogen; R₃ is an aryl group having a hydroxyl at the 2-position and a carboxylic acid at the 3-position relative to the group containing Y₁ and Y₂; R₄ is a heterocycle substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino (wherein any of the carbons of the heterocyclic group other than the one attached to the rest of the molecule comprise part of said oxyimino group), sulfido, and sulfoxido; X₁ and X₂ are hydroxyl, or X₁ is hydroxyl and X₂ is replaced by the ortho-hydroxyl oxygen of R₃ such that a 6-membered ring is formed; and Y₁ and Y₂ are hydrogen.

### Beta-Lactamase Inhibitor Synthesis

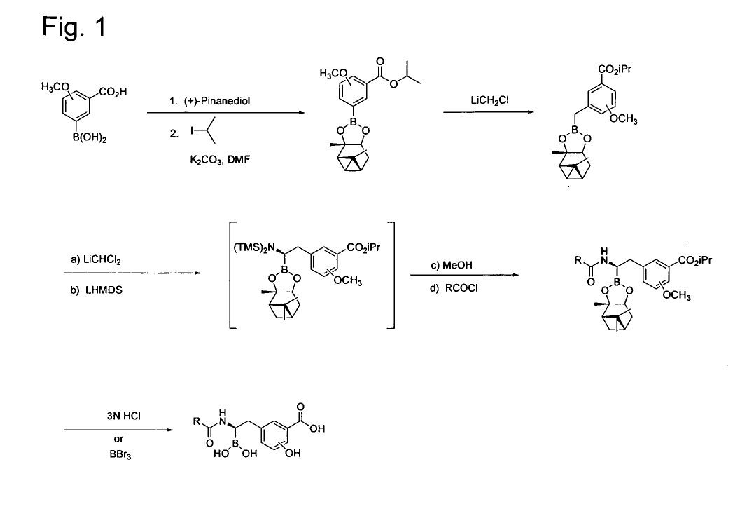

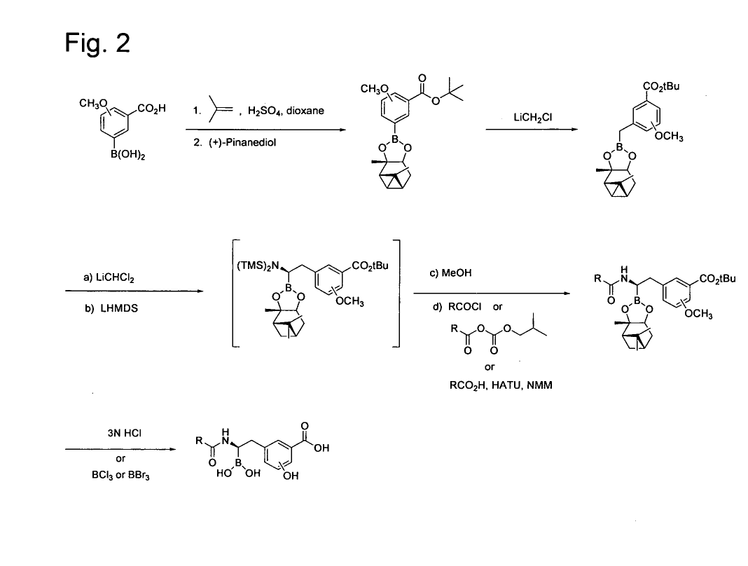

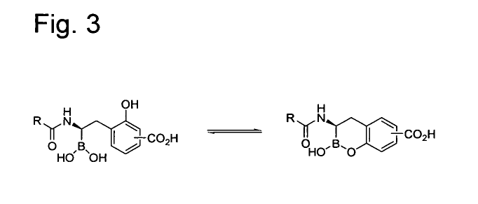

The compounds of the current invention can be synthesized using the general routes depicted in FIGS. 1 and 2. In FIG. 1, the boronic acid is first converted to the chiral boronic ester by reaction with (+)-pinanediol, and the carboxylic acid group is subsequently protected as the isopropyl ester using 2-iodopropane and potassium carbonate in N,N-dimethylformamide (DMF). In FIG. 2, the carboxylic acid group is first protected as the tert-butyl ester using 2-methylpropene in the presence of catalytic sulfuric acid, and the boronic acid is then subsequently converted to the chiral boronic ester with (+)-pinanediol. In both routes, homologation using (chloromethyl)lithium as described by Sadhu and Matteson, Organometallics, 1985, 4, 1687-1689 affords the benzylboronic esters. Conversion to the bis(trimethylsilyl)amine intermediates can be achieved using the conditions described by Schoichet et al., J. Am. Chem. Soc. 2003, 125, 685-695. These intermediates could then be converted to the desired amide by reaction with an acid chloride or other active ester such as that derived from the reaction of a carboxylic acid with isobutyl chloroformate or from the reaction of a carboxylic acid with a tetramethyluronium agent such as O-(7-Azabenzotriazol-1-yl)-N,N,N′,N′-tetramethyluronium hexafluorophosphate (HATU). Removal of the pinanediol groups and deprotection of the carboxylic acids and phenol groups can be accomplished in one step under acidic conditions such as aqueous HCl in dioxane or BCl₃ or BBr₃ in dichloromethane. Based on literature precedent, it is assumed that Applicants obtain predominantly the 1-(R) enantiomer, although one skilled in the art will recognize that minor amounts of the 1-(S) isomer may be present in the reaction products. Also, there is a possibility that those compounds possessing an ortho-hydroxy group on the aryl ring of R₃ can exist either as the free boronic acid or as the cyclic boronate ester, or as a mixture of the cyclic form and the open chain form as depicted in FIG. 3 (Strynadka et al. Biochemistry, 2000, 39(18), 5312-5321).

### Administration of Beta-Lactamase Inhibitors

Beta-lactamase inhibitors can be administered to subjects in a biologically compatible form suitable for pharmaceutical administration in vivo to, e.g., increase antibacterial activity of beta-lactam antibiotics. Administration of a beta-lactamase inhibitor as described herein can be in any pharmacological form including a therapeutically active amount of a beta-lactamase inhibitor alone or in combination with a pharmaceutically acceptable carrier.

A therapeutically active amount of a beta-lactamase inhibitor may vary according to factors such as the disease state, age, sex, and weight of the subject, and the ability of the beta-lactamase inhibitor to elicit a desired response in the subject. Dosage regimes may be adjusted to provide the optimum therapeutic response. For example, several divided doses may be administered daily, or the dose may be proportionally reduced as indicated by the exigencies of the therapeutic situation.

The therapeutic or pharmaceutical compositions can be administered by any suitable route known in the art including, for example, intravenous, subcutaneous, intramuscular, transdermal, intrathecal, or intracerebral or administration to cells in ex vivo treatment protocols. Administration can be either rapid as by injection or over a period of time as by slow infusion or administration of slow release formulation.

A beta-lactamase inhibitor can also be linked or conjugated with agents that provide desirable pharmaceutical or pharmacodynamic properties. For example, a beta-lactamase inhibitor can be coupled to any substance known in the art to promote penetration or transport across the blood-brain barrier such as an antibody to the transferrin receptor, and administered by intravenous injection (see, e.g., Friden P M et al., Science 259:373-77 (1993)). Furthermore, a beta-lactamase inhibitor can be stably linked to a polymer such as polyethylene glycol to obtain desirable properties of solubility, stability, half-life, and other pharmaceutically advantageous properties (see, e.g., Davis et al., Enzyme Eng. 4:169-73 (1978); Burnham N L, Am. J. Hosp. Pharm. 51:210-18 (1994)).

Furthermore, a beta-lactamase inhibitor can be in a composition which aids in delivery into the cytosol of a cell. For example, the beta-lactamase inhibitor may be conjugated with a carrier moiety such as a liposome that is capable of delivering the beta-lactamase inhibitor into the cytosol of a cell. Such methods are well known in the art (see, e.g., Amselem S et al., Chem. Phys. Lipids 64:219-37 (1993)). Alternatively, a beta-lactamase inhibitor can be modified to include specific transit peptides or fused to such transit peptides which are capable of delivering their beta-lactamase inhibitor into a cell. In addition, the beta-lactamase inhibitor can be delivered directly into a cell by microinjection.

The compositions are usually employed in the form of pharmaceutical preparations. Such preparations are made in a manner well known in the pharmaceutical art. One preferred preparation utilizes a vehicle of physiological saline solution, but it is contemplated that other pharmaceutically acceptable carriers such as physiological concentrations of other non-toxic salts, five percent aqueous glucose solution, sterile water, or the like may also be used. As used herein, “pharmaceutically acceptable carrier” includes any and all solvents, dispersion media, coatings, antibacterial and antifungal agents, isotonic and absorption delaying agents, and the like. The use of such media and agents for pharmaceutically active substances is well known in the art. Except insofar as any standard media or agent is incompatible with the active compound, use thereof in the therapeutic compositions is contemplated. Supplementary active compounds can also be incorporated into the compositions. It may also be desirable that a suitable buffer be present in the composition. Such solutions can, if desired, be lyophilized and stored in a sterile ampoule ready for reconstitution by the addition of sterile water for ready injection. The primary solvent can be aqueous or alternatively non-aqueous. A beta-lactamase inhibitor can also be incorporated into a solid or semi-solid biologically compatible matrix which can be implanted into tissues.

The carrier can contain other pharmaceutically-acceptable excipients for modifying or maintaining the pH, osmolarity, viscosity, clarity, color, sterility, stability, rate of dissolution, or odor of the formulation. Such excipients are those substances usually and customarily employed to formulate dosages for parenteral administration in either unit dosage or multi-dose form or for direct infusion by continuous or periodic infusion.

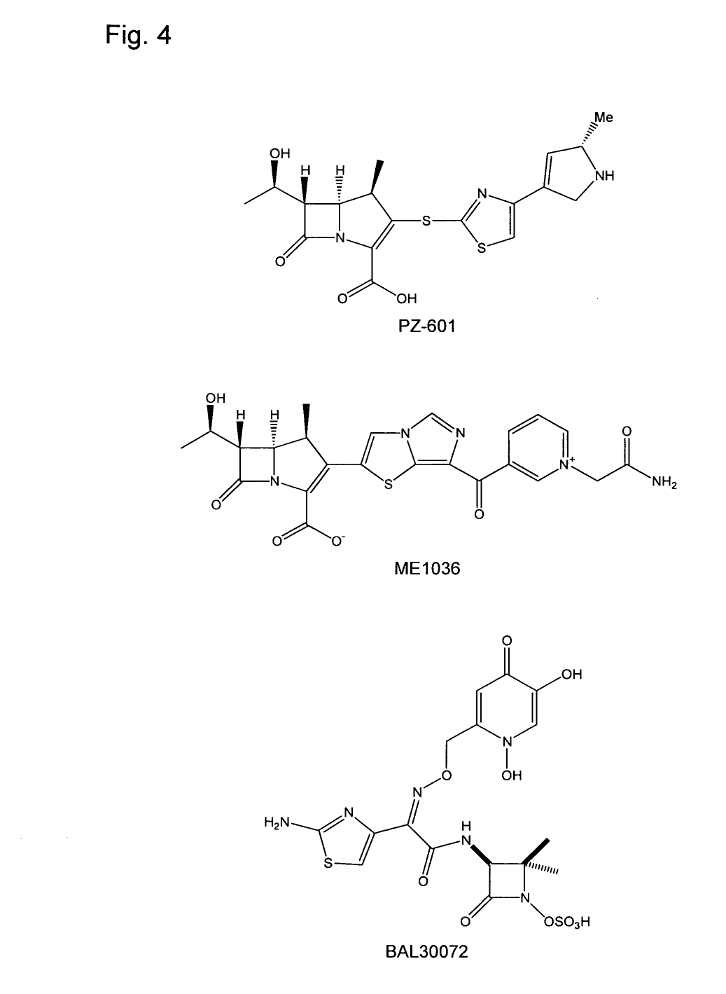

In some embodiments, the pharmaceutical compositions further comprise an effective amount of a beta-lactam antibiotic. Exemplary β-lactam antibiotics include penicillins, cephalosporins, carbapenems, monobactams, bridged monobactams, or a combination thereof. Pencillins include, but are not limited to, benzathine penicillin, benzylpenicillin, phenoxymethylpenicillin, procaine penicillin, oxacillin, methicillin, dicloxacillin, flucloxacillin, temocillin, amoxicillin, ampicillin, co-amoxiclav, azlocillin, carbenicillin, ticarcillin, mezlocillin, piperacillin, apalcillin, hetacillin, bacampicillin, sulbenicillin, mecicilam, pevmecillinam, ciclacillin, talapicillin, aspoxicillin, cloxacillin, nafcillin, pivampicillin, or a combination thereof. Cephalosporins include, but are not limited to, cephalothin, cephaloridin, cefaclor, cefadroxil, cefamandole, cefazolin, cephalexin, cephradine, ceftizoxime, cefoxitin, cephacetril, cefotiam, cefotaxime, cefsulodin, cefoperazone, ceftizoxime, cefinenoxime, cefinetazole, cephaloglycin, cefonicid, cefodizime, cefpirome, ceftazidime, ceftriaxone, cefpiramide, cefbuperazone, cefozopran, cefepim, cefoselis, cefluprenam, cefuzonam, cefpimizole, cefclidin, cefixime, ceftibuten, cefdinir, cefpodoxime axetil, cefpodoxime proxetil, cefteram pivoxil, cefetamet pivoxil, cefcapene pivoxil, cefditoren pivoxil, cefuroxime, cefuroxime axetil, loracarbacef, latamoxef, anti-methicillin-resistant Staphylococcus aureus (MRSA) cephalosporins (e.g., ceftobiprole or ceftaroline), or a combination thereof. Carbapenems include, but are not limited to, imipenem, meropenem, ertapenem, faropenem, doripenem, biapenem, panipenem, anti-MRSA carbapenems (e.g., PZ-601 or ME1036, see Expert Rev. Anti-Infect. Ther. (2008) 6:39-49), or a combination thereof. Monobactams include, but are not limited to, aztreonam, carumonam, BAL30072 (Basilea Poster F1-1173, Ann. Interscience Conf. Antimicrob. Agents Chemother. (2008)), or a combination thereof. See FIG. 4 for structures of PZ-601, ME1036, and BAL30072.

The beta-lactamase inhibitors or their pharmaceutically acceptable salts may be administered at the same time as the dose of beta-lactam antibiotics or separately. This may be carried out in the form of a mixture of the two active ingredients or in the form of a pharmaceutical combination of the two separate active ingredients.

The dosage of the beta-lactamase inhibitors and of their pharmaceutically acceptable salts may vary within wide limits and should naturally be adjusted, in each particular case, to the individual conditions and to the pathogenic agent to be controlled. In general, for a use in the treatment of bacterial infections, the daily dose may be between 0.250 g and 10 g per day, by the oral route in humans, or else between 0.25 g and 10 g per day by the intramuscular or intravenous route. Moreover, the ratio of the beta-lactamase inhibitor or of the pharmaceutically acceptable salt thereof to the beta-lactam antibiotic may also vary within wide limits and should be adjusted, in each particular case, to the individual conditions. In general, a ratio ranging from about 1:20 to about 1:1 is recommended.

Dose administration can be repeated depending upon the pharmacokinetic parameters of the dosage formulation and the route of administration used.

It is also provided that certain formulations containing a beta-lactamase inhibitor are to be administered orally. Such formulations are preferably encapsulated and formulated with suitable carriers in solid dosage forms. Some examples of suitable carriers, excipients, and diluents include lactose, dextrose, sucrose, sorbitol, mannitol, starches, gum acacia, calcium phosphate, alginates, calcium silicate, microcrystalline cellulose, polyvinylpyrrolidone, cellulose, gelatin, syrup, methyl cellulose, methyl- and propylhydroxybenzoates, talc, magnesium, stearate, water, mineral oil, and the like. The formulations can additionally include lubricating agents, wetting agents, emulsifying and suspending agents, preserving agents, sweetening agents, or flavoring agents. The compositions may be formulated so as to provide rapid, sustained, or delayed release of the active ingredients after administration to the patient by employing procedures well known in the art. The formulations can also contain substances that diminish proteolytic degradation and/or substances which promote absorption such as, for example, surface active agents.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms are dictated by and directly dependent on (a) the unique characteristics of the active compound and the particular therapeutic effect to be achieved and (b) the limitations inherent in the art of compounding such an active compound for the treatment of sensitivity in individuals. The specific dose can be readily calculated by one of ordinary skill in the art, e.g., according to the approximate body weight or body surface area of the patient or the volume of body space to be occupied. The dose will also be calculated dependent upon the particular route of administration selected. Further refinement of the calculations necessary to determine the appropriate dosage for treatment is routinely made by those of ordinary skill in the art. Such calculations can be made without undue experimentation by one skilled in the art in light of the activity disclosed herein in assay preparations of target cells. Exact dosages are determined in conjunction with standard dose-response studies. It will be understood that the amount of the composition actually administered will be determined by a practitioner, in the light of the relevant circumstances including the condition or conditions to be treated; the choice of composition to be administered; the age, weight, and response of the individual patient; the severity of the patient's symptoms; and the chosen route of administration.

Toxicity and therapeutic efficacy of such compounds can be determined by standard pharmaceutical procedures in cell cultures or experimental animals, for example, for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the methods disclosed herein, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

### Inhibition of Bacterial Growth

The present disclosure also provides methods for inhibiting bacterial growth, by e.g. reducing bacterial resistance to a β-lactam antibiotic, such methods comprising contacting a bacterial cell culture, or a bacterially infected cell culture, tissue, or organism, with a beta-lactamase inhibitor described herein. Preferably, the bacteria to be inhibited by administration of a beta-lactamase inhibitor of the invention are bacteria that are resistant to beta-lactam antibiotics. More preferably, the bacteria to be inhibited are beta-lactamase positive strains that are highly resistant to beta-lactam antibiotics. The terms “resistant” and “highly resistant” are well-understood by those of ordinary skill in the art (see, e.g., Payne et al., Antimicrobial Agents and Chemotherapy 38:767-772 (1994); Hanaki et al., Antimicrobial Agents and Chemotherapy 30:1120-1126 (1995)). Preferably, highly resistant bacterial strains are those against which the MIC of methicillin is >100 μg/mL. Preferably, slightly resistant bacterial strains are those against which the MIC of methicillin is >25 μg/mL.

These methods are useful for inhibiting bacterial growth in a variety of contexts. In certain preferred embodiments, the compound of the invention is administered to an experimental cell culture in vitro to prevent the growth of beta-lactam resistant bacteria. In certain other preferred embodiments the compound of the invention is administered to a mammal, including a human, to prevent the growth of beta-lactam resistant bacteria in vivo. The method according to this embodiment of the invention comprises administering a therapeutically effective amount of a beta-lactamase inhibitor for a therapeutically effective period of time to a mammal, including a human. Preferably, the beta-lactamase inhibitor is administered in the form of a pharmaceutical composition as described supra. In some embodiments, a beta-lactam antibiotic is co-administered with the beta-lactamase inhibitor as described supra.

Assays for the inhibition of beta-lactamase activity are well known in the art. For instance, the ability of a compound to inhibit beta-lactamase activity in a standard enzyme inhibition assay may be used (see, e.g., Page, Biochem J. 295:295-304 (1993)). Beta-lactamases for use in such assays may be purified from bacterial sources or, preferably, are produced by recombinant DNA techniques, since genes and cDNA clones coding for many beta-lactamases are known (see, e.g., Cartwright & Waley, Biochem J. 221:505-12 (1984)). Alternatively, the sensitivity of bacteria known, or engineered, to produce a beta-lactamase to an inhibitor may be determined. Other bacterial inhibition assays include agar disk diffusion and agar dilution (see, e.g., Traub & Leonhard, Chemotherapy 43:159-67 (1997)). Thus, a beta-lactamase can be inhibited by contacting the beta-lactamase enzyme with an effective amount of an inventive compound or by contacting bacteria that produce the beta-lactamase enzymes with an effective amount of such a compound so that the beta-lactamase in the bacteria is contacted with the inhibitor. The contacting may take place in vitro or in vivo. “Contacting” means that the beta-lactamase and the inhibitor are brought together so that the inhibitor can bind to the beta-lactamase. Amounts of a compound effective to inhibit a beta-lactamase may be determined empirically, and making such determinations is within the skill in the art. Inhibition includes both reduction and elimination of beta-lactamase activity.

### EXAMPLES

The disclosure herein is further defined in the following Examples. It should be understood that these Examples, while indicating preferred embodiments, are given by way of illustration only. From the above discussion and these Examples, one skilled in the art can ascertain the preferred features, and without departing from the spirit and scope thereof, can make various changes and modifications to adapt it to various uses and conditions.

### Example 1

### (1R)-1-(2-thiophene-2-yl-acetylamino)-2-(2-hydroxy-3-carboxyphenyl)ethyl-1-boronic acid

Step 1. Synthesis of 2-Methoxy-3-(2,9,9-Trimethyl-3,5-dioxa-4-bora-tricyclo[6.1.1.0^{2,6}]dec-4-yl)-benzoic acid. A solution of (+)-pinanediol (17.4 g, 102.0 mmole) and 3-borono-2-methoxybenzoic acid (20.0 g, 102.4 mmole) in tetrahydrofuran (THF, 140 mL) was stirred for 15 h at ambient temperature. The solution was concentrated in vacuo, and the residue was washed with hexanes to afford 29.6 g (88%) of the product as a slowly crystallizing white solid. ESI-MS m/z 331 (MH)⁺.

Step 2. Synthesis of 2-Methoxy-3-(2,9,9-Trimethyl-3,5-dioxa-4-bora-tricyclo[6.1.1.0^{2,6}]dec-4-yl)-benzoic acid isopropyl ester. A solution of 2-methoxy-3-(2,9,9-trimethyl-3,5-dioxa-4-bora-tricyclo[6.1.1.0^{2,6}]dec-4-yl)-benzoic acid (22.3 g, 67.6 mmole), 2-iodopropane (13.5 mL, 13.5 mmole) and potassium carbonate (18.7 g, 13.5 mmole) in N,N-dimethylformamide (DMF, 337 mL) was stirred at ambient temperature for 18 h. The reaction was quenched with water and extracted twice with ethyl acetate (EtOAc). The combined organic layers were washed with water, brine, dried (MgSO₄) and concentrated in vacuo. The residue was chromatographed on SiO₂ using a gradient of 40% dichloromethane (DCM)/hexane to 90% DCM/hexane to afford 20.3 g (81%) of product as a white solid. Electrospray ionization-mass spectrum (ESI-MS) m/z 373 (MH)⁺.

Step 3. Synthesis of 2-Methoxy-3-(2,9,9-Trimethyl-3,5-dioxa-4-bora-tricyclo[6.1.1.0^{2,6}]dec-4-ylmethyl)-benzoic acid isopropyl ester. To a solution of 2-methoxy-3-(2,9,9-trimethyl-3,5-dioxa-4-bora-tricyclo[6.1.1.0^{2,6}]dec-4-yl)-benzoic acid isopropyl ester (10.6 g, 28.49 mmole) and chloroiodomethane (2.6 mL, 35.61 mmole) in THF (84 mL) at −100° C. was added n-butyllithium (n-BuLi, 2.5 M in hexanes, 14.2 mL, 35.50 mmole) over 6 minutes. The solution was stirred at −100° C. for 45 min. The reaction was allowed to warm up gradually while stirring overnight. The reaction was quenched with water and extracted twice with ethyl acetate. The combined organic layers were washed with water, brine, dried (MgSO₄) and concentrated in vacuo. The residue was chromatographed on SiO₂ using a gradient of 40% DCM/hexane to 70% DCM/hexane to afford 15.1 g (71%) of product as a colorless oil. ESI-MS m/z 387 (MH)⁺.

Step 4. Synthesis of (1R)-2-Methoxy-3-[2-(2-Thiophen-2-yl-acetylamino)-2-(2,9,9-trimethyl-3,5-dioxa-4-bora-tricyclo[6.1.1.0^{2,6}]dec-4-yl)-ethyl]-benzoic acid isopropyl ester. To a solution of anhydrous dichloromethane (2.2 mL, 34.5 mmole) in THF (77 mL) at −100° C. was added n-BuLi (2.5M in hexanes, 10.4 mL, 25.8 mmole) over 15 min. The solution was stirred for 30 min at −100° C. at which point the microcrystalline LiCHCl₂ precipitate was visible. A solution of 2-methoxy-3-(2,9,9-trimethyl-3,5-dioxa-4-bora-tricyclo[6.1.1.0^{2,6}]dec-4-ylmethyl)-benzoic acid isopropyl ester (8.31 g, 21.5 mmole) in THF (16 mL) was added over 5 min by syringe. The mixture was stirred at −100° C. for 15 min, then warmed to 0° C. and held for 2 h. The solution was then cooled to −78° C. and a solution of lithium bis(trimethylsilyl)amide (LHMDS, 1.0 M in THF, 25.8 mL, 25.8 mmole) was added over 5 min. The reaction was allowed to warm gradually while stirring overnight. The mixture was then cooled to −10° C. and anhydrous methanol (1.04 mL, 25.8 mmole) was added. This stirred for 45 min, then the bath was removed and the solution stirred at ambient temperature for 1.25 h. After cooling to −78° C., 2-thiopheneacetyl chloride (3.45 mL, 27.9 mmole) was added and the solution stirred at −78° C. for 1.5 h. Then, the cooling bath was removed and the solution stirred at ambient temperature for 1.5 h. The reaction was quenched with water and extracted twice with EtOAc. The organic layers were combined, washed with water, brine, dried (MgSO₄) and concentrated in vacuo to afford a pale yellow solid as crude product. The residue was chromatographed on SiO₂ using a gradient of 15% EtOAc/hexanes to 35% EtOAc/hexanes to afford 2.17 g (20%) of product as a white solid. ESI-MS m/z 540 (MH)⁺.

Step 5. Synthesis of (1R)-1-(2-thiophene-2-yl-acetylamino)-2-(2-hydroxy-3-carboxyphenyl)ethyl-1-boronic acid. To a solution of (1R)-2-Methoxy-3-[2-(2-Thiophen-2-yl-acetylamino)-2-(2,9,9-trimethyl-3,5-dioxa-4-bora-tricyclo-[6.1.1.0^{2,6}]dec-4-yl)-ethyl]-benzoic acid isopropyl ester, (1.16 g, 21.48 mmole) in 1,4-dioxane (22 mL) was added 22 mL of 3N HCl. The mixture was heated to 110° C. and held for 90 min. The solution was cooled and diluted with 20 mL H₂O and extracted twice with diethyl ether (Et₂O). The aqueous layer was concentrated to afford a sticky residue as crude product. The residue was triturated with 5 mL H₂O to induce crystallization. The solids were washed twice with water and once with diethyl ether and dried in vacuo to afford 250 mg (33%) of the product as white powder. ESI-MS m/z 332 (MH-H₂O)⁺.

### Example 2

### (1R)-1-(2-thiophene-2-yl-acetylamino)-2-(2-hydroxy-4-carboxyphenyl)ethyl-1-boronic acid

Step 1, Synthesis of 4-Methoxy-3-(2,9,9-Trimethyl-3,5-dioxa-4-bora-tricyclo[6.1.1.0^{2,6}]dec-4-yl)-benzoic acid. A solution of (+)-pinanediol (8.7, 51.0 mmole) and 3-borono-4-methoxybenzoic acid (10.0 g, 51.2 mmole) in THF (70 mL) was stirred for 30 min at room temperature. The solution was concentrated in vacuo, and the residue was washed with hexanes to afford 15.1 g (89%) of the product as a slowly crystallizing white solid. ESI-MS m/z 331 (MH)⁺.

Step 2, Synthesis of 4-Methoxy-3-(2,9,9-Trimethyl-3,5-dioxa-4-bora-tricyclo[6.1.1.0^{2,6}]dec-4-yl)-benzoic acid isopropyl ester. A solution of 4-methoxy-3-(2,9,9-trimethyl-3,5-dioxa-4-bora-tricyclo[6.1.1.0^{2,6}]dec-4-yl)-benzoic acid (15.0 g, 45.4 mmole), 2-iodopropane (9.1 mL, d=1.7, 90.7 mmole) and potassium carbonate (12.6 g, 90.7 mmole) in DMF (220 mL) was stirred at ambient temperature for 18 h. The reaction was quenched with water and extracted twice with EtOAc. The combined organic layers were washed with water, brine, dried (MgSO₄) and concentrated in vacuo. The residue was chromatographed on SiO₂ using a gradient of 40% DCM/hexane to 90% DCM/hexane to afford 13.5 g (80%) of product as a white solid. ESI-MS m/z 373 (MH—C₄H₉)⁺.

Step 3. Synthesis of 4-Methoxy-3-(2,9,9-Trimethyl-3,5-dioxa-4-bora-tricyclo[6.1.1.0^{2,6}]dec-4-ylmethyl)-benzoic acid isopropyl ester. To a solution of 4-methoxy-3-(2,9,9-trimethyl-3,5-dioxa-4-bora-tricyclo[6.1.1.0^{2,6}]dec-4-yl)-benzoic acid isopropyl ester (10.6, 28.49 mmole) and chloroiodomethane (2.6 mL, 35.61 mmole) in THF (84 mL) at −100° C. was added n-BuLi (2.5M in hexanes, 14.2 mL, 35.61 mmole) over 6 minutes. The solution was stirred at −100° C. for 45 min, then the bath was removed and the solution stirred at ambient temperature for 15 h. The reaction was quenched with water and extracted twice with EtOAc. The combined organic layers were washed with water, brine, dried (Na₂SO₄) and concentrated in vacuo. The residue was chromatographed on SiO₂ using a gradient of 40% DCM/hexane to 70% DCM/hexane to afford 14.2 g (66%) of product as a colorless oil which contained 50% of the starting 4-methoxy-3-(2,9,9-trimethyl-3,5-dioxa-4-bora-tricyclo[6.1.1.0^{2,6}]dec-4-yl)-benzoic acid isopropyl ester. ESI-MS m/z 387 (MH)⁺.

Step 4. Synthesis of (1R)-4-Methoxy-3-[2-(2-Thiophen-2-yl-acetylamino)-2-(2,9,9-trimethyl-3,5-dioxa-4-bora-tricyclo[6.1.1.0^{2,6}]dec-4-yl)-ethyl]-benzoic acid isopropyl ester. To a solution of anhydrous dichloromethane (2.2 mL, 34.5 mmole) in THF (77 mL) at −100° C. was added n-BuLi (2.5M in hexanes, 10.4 mL, 25.8 mmole) over 15 min. The solution was stirred for 30 in at −100° C. at which point the microcrystalline LiCHCl₂ precipitate was visible. A solution of 4-methoxy-3-(2,9,9-trimethyl-3,5-dioxa-4-bora-tricyclo[6.1.1.0^{2,6}]dec-4-ylmethyl)-benzoic acid isopropyl ester (8.31 g, 21.5 mmole) in THF (16 mL) was added over 5 min by syringe. The mixture was stirred at −100° C. for 15 min, then warmed to 0° C. and held for 1 h. The solution was then cooled to −78° C. and a solution of LHMDS (1.0 M in THF, 25.8 mL, 25.8 mmole) was added over 5 min. The reaction was allowed to warm gradually while stirring overnight. The mixture was then cooled to −10° C. and anhydrous methanol (1.04 mL, 25.7 mmole) was added. This stirred for 45 min, then the bath was removed and the solution stirred at ambient temperature for 1.25 h. After cooling to −78° C., 2-thiopheneacetyl chloride (3.45 mL, 27.9 mmole) was added and the solution stirred at −78° C. for 15 min. The cooling bath was removed and the solution stirred at ambient temperature until complete. The reaction was quenched with water and extracted twice with EtOAc. The organic layers were combined, washed with water, brine, dried (MgSO₄) and concentrated in vacuo to afford a yellow solid as crude product. The residue was chromatographed on SiO₂ using a gradient of 15% EtOAc/hexane to 35% EtOAc/hexane to afford 570 mg (5%) of product as a pale yellow solid. ESI-MS m/z 540 (MH)⁺.

Step 5. Synthesis of (1R)-1-(2-thiophene-2-yl-acetylamino)-2-(6-hydroxy-3-carboxyphenyl)ethyl-1-boronic acid. To a solution of (1R)-4-Methoxy-3-[2-(2-Thiophen-2-yl-acetylamino)-2-(2,9,9-trimethyl-3,5-dioxa-4-bora-tricyclo[6.1.1.0^{2,6}]dec-4-yl)-ethyl]-benzoic acid isopropyl ester (150 mg, 0.28 mmole) in DCM (2.5 mL) was added 2.2 mL of 1M BBr₃ at −78° C. The reaction was allowed to warm gradually while stirring for 18 h. The reaction was quenched with 10 mL of H₂O. The solution was extracted twice with Et₂O. The aqueous layer was concentrated to afford a sticky residue as crude product. It was further purified by preparative HPLC to afford 8 mg (8%) of the product as an off-white powder. ESI-MS m/z 332 (MH-H₂O)⁺.

### Example 3

### (1R)-1-(3-hydroxy-phenyl)acetylamino-1-(3-carboxy-2-hydroxy)benzyl-methyl boronic acid

Step 1. Synthesis of 3-Borono-2-methoxybenzoic acid tert-butyl ester. To a solution of 3-borono-2-methoxybenzoic acid (Combi-blocks, 5.0 g, 25.5 mmole) in 1,4-dioxane (30 mL) in a sealed tube was added conc. H₂SO₄ (1.5 mL). The solution was cooled to 0° C. and an equal volume of 2-methylpropene was bubbled in. The tube was sealed and allowed to stir at ambient temperature for 18 h. The solution was cooled in an ice bath, the seal was opened and the solution stirred at ambient temperature for 30 min. The solution was basified with saturated aq. NaHCO₃ and extracted twice with EtOAc. The combined organic layers were washed with water (5×), brine, dried (Na₂SO₄) and concentrated in vacuo to afford 4.0 g (62%) of the product as a white solid. ESI-MS m/z 275 (M+Na)⁺.

Step 2. Synthesis of 2-Methoxy-3-(2,9,9-trimethyl-3,5-dioxa-4-bora-tricyclo[6.1.1.0^{2,6}]dec-4-yl)-benzoic acid tert-butyl ester. A solution of 3-Borono-2-methoxybenzoic acid tert-butyl ester (4.0 g, 15.9 mmole), THF (21 mL), and (+)-pinanediol (2.70 g, 15.9 mmole) was stirred at room temperature for 1 h. The solution was concentrated in vacuo, and the residue chromatographed on SiO₂ with 6% EtOAc/hexane to afford 5.0 g (86%) of the product as a slowly crystallizing solid. ESI-MS m/z 409 (M+Na)⁺.

Step 3. Synthesis of 2-Methoxy-3-(2,9,9-trimethyl-3,5-dioxa-4-bora-tricyclo [6.1.1.0^{2,6}]dec-4-ylmethyl)-benzoic acid tert-butyl ester. A solution of 2-Methoxy-3-(2,9,9-trimethyl-3,5-dioxa-4-bora-tricyclo[6.1.1.0^{2,6}]dec-4-yl)-benzoic acid tert-butyl ester (8.5 g, 22 mmol) and chloroiodomethane (4.6 g, 26.4 mmol) in THF (65 mL) under argon was cooled to −100° C. [MeOH, liq. N₂ slush bath]. n-BuLi (10.56 mL, 2.5M in hexane, 26.4 mmol) was added dropwise over a period of 10 minutes and the mixture stirred overnight. The reaction was quenched with H₂O (100 mL) and the aqueous phase was extracted with EtOAc (3×75 mL), the combined organic layers were dried over MgSO₄, and concentrated in vacuo. Purification by flash column chromatography on silica gel [R_{f}=0.21, (DCM/Hexane, 70:30, v/v)] afforded 8 g of the resultant compound as a colorless oil in 91% yield. ESI-MS m/z 401 (MH)⁺.

Step 4. Synthesis of (3-Benzyloxy-phenyl)-acetic acid benzyl ester. A mixture of 3-hydroxyphenylacetic acid (14.65 g, 96 mmole), benzyl bromide (27.4 mL, 231 mmole), potassium carbonate (39.9 g, 289 mmole) and dimethylformamide (DMF, 240 mL) was stirred at ambient temperature for 3 days. The reaction mixture was diluted with 1N NaOH and extracted twice with 50% EtOAC/hexanes. The combined organic layers were washed twice with 1N NaOH, water, brine, dried (Na₂SO₄) and concentrated in vacuo to afford 28.2 g (92%) of the product as a colorless oil which was used without further purification. ESI-MS m/z 319 (MH)⁺.

Step 5. Synthesis of 3-Benzyloxyphenylacetic acid. A solution of (3-benzyloxy-phenyl)-acetic acid benzyl ester (9.0 g, 27.1 mmole), 1N NaOH (84 mL, 84 mmole) and methanol (84 mL) was heated to 50° C. and stirred overnight. Water was added and the mixture extracted twice with Et₂O. The aqueous layer was acidified to pH 1 with concentrated HCl. The precipitated solids were collected by filtration, washed with water and dried to afford 5.34 g (79%) of the product as a white solid. ESI-MS m/z 243 (MH)⁺.

Step 6. Synthesis of 3-Benzyloxyphenylacetyl chloride. A solution of 3-benzyloxyphenylacetic acid (2.75 g, 11.4 mmole) in thionyl chloride (8.5 mL) was refluxed for 45 minutes, and the excess thionyl chloride was removed by distillation at atmospheric pressure and then the residual thionyl chloride was removed by adding chloroform three times and concentrating in vacuo each time.

Step 7. Synthesis of 3-[2-[2-(3-Benzyloxy-phenyl)-acetylamino]-2-(2,9,9-trimethyl-3,5-dioxa-4-bora-tricyclo[6.1.1.0^{2,6}]dec-4-yl)-ethyl]-2-methoxy-benzoic acid tert-butyl ester. To anhydrous CH₂Cl₂ (1.25 mL, 19.49 mmol) in anhydrous THF (55 mL) under argon at −100° C. [MeOH, liq. N₂ slush bath], n-BuLi (7.2 mL, 2.5M in hexane, 17.99 mmol) was added dropwise and the mixture was stirred for 30 minutes. A THF (10 mL) solution of 2-Methoxy-3-(2,9,9-trimethyl-3,5-dioxa-4-bora-tricyclo[6.1.1.0^{2,6}]dec-4-ylmethyl)-benzoic acid tert-butyl ester (6.0 g, 14.99 mmol) was added over a period of 20 minutes. After 40 minutes the cooling bath was removed and the mixture warmed slowly to 0° C. After 2 hours the reaction flask was cooled to −78° C., LHMDS (16.5 mL, 1M in THF, 16.5 mmol) was added slowly and the resultant solution was warmed to room temperature gradually while stirring overnight. Anhydrous MeOH (0.66 mL, 16.49 mmol) was added at −10° C., the reaction stirred for 1 h at the same temperature and then for 1 h at room temperature. 3-Benzyloxyphenylacetyl chloride (6.4 g, 24 mmol) was added at −78° C. and the mixture stirred for 40 minutes and allowed to reach room temperature. After 2.5 h the reaction was quenched with H₂O (75 mL) and the aqueous phase was extracted with EtOAc (3×75 mL), the combined organic layers were dried over MgSO₄, and concentrated in vacuo. Purification by flash column chromatography on silica gel [R_{f}=0.45, (EtOAc/Hexane, 40:60, v/v)] afforded 3.3 g of the resultant compound as pale yellow solid in 33% yield. ESI-MS m/z 654 (MH)⁺.

Step 8. Synthesis of (1R)-1-(3-hydroxy-phenyl)acetylamino-1-(3-carboxy-2-hydroxy)benzyl-methyl boronic acid. To a solution of 3-[2-[2-(3-Benzyloxy-phenylyacetylamino]-2-(2,9,9-trimethyl-3,5-dioxa-4-bora-tricyclo[6.1.1.0^{2,6}]dec-4-yl)-ethyl]-2-methoxy-benzoic acid tert-butyl ester (250 mg, 0.38 mmol) in dioxane (4 mL), 6N HCl (4 mL) was added dropwise at 110° C. Reaction progress was monitored by LC/MS for the disappearance of starting material. After 6 h H₂O (25 mL) was added and the mixture extracted with Et₂O (3×20 mL). The aqueous solution was concentrated in vacuo and purified by preparative HPLC to give 80 mg of resultant compound as a white solid in 58% yield. ESI-MS m/z 342 (MH-H₂O)⁺.

### Example 4

### (1R)-1-[3-(2-amino)-ethoxy-phenyl]acetylamino-1-(3-carboxy-2-hydroxy)benzyl-methylboronic acid hydrochloride

Step 1. Synthesis of 3-[2-[2-(3-Hydroxy-phenyl)-acetylamino]-2-(2,9,9-trimethyl-3,5-dioxa-4-bora-tricyclo[6.1.1.0^{2,6}]dec-4-yl)-ethyl]-2-methoxy-benzoic acid tert-butyl ester. To a solution of 3-[2-[2-(3-Benzyloxy-phenyl)-acetylamino]-2-(2,9,9-trimethyl-3,5-dioxa-4-bora-tricyclo[6.1.1.0^{2,6}]dec-4-yl)-ethyl]-2-methoxy-benzoic acid tert-butyl ester (1.24 g, 1.9 mmol) in MeOH (17 mL), Pd(OH)₂ (380 mg) was added and the mixture stirred under H₂ (45 psi) atmosphere for 5 h. The reaction mixture was filtered through CELITE® (diatomaceous earth) and concentrated in vacuo. Purification by flash column chromatography on silica gel [R_{f}=0.35, (EtOAc/Hexane, 40:60, v/v)] afforded 0.6 g of the resultant phenol as a colorless oil in 57% yield. ESI-MS m/z 564 (MH)⁺.

Step 2. Synthesis of 3-[2-{2-[3-(2-tert-Butoxycarbonylamino-ethoxy)-phenyl]-acetyl amino}-2-(2,9,9-trimethyl-3,5-dioxa-4-bora-tricyclo[6.1.1.0^{2,6}]dec-4-yl)-ethyl]-2-methoxy-benzoic acid tert-butyl ester. To a solution of 3-[2-[2-(3-Hydroxy-phenyl)-acetylamino]-2-(2,9,9-trimethyl-3,5-dioxa-4-bora-tricyclo[6.1.1.0^{2,6}]dec-4-yl)-ethyl]-2-methoxy-benzoic acid tert-butyl ester (280 mg, 0.5 mmol) in methylene chloride (5 mL) under argon was added triphenylphosphine (328 mg, 1.25 mmol) and N-boc-ethanolamine (0.19 mL, 1.25 mmol) and the mixture cooled to 0° C. Diisopropylazodicarbozylate (DIAD, 253 mg, 1.25 mmol) was added dropwise and the mixture stirred for 1 h. The ice bath was removed and the reaction stirred for another 4 h at room temperature. The reaction was quenched with H₂O (10 mL) and the aqueous phase was extracted with EtOAc (3×25 mL), the combined organic layers were dried over MgSO₄, and concentrated in vacuo. Purification by flash column chromatography on silica gel [R_{f}=0.25, (EtOAc/Hexane, 40:60, v/v)] afforded 180 mg of the resultant compound as colorless oil in 51% yield. ESI-MS m/z 708 (MH)⁺.

Step 3. Synthesis of (1R)-1-[3-(2-amino)-ethoxy-phenyl]acetylamino-1-(3-carboxy-2-hydroxy)benzyl-methylboronic acid hydrochloride. To a solution of 3-[2-{2-[3-(2-tert-Butoxycarbonylamino-ethoxy)-phenyl]-acetyl amino}-2-(2,9,9-trimethyl-3,5-dioxa-4-bora-tricyclo[6.1.1.0^{2,6}]dec-4-yl)-ethyl]-2-methoxy-benzoic acid tert-butyl ester (180 mg, 0.25 mmol) in dioxane (3 mL), 3N HCl (3 mL) was added dropwise at 110° C. Reaction progress was monitored by LC/MS for the disappearance of starting material. After 1 h, H₂O (20 mL) was added and the mixture extracted with Et₂O (3×20 mL). The aqueous solution was concentrated in vacuo and purified by preparative HPLC to give 15 mg of resultant compound as a white solid in 15% yield. ESI-MS m/z 385 (MH-H₂O)⁺.

### Example 5

### (1R)-1-(4-oxo-4-thiophen-2-yl-butyrylamino)-2-(2-hydroxy-3-carboxyphenyl)ethyl-1-boronic acid

Step 1. Preparation of carbonic acid isobutyl ester 4-Oxo-4-thiophen-2-yl-butyryl ester. To a solution of 4-oxo-4-(thiophen-2-yl)butanoic acid (2.57 g, 13.95 mmole) and 4-methylmorpholine (NMM, 1.7 mL, 15.4 mmole) in 14 mL of DCM at 0° C. was added isobutylchloroformate (1.8 mL, 13.95 mmole). The mixture was stirred for 45 min at 0° C. to complete the preparation of the mixed anhydride.

Step 2. Synthesis of (1R)-2-Methoxy-3-[2-(4-oxo-4-thiophen-2-yl-butyrylamino)-2-(2,9,9-trimethyl-3,5-dioxa-4-bora-tricyclo[6.1.1.0^{2,6}]dec-4-yl)-ethyl]-benzoic acid tert-butyl ester. To a solution of anhydrous dichloromethane (0.70 mL, 10.9 mmole) in THF (17 mL) at −100° C. was added n-BuLi (2.5M in hexanes, 3.4 mL, 8.4 mmole) over 15 min. The solution was stirred for 30 min at −100° C. at which point the microcrystalline LiCHCl₂ precipitate was visible. A solution of 2-methoxy-3-(2,9,9-trimethyl-3,5-dioxa-4-bora-tricyclo[6.1.1.0^{2,6}]dec-4-ylmethyl)-benzoic acid tert-butyl ester (2.8 g, 7.0 mmole) in THF (6 mL) was added over 5 min by syringe. The mixture was stirred at −100° C. for 15 min, then warmed to 0° C. and held for 2 h. The solution was then cooled to −78° C. and a solution of LHMDS (1.0 M in THF, 8.4 mL, 8.4 mmole) was added over 5 min. The reaction was allowed to warm gradually while stirring overnight. The mixture was then cooled to −10° C. and anhydrous methanol (0.33 mL, 8.4 mmole) was added. This stirred for 45 min, then the bath was removed and the solution stirred at ambient temperature for 1.25 h. After cooling to −78° C., the 0.5M carbonic acid isobutyl ester 4-Oxo-4-thiophen-2-yl-butyryl solution from Step 1 was added and the solution stirred at −78° C. for 15 min. The cooling bath was removed and the solution stirred at ambient temperature until completion. The reaction was quenched with water and extracted twice with EtOAc. The organic layers were combined, washed with water, brine, dried (MgSO₄) and concentrated in vacuo to afford a yellow solid as crude product. The residue was chromatographed on SiO₂ using a gradient of 25% EtOAc/hexanes to 40% EtOAc/hexanes to afford 514 mg (12%) of product as white solid. ESI-MS m/z 596 (MH)⁺.

Step 3. Synthesis of (1R)-1-(4-oxo-4-thiophen-2-yl-butyrylamino)-2-(2-hydroxy-3-carboxyphenyl)ethyl-1-boronic acid. To a solution of (1R)-2-Methoxy-3-[2-(4-oxo-4-thiophen-2-yl-butyrylamino)-2-(2,9,9-trimethyl-3,5-dioxa-4-bora-tricyclo[6.1.1.0^{2,6}]dec-4-yl)-ethyl]-benzoic acid tert-butyl ester, (510 mg, 0.85 mmole) in 1,4-dioxane (9 mL) was added 9 mL of 3N HCl. The mixture was heated to 110° C. and held for 90 min. The solution was cooled and diluted with 15 mL of H₂O, and extracted twice with Et₂O. The aqueous layer was concentrated to afford a sticky residue as crude product. The residue was triturated with 5 mL H₂O to induce crystallization. The solids were washed twice with water and once with Et₂O and then dried in vacuo to afford 120 mg (35%) of the product as a white powder. ESI-MS m/z 374 (MH-H₂O)⁺.

### Example 6

### (1R)-1-(2-acetylamino)-2-(2-hydroxy-3-carboxyphenyl)ethyl-1-boronic acid

Step 1. Synthesis of (1R)-2-Methoxy-3-[2-(2-acetylamino)-2-(2,9,9-trimethyl-3,5-dioxa-4-bora-tricyclo[6.1.1.0^{2,6}]dec-4-yl)-ethyl]-benzoic acid tert butyl ester. To a solution of anhydrous dichloromethane (0.64 mL, 8.6 mmole) in THF (19 mL) at −100° C. was added n-BuLi (2.5M in hexanes, 2.7 mL, 6.3 mmole) over 15 min. The solution was stirred for 30 min at −100° C. at which point the microcrystalline LiCHCl₂ precipitate was visible. A solution of 2-methoxy-3-(2,9,9-trimethyl-3,5-dioxa-4-bora-tricyclo[6.1.1.0^{2,6}]dec-4-ylmethyl)-benzoic acid tert-butyl ester (2.1 g, 5.25 mmole) in THF (4 mL) was added over 5 min by syringe. The mixture was stirred at −100° C. for 15 min, then warmed to 0° C. and held for 2 h. The solution was then cooled to −78° C. and a solution of LHMDS (1.0 M in THF, 5.3 mL, 5.3 mmole) was added over 5 min. The reaction was allowed to warm gradually while stirring overnight. The mixture was then cooled to −10° C. and anhydrous methanol (0.26 mL, 5.3 mmole) was added. This stirred for 45 min, then the bath was removed and the solution stirred at ambient temperature for 1.25 h. After cooling to −78° C., acetyl chloride (0.78 mL, 9.6 mmole) was added and the solution stirred at −78° C. for 1.5 h. Then, the cooling bath was removed and the solution stirred at ambient temperature for 1.5 h. The reaction was quenched with water and extracted twice with EtOAc. The organic layers were combined, washed with water, brine, dried (MgSO₄) and concentrated in vacuo to afford a pale yellow solid as crude product. The residue was chromatographed on SiO₂ using a gradient of 40% EtOAc/hexanes to 60% EtOAc/hexanes to afford 562 mg (23%) of product as a white solid. ESI-MS m/z 472 (MH)⁺.

Step 2. Synthesis of (1R)-1-(2-acetylamino)-2-(2-hydroxy-3-carboxyphenyl)ethyl-1-boronic acid. To a solution of (1R)-2-Methoxy-3-[2-(2-hydrogenacetylamino)-2-(2,9,9-trimethyl-3,5-dioxa-4-bora-tricyclo[6.1.1.0^{2,6}]dec-4-yl)-ethyl]-benzoic acid tert butyl ester, (370 mg, 0.78 mmole) in 1,4-dioxane (8 mL) was added 8 mL of 3N HCl. The mixture was heated to 110° C. and held for 90 min. The solution was cooled and diluted by 8 mL H₂O and extracted twice with Et₂O. The aqueous layer was concentrated to afford a sticky residue as crude product. The residue was further purified by preparative HPLC to afford 105 mg (50%) of the product as white powder. ESI-MS m/z 250 (MH-H₂O)⁺.

### Example 7

### (1R)-1-[3-(carboxymethoxy)-phenyl]acetylamino-(3-carboxy-2-hydroxy)benzyl-methylboronic acid

Step 1. Synthesis of 3-[2-[2-(3-Carbamoylmethoxy-phenyl)-acetylamino]-2-(2,9,9-trimethyl-3,5-dioxa-4-bora-tricyclo[6.1.1.0^{2,6}]dec-4-yl)-ethyl]-2-methoxy-benzoic acid tert-butyl ester. To a solution of 3-[2-[2-(3-Hydroxy-phenylyacetylamino]-2-(2,9,9-trimethyl-3,5-dioxa-4-bora-tricyclo[6.1.1.0^{2,6}]dec-4-yl)-ethyl]-2-methoxy-benzoic acid tert-butyl ester (610 mg, 1.08 mmol), prepared as described in Example 4, in DMF (5 mL) under argon was added K₂CO₃ (300 mg, 2.16 mmol). After stirring for 10 minutes, bromoacetamide (300 mg, 2.16 mmol) was added and the mixture stirred for 7 h at room temperature. The reaction was quenched with H₂O (20 mL) the aqueous phase was extracted with EtOAc (3×35 mL), the combined organic layers were dried over MgSO₄ and concentrated in vacuo. Purification by flash column chromatography on silica gel [R_{f}=0.15, (EtOAc/Hexane, 80:20, v/v)] afforded 309 mg of the resultant compound as a colorless oil in 46% yield. ESI-MS m/z 621 (MH)⁺.

Step 2 Synthesis of (1R)-1-[3-(carboxymethoxy)-phenyl]acetylamino-(3-carboxy-2-hydroxy)benzyl-methylboronic acid. To a solution of 3-[2-[2-(3-Carbamoylmethoxy-phenyl)-acetylamino]-2-(2,9,9-trimethyl-3,5-dioxa-4-bora-tricyclo[6.1.1.0^{2,6}]dec-4-yl)-ethyl]-2-methoxy-benzoic acid tert-butyl ester (70 mg, 0.11 mmol) in dioxane (3 mL), 3N HCl (3 mL) was added dropwise at 110° C. Reaction progress was monitored by LC/MS for the disappearance of starting material. After 1 h H₂O (20 mL) was added and the mixture extracted with Et₂O (3×20 mL). The aqueous solution was concentrated in vacuo and purified by preparative HPLC to give 14 mg of resultant compound as a white solid in 29% yield. ESI-MS m/z 400 (MH-H₂O)⁺.

### Example 8

### (1R)-1-[(3-carbamoylmethoxy)-phenyl]-acetylamino-(3-carboxy-2-hydroxy)benzyl-methylboronic acid

To a solution of 3-[2-[2-(3-Carbamoylmethoxy-phenyl)-acetylamino]-2-(2,9,9-trimethyl-3,5-dioxa-4-bora-tricyclo[6.1.1.0^{2,6}]dec-4-yl)-ethyl]-2-methoxy-benzoic acid tert-butyl ester (500 mg, 0.8 mmol) in DCM (9 mL), prepared as described in Example 7, BCl₃ (4.8 mL, 1M solution in DCM) was added dropwise at −78° C. The mixture was stirred for 1.5 h at the same temperature then warmed to room temperature. After 4 h the reaction was quenched with water (70 mL) and the mixture extracted with Et₂O (3×40 mL). The aqueous solution was concentrated in vacuo and purified by preparative HPLC to give 120 mg of resultant compound as a white solid in 37% yield. ESI-MS m/z 399 (MH-H₂O)⁺.

### Example 9

### (1R)-1-(2-(4-bromo-thiophen-2-nyl)acetylamino)-2-(2-hydroxy-3-carboxyphenyl)ethyl-1-boronic acid

Step 1. Synthesis of [2-(4-Bromo-thiophen-2-yl)-1-dimethylamino-vinyl]-methoxymethyl-phosphinic acid ethyl ester. To a suspension of sodium hydride (852 mg, 60% mineral oil dispersion, 22.2 mmol) in 34 mL of THF was added slowly a solution of tetraethyl dimethylaminomethylene diphosphonate (6.92 g, 20.93 mmol) in 34 mL of THF. After stirring 1 h, a solution of 4-bromo-2-thiophene carboxaldehyde (4 g, 20.94 mmol) in 34 mL of THF was added. The resulting mixture was heated at reflux for 1 h, then cooled to ambient temperature. The reaction mixture was partitioned between diethyl ether and water. The organic layer was washed sequentially with 1N HCl, water and brine, dried (MgSO₄) and concentrated. The crude product was further purified by flash column chromatography on SiO₂, eluting with a gradient of 15% EtOAc/hexanes to 25% EtOAc/hexanes to afford the titled compound of 2.4 g (31%) as a pale yellow solid. ESI-MS m/z 368 (MH)⁺.

Step 2. Synthesis of (4-Bromo-thiophen-2-yl)-acetic acid. A mixture of [2-(4-Bromo-thiophen-2-yl)-1-dimethylamino-vinyl]-methoxymethyl-phosphinic acid ethyl ester (2.4 g, 6.51 mmol) and 42 mL of 6N HCl was heated at reflux for 2 h. After cooling to ambient temperature, ice water was added and the mixture was partitioned between diethyl ether and water. The organic layer was washed with water twice, brine, dried (MgSO₄) and concentrated in vacuo to afford 1.30 g (91%) of the title compound.

Step 3. Synthesis of (4-Bromo-thiophen-2-yl)-acetyl chloride. A solution of 4-Bromo-thiophen-2-yl)-acetic acid (1.302 g, 5.9 mmole) in thionyl chloride (6 mL) was refluxed for 1 h. The solution was cooled and concentrated in vacuo to afford the acid chloride as a very sticky hard dark green oil.

Step 4. Synthesis of (1R)-1-(2-(4-bromothiophen-2-yl)acetylamino)-2-(2-hydroxy-3-carboxyphenyl)ethyl-1-boronic acid. This was prepared as described in Example 6 from 2-methoxy-3-(2,9,9-trimethyl-3,5-dioxa-4-bora-tricyclo[6.1.1.0^{2,6}]dec-4-ylmethyl)-benzoic acid tert-butyl ester and 2 eq of 4-Bromo-thiophen-2-yl)-acetyl chloride. The final product was further purified by preparative HPLC. ESI-MS m/z 410 (MH-H₂O)⁺.

### Example 10

### (1R)-1-(2-phenylacetylamino-2-(2-hydroxy-3-carboxyphenyl)ethyl-1-boronic acid

Prepared from 2-methoxy-3-(2,9,9-trimethyl-3,5-dioxa-4-bora-tricyclo[6.1.1.0^{2,6}]dec-4-ylmethyl)-benzoic acid tert-butyl ester and 1.3 eq of phenylacetyl chloride following the procedure described in Example 6. The final product was purified by preparative HPLC. ESI-MS m/z 326 (MH-H₂O)⁺.

### Example 11

### (1R)-1-(thiophene-2-carbonyl-amino)-2-(2-hydroxy-3-carboxyphenyl)ethyl-1-boronic acid

Prepared from 2-methoxy-3-(2,9,9-trimethyl-3,5-dioxa-4-bora-tricyclo[6.1.1.0^{2,6}]dec-4-ylmethyl)-benzoic acid tert-butyl ester and 1.3 eq of 2-thiophenecarbonyl chloride following the procedure described in Example 6. The final product was purified by preparative HPLC. ESI-MS m/z 318 (MH-H₂O)⁺.

### Example 12

### (1R,2′S)-1-(2-amino-2-phenylacetylamino)-2-(2-hydroxy-3-carboxyphenyl)ethyl-1-boronic acid formate salt

Step 1. Synthesis of (S)-tert-Butoxycarbonylamino-phenyl-acetic acid [1,2,3]triazolo[4,5-b]pyridin-3-yl ester. To a solution of L-Boc-α-phenylglycine (2.51 g, 10 mmole) in 16 mL of DCM at 0° C. was added N-methylmorpholine (NMM, 1.65 mL, 15 mmole) followed by O-(7-Azabenzotriazol-1-yl)-N,N,N′,N′-tetramethyluronium hexafluorophosphate (HATU, 3.81 g, 10 mmole). The solution was stirred for 30 min at 0° C. and then 30 min at ambient temperature. The solution was used as is for the acylation step.

Step 2. Synthesis of 3-[2-(2-tert-Butoxycarbonylamino-2-phenyl-acetylamino)-2-(2,9,9-trimethyl-3,5-dioxa-4-bora-tricyclo[6.1.1.0^{2,6}]dec-4-yl)-ethyl]-2-methoxy-benzoic acid tert-butyl ester. To a solution of anhydrous DCM (0.48 mL, 7.5 mmole) in THF (9 mL) at −100° C. was added n-BuLi (2.5 M in THF, 2.4 mL, 6.0 mmole) over 15 min. After stirring for 30 min at −100° C., a solution of 3-[2-(2-tert-Butoxycarbonylamino-2-phenyl-acetylamino)-2-(2,9,9-trimethyl-3,5-dioxa-4-bora-tricyclo[6.1.1.0^{2,6}]dec-4-yl)-ethyl]-2-methoxy-benzoic acid tert-butyl ester (2.0 g, 5.0 mmole) in THF (4 mL) was added over 4 min, and the transfer made quantitative with 2 mL THF. The solution was stirred for 5 min at −100° C. and then 30 min at 0° C. After cooling to −78° C., LHMDS (1.0 M in THF, 6.0 mL, 6.0 mmole) was added and the solution allowed to warm to room temperature slowly with stirring overnight. After cooling to −10° C., MeOH (0.245 mL, 6.0 mmole) was added and the solution stirred at −10° C. for 1 h then warmed to room temperature and held for an additional hour. The solution was then cooled to −10° C. and the solution of (S)-tert-Butoxycarbonylamino-phenyl-acetic acid [1,2,3]triazolo[4,5-b]pyridin-3-yl ester from Step 1 was added in one portion. The cooling bath was removed and the solution allowed to stir overnight. The reaction was quenched with water and extracted twice with EtOAc. The combined organic layers were washed with 1N HCl, water, brine, dried (Na₂SO₄) and concentrated in vacuo. The residue was chromatographed on silica gel to afford 470 mg (14%) of product as a yellow foam. ESI-MS m/z 663 (MH)⁺.

Step 3. Synthesis of (1R,2′S)-1-(2-amino-2-phenylacetylamino)-2-(2-hydroxy-3-carboxyphenyl)ethyl-1-boronic acid formate salt. To a solution of 3-[2-(2-tert-Butoxycarbonylamino-2-phenyl-acetylamino)-2-(2,9,9-trimethyl-3,5-dioxa-4-bora-tricyclo[6.1.1.0^{2,6}]dec-4-yl)-ethyl]-2-methoxy-benzoic acid tert-butyl ester (220 mg, 0.33 mmole) in DCM (1.0 mL) at −78° C. was added BCl₃ (1M in DCM, 3.0 mL, 3.0 mmole). The cooling bath was removed and the solution stirred at ambient temperature for 3 h. The reaction was quenched with water and extracted three times with ether. The aqueous layer was concentrated in vacuo, and the residue purified by preparative HPLC using solvents buffered with 0.1% formic acid to afford 19.4 mg (16%) of the product as a white solid. ESI-MS m/z 341 (MH-H₂O)⁺.

### Example 13

### (1R)-1-Benzoylamino-1-(3-carboxy-2-hydroxy)benzyl-methylboronic acid

Step 1 Synthesis of (1R)-1-benzoylamino-1-(3-tert-butoxycarbonyl-2-methoxy)benzyl-methylboronate. Prepared from 2-Methoxy-3-(2,9,9-trimethyl-3,5-dioxa-4-bora-tricyclo[6.1.1.0^{2,6}]dec-4-ylmethyl)-benzoic acid tert-butyl ester and benzoyl chloride following the procedure described in Step 7 of Example 3. The crude product was purified by flash column chromatography [R_{f}=0.2, silica gel (EtOAc/Hexane, 30:70, v/v)] to give a 30% yield of product. ESI-MS m/z 534 (MH)⁺.

Step 2. Synthesis of (1R)-1-Benzoylamino-1-(3-carboxy-2-hydroxy)benzyl-methylboronic acid. To a solution of 3-[2-Benzoylamino-2-(2,9,9-trimethyl-3,5-dioxa-4-bora-tricyclo[6.1.1.0^{2,6}]dec-4-yl)-ethyl]-2-methoxy-benzoic acid tert-butyl ester (330 mg, 0.62 mmol) in dioxane (6 mL), 3N HCl (6 mL) was added dropwise at 110° C. After 1 h H₂O (40 mL) was added and the mixture extracted with Et₂O (3×30 mL). The aqueous solution was concentrated in vacuo and purified by preparative HPLC to give 20 mg of resultant compound as a white solid in 10% yield. ESI-MS m/z 312 (MH-H₂O)⁺.

### Example 14

### (1R)-1-Isobutyrylamino-1-(3-carboxy-2-hydroxy)benzyl-methylboronic acid

Step 1 Synthesis of 3-[2-Isobutyrylamino-2-(2,9,9-trimethyl-3,5-dioxa-4-bora-tricyclo[6.1.1.0^{2,6}]dec-4-yl)-ethyl]-2-methoxy-benzoic acid tert-butyl ester. Prepared from 2-Methoxy-3-(2,9,9-trimethyl-3,5-dioxa-4-bora-tricyclo[6.1.1.0^{2,6}]dec-4-ylmethyl)-benzoic acid tert-butyl ester and isobutyryl chloride following the procedure described in Step 7 of Example 3. The crude product was purified by flash column chromatography [R_{f}=0.25, silica gel (EtOAc/Hexane, 40:60, v/v)] to afford a 21% yield of product. ESI-MS m/z 500 (MH)⁺.

Step 2. Synthesis of (1R)-1-Isobutyrylamino-1-(3-carboxy-2-hydroxy)benzyl-methylboronic acid. To a solution of 3-[2-Isobutyrylamino-2-(2,9,9-trimethyl-3,5-dioxa-4-bora-tricyclo[6.1.1.0^{2,6}]dec-4-yl)-ethyl]-2-methoxy-benzoic acid tert-butyl ester (225 mg, 0.45 mmol) in dioxane (5 mL), 3N HCl (5 mL) was added dropwise at 110° C. After 1 h H₂O (40 mL) was added and the mixture extracted with Et₂O (3×30 mL). The aqueous solution was concentrated in vacuo and purified by preparative HPLC to give 60 mg of resultant compound as a white solid in 48% yield. ESI-MS m/z 278 (MH-H₂O)⁺.

### Example 15

### (1R)-1-Cyclopentanecarbonylamino-(3-carboxy-2-hydroxy)benzyl-methylboronic acid

Step 1. Synthesis of 3-[2-(Cyclopentanecarbonyl-amino)-2-(2,9,9-trimethyl-3,5-dioxa-4-bora-tricyclo[6.1.1.0^{2,6}]dec-4-yl)-ethyl]-2-methoxy-benzoic acid tert-butyl ester. Prepared from 2-Methoxy-3-(2,9,9-trimethyl-3,5-dioxa-4-bora-tricyclo[6.1.1.0^{2,6}]dec-4-ylmethyl)-benzoic acid tert-butyl ester and cyclopentanecarbonyl chloride following the procedure described in Step 7 of Example 3. The crude product was purified by flash column chromatography [R_{f}=0.15, silica gel (EtOAc/Hexane, 30:70, v/v)] to afford the product in 25% yield. ESI-MS m/z 526 (MH)⁺.

Step 2. Synthesis of (1R)-1-Cyclopentanecarbonylamino-(3-carboxy-2-hydroxy)benzyl-methylboronic acid. To a solution of 3-[2-(Cyclopentanecarbonyl-amino)-2-(2,9,9-trimethyl-3,5-dioxa-4-bora-tricyclo[6.1.1.0^{2,6}]dec-4-yl)-ethyl]-2-methoxy-benzoic acid tert-butyl ester (260 mg, 0.49 mmol) in dioxane (5 mL), 3N HCl (5 mL) was added dropwise at 110° C. After 1 h H₂O (40 mL) was added and the mixture extracted with Et₂O (3×30 mL). The aqueous solution was concentrated in vacuo and purified by preparative HPLC to give 40 mg of resultant compound as a white solid in 28% yield. ESI-MS m/z 304 (MH-H₂O)⁺.

### Example 16

### (1R)-1-(propionylamino)-2-(2-hydroxy-3-carboxyphenyl)ethyl-1-boronic acid

Prepared from 2-methoxy-3-(2,9,9-trimethyl-3,5-dioxa-4-bora-tricyclo[6.1.1.0^{2,6}]dec-4-ylmethyl)-benzoic acid tert-butyl ester and 1.3 eq of propionyl bromide following the procedure described in Example 6. The final product was purified by preparative. ESI-MS m/z 264 (MH-H₂O)⁺.

### Example 17

### (1R)-1-(2-(2,5-dimethoxyphenyl)-acetylamino)-2-(2-hydroxy-3-carboxyphenyl)ethyl-1-boronic acid

Prepared from 2-methoxy-3-(2,9,9-trimethyl-3,5-dioxa-4-bora-tricyclo[6.1.1.0^{2,6}]dec-4-ylmethyl)-benzoic acid tert-butyl ester and 1.3 eq of 2,5-Dimethoxyphenylacetyl chloride following the procedure described in Example 6. ESI-MS m/z 386 (MH-H₂O)⁺.

### Example 18

### (1R)-1-(2-(2,5-dihydroxyphenyl)acetylamino)-2-(2-hydroxy-3-carboxyphenyl)ethyl-1-boronic acid

Step 1. Synthesis of (1R)-2-Methoxy-3-[2-[2-(2,5-dimethoxyphenyl)acetylamino]-2-(2,9,9-trimethyl-3,5-dioxa-4-bora-tricyclo[6.1.1.0^{2,6}]dec-4-yl)-ethyl]benzoic acid tert butyl ester. Prepared from 2-methoxy-3-(2,9,9-trimethyl-3,5-dioxa-4-bora-tricyclo[6.1.1.0^{2,6}]dec-4-ylmethyl)-benzoic acid tert-butyl ester and 1.3 eq of 2,5-Dimethoxyphenylacetyl chloride following the procedure described in Example 6. ESI-MS m/z 608 (MH)⁺.

Step 2. Synthesis of (1R)-1-(2-(2,5-dihydroxyphenyl)acetylamino)-2-(2-hydroxy-3-carboxyphenyl)ethyl-1-boronic acid. To a solution of (1R)-2-Methoxy-3-[2-[2-(2,5-dimethoxyphenyl)acetylamino]-2-(2,9,9-trimethyl-3,5-dioxa-4-bora-tricyclo[6.1.1.0^{2,6}]dec-4-yl)-ethyl]benzoic acid tert-butyl ester (249 mg, 0.4 mmol) in DCM (4 mL), BBr₃ (4.2 mL, 1M solution in DCM) was added dropwise at −78° C. The mixture was stirred while the temperature was slowly warmed up to ambient temperature. After 4.5 h, the reaction was quenched with water (10 mL) and the mixture extracted with Et₂O (3×10 mL). The aqueous solution was concentrated in vacuo and purified by preparative HPLC to afford 40 mg (26%) of the product as a white solid. ESI-MS m/z 358 (MH-H₂O)⁺.

### Example 19

### (1R)-1-Hydroxyacetylamino-1-(3-carboxy-2-hydroxy)benzyl-methyl boronic acid

Step 1. Synthesis of 3-[2-(2-Acetoxy-acetylamino)-2-(2,9,9-trimethyl-3,5-dioxa-4-bora-tricyclo[6.1.1.0^{2,6}]dec-4-yl)-ethyl]-2-methoxy-benzoic acid tert-butyl ester. Prepared from 2-Methoxy-3-(2,9,9-trimethyl-3,5-dioxa-4-bora-tricyclo[6.1.1.0^{2,6}]dec-4-ylmethyl)-benzoic acid tert-butyl ester and acetoxyacetyl chloride following the procedure described in Step 7 of Example 3. The crude product was purified by flash column chromatography [R_{f}=0.2, silica gel (EtOAc/Hexane, 40:60, v/v)] to afford the product in 10% yield. ESI-MS m/z 530 (MH)⁺.

Step 2. Synthesis of (1R)-1-Hydroxyacetylamino-1-(3-carboxy-2-hydroxy)benzyl-methylboronic acid. To a solution of 3-[2-(2-Acetoxy-acetylamino)-2-(2,9,9-trimethyl-3,5-dioxa-4-bora-tricyclo[6.1.1.0^{2,6}]dec-4-yl)-ethyl]-2-methoxy-benzoic acid tert-butyl ester (80 mg, 0.15 mmol) in dioxane (2 ml), 3N HCl (2 ml) was added dropwise at 110° C. After 1 h H₂O (20 ml) was added and the mixture extracted with Et₂O (3×30 ml). The aqueous solution was concentrated in vacuo and purified by preparative HPLC to give 9 mg of resultant compound as a white solid in 22% yield. ESI-MS m/z 266 (MH-H₂O)⁺.

### Example 20

### (1R)-1-Cyclopropanecarbonyl-amino-1-(3-carboxy-2-hydroxy)benzyl-methylboronic acid

Step 1. Synthesis of 3-[2-(Cyclopropanecarbonyl-amino)-2-(2,9,9-trimethyl-3,5-dioxa-4-bora-tricyclo[6.1.1.0^{2,6}]dec-4-yl)-ethyl]-2-methoxy-benzoic acid tert-butyl ester. Prepared from 2-Methoxy-3-(2,9,9-trimethyl-3,5-dioxa-4-bora-tricyclo[6.1.1.0^{2,6}]dec-4-ylmethyl)-benzoic acid tert-butyl ester and cyclopropylcarbonyl chloride following the procedure described in Step 7 of Example 3. The crude product was purified by flash column chromatography [R_{f}=0.16, silica gel (EtOAc/Hexane, 40:60, v/v)] to afford the product in 22% yield. ESI-MS m/z 498 (MH)⁺.

Step 2. Synthesis of (1R)-1-Cyclopropanecarbonylamino-1-(3-carboxy-2-hydroxy)benzyl-methylboronic acid. To a solution of 3-[2-(Cyclopropanecarbonyl-amino)-2-(2,9,9-trimethyl-3,5-dioxa-4-bora-tricyclo[6.1.1.0^{2,6}]dec-4-yl)ethyl]-2-methoxy-benzoic acid tert-butyl ester (177 mg, 0.35 mmol) in dioxane (4 ml), 3N HCl (4 ml) was added dropwise at 110° C. After 1 h H₂O (25 ml) was added and the mixture extracted with Et₂O (3×30 ml). The aqueous solution was concentrated in vacuo and purified by preparative HPLC to give 20 mg of resultant compound as a white solid in 20% yield. ESI-MS m/z 276 (MH-H₂O)⁺.

### Example 21

### (1R)-1-Hexanoylamino-(3-carboxy-2-hydroxy)benzyl-methylboronic acid

Step 1. Synthesis of 3-[2-(Hexanoylamino)-2-(2,9,9-trimethyl-3,5-dioxa-4-bora-tricyclo[6.1.1.0^{2,6}]dec-4-yl)-ethyl]-2-methoxy-benzoic acid tert-butyl ester. Prepared from 2-Methoxy-3-(2,9,9-trimethyl-3,5-dioxa-4-bora-tricyclo[6.1.1.0^{2,6}]dec-4-ylmethyl)-benzoic acid tert-butyl ester and hexanoyl chloride following the procedure described in Step 7 of Example 3. The crude product was purified by flash column chromatography [R_{f}=0.18, silica gel (EtOAc/Hexane, 40:60, v/v)] to afford the product in 22% yield. ESI-MS m/z 528 (MH)⁺.

Step 2. Synthesis of (1R)-1-Hexanoylamino-(3-carboxy-2-hydroxy)benzyl-methylboronic acid. To a solution of 3-[2-Hexanoylamino-2-(2,9,9-trimethyl-3,5-dioxa-4-bora-tricyclo[6.1.1.0^{2,6}]dec-4-yl)-ethyl]-2-methoxy-benzoic acid tert-butyl ester (175 mg, 0.33 mmol) in dioxane (4 ml), 3N HCl (4 ml) was added dropwise at 110° C. After 1 h H₂O (25 ml) was added and the mixture extracted with Et₂O (3×30 ml). The aqueous solution was concentrated in vacuo and purified by preparative HPLC to give 14 mg of resultant compound as a white solid in 14% yield. ESI-MS m/z 306 (MH-H₂O)⁺.

### Example 22

### (1R)-1-(2-benzyloxyacetylamino)-2-(2-hydroxy-3-carboxyphenyl)ethyl-1-boronic acid

Prepared from 2-methoxy-3-(2,9,9-trimethyl-3,5-dioxa-4-bora-tricyclo[6.1.1.0^{2,6}]dec-4-ylmethyl)-benzoic acid tert-butyl ester and 1.3 eq of benzyloxyacetyl chloride following the procedure described in Example 6. The final product was purified by preparative HPLC. ESI-MS m/z 356 (MH-H₂O)⁺.

### Example 23

### (1R)-1-(pentanoylamino)-2-(2-hydroxy-3-carboxyphenyl)ethyl-1-boronic acid

Prepared from 2-methoxy-3-(2,9,9-trimethyl-3,5-dioxa-4-bora-tricyclo[6.1.1.0^{2,6}]dec-4-ylmethyl)-benzoic acid tert-butyl ester and 1.3 eq of valeryl chloride following the procedure described in Example 6. The final product was purified by preparative HPLC. ESI-MS m/z 292 (MH-H₂O)⁺.

### Example 24

### (1R)-1-(heptanoylamino)-2-(2-hydroxy-3-carboxyphenyl)ethyl-1-boronic acid

Prepared from 2-methoxy-3-(2,9,9-trimethyl-3,5-dioxa-4-bora-tricyclo[6.1.1.0^{2,6}]dec-4-ylmethyl)-benzoic acid tert-butyl ester and 1.3 eq of heptanoyl chloride following the procedure described in Example 6. The final product was purified by preparative HPLC. ESI-MS m/z 320 (MH-H₂O)⁺.

### Example 25

### (1R)-1-(3,3-Dimethyl-butyryl amino)-(3-carboxy-2-hydroxy)benzyl-methylboronic acid

Prepared from 2-methoxy-3-(2,9,9-trimethyl-3,5-dioxa-4-bora-tricyclo[6.1.1.0^{2,6}]dec-4-ylmethyl)-benzoic acid tert-butyl ester and 3,3-dimethylbutyryl chloride following the procedure described in Steps 7 and 8 of Example 3. The final product was purified by preparative HPLC. ESI-MS m/z 306 (MH-H₂O)⁺.

### Example 26

### (1R)-1-(4-Fluorobenzoylamino)-(3-carboxy-2-hydroxy)benzyl-methylboronic acid

Prepared from 2-methoxy-3-(2,9,9-trimethyl-3,5-dioxa-4-bora-tricyclo[6.1.1.0^{2,6}]dec-4-ylmethyl)-benzoic acid tert-butyl ester and 4-fluorobenzoyl chloride following the procedure described in Steps 7 and 8 of Example 3. The final product was purified by preparative HPLC. ESI-MS m/z 330 (MH-H₂O)⁺.

### Example 27

### (1R)-1-(1-naphthoyl amino)-2-(2-hydroxy-3-carboxyphenyl)ethyl-1-boronic acid

Prepared from 2-methoxy-3-(2,9,9-trimethyl-3,5-dioxa-4-bora-tricyclo[6.1.1.0^{2,6}]dec-4-ylmethyl)-benzoic acid tert-butyl ester and 1.3 eq of 1-naphthoyl chloride following the procedure described in Example 6. The final product was purified by preparative HPLC. ESI-MS m/z 362 (MH-H₂O)⁺.

### Example 28

### (1R)-1-(3-hydroxybenzoyl amino)-2-(2-hydroxy-3-carboxyphenyl)ethyl-1-boronic acid

Prepared from 2-methoxy-3-(2,9,9-trimethyl-3,5-dioxa-4-bora-tricyclo[6.1.1.0^{2,6}]dec-4-ylmethyl)-benzoic acid tert-butyl ester and 1.3 eq of 3-methoxybenzoyl chloride following the procedure described in Example 18. The final product was purified by preparative HPLC. ESI-MS m/z 328 (MH-H₂O)⁺.

### Example 29

### (1R)-1-(3-methoxybenzoyl amino)-2-(2-hydroxy-3-carboxyphenyl)ethyl-1-boronic acid

Prepared from 2-methoxy-3-(2,9,9-trimethyl-3,5-dioxa-4-bora-tricyclo[6.1.1.0^{2,6}]dec-4-ylmethyl)-benzoic acid tert-butyl ester and 1.3 eq of 3-methoxybenzoyl chloride following the procedure described in Example 6. The final product was purified by preparative HPLC. ESI-MS m/z 342 (MH-H₂O)⁺.

### Example 30

### (1R)-1-(2-methylbenzoyl amino)-2-(2-hydroxy-3-carboxyphenyl)ethyl-1-boronic acid

Prepared from 2-methoxy-3-(2,9,9-trimethyl-3,5-dioxa-4-bora-tricyclo[6.1.1.0^{2,6}]dec-4-ylmethyl)-benzoic acid tert-butyl ester and 1.3 eq of 2-methylbenzoyl chloride following the procedure described in Example 6. The final product was purified by preparative HPLC. ESI-MS m/z 326 (MH-H₂O)⁺.

### Example 31

### (1R)-1-(6-Chloro-pyridine-3-carbonyl)-amino-1-(3-carboxy-2-hydroxy)benzyl-methylboronic acid

Step 1 Synthesis of 3-[2-[(6-Chloro-pyridine-3-carbonyl)-amino]-2-(2,9,9-trimethyl-3,5-dioxa-4-bora-tricyclo[6.1.1.0^{2,6}]dec-4-yl)-ethyl]-2-methoxy-benzoic acid tert-butyl ester. Prepared from 2-Methoxy-3-(2,9,9-trimethyl-3,5-dioxa-4-bora-tricyclo[6.1.1.0^{2,6}]dec-4-ylmethyl)-benzoic acid tert-butyl ester and 6-chloro-nicotinoyl chloride following the procedure described in Step 7 of Example 3. The crude product was purified by flash column chromatography [R_{f}=0.18, silica gel (EtOAc/Hexane, 30:70, v/v)] to give a 24% yield of product. ESI-MS m/z 569 (MH)⁺.

Step 2. Synthesis of (1R)-1-(6-Chloro-pyridine-3-carbonyl)-amino-1-(3-carboxy-2-hydroxy)benzyl-methylboronic acid. To a solution of 3-[2-[(6-Chloro-pyridine-3-carbonyl)-amino]-2-(2,9,9-trimethyl-3,5-dioxa-4-bora-tricyclo[6.1.1.0^{2,6}]dec-4-yl)-ethyl]-2-methoxy-benzoic acid tert-butyl ester (400 mg, 0.7 mmol) in dioxane (9 mL), 3N HCl (9 mL) was added dropwise at 110° C. After 1 h H₂O (40 mL) was added and the mixture extracted with Et₂O (3×40 mL). The aqueous solution was concentrated in vacuo and purified by preparative HPLC to give 9 mg of resultant compound as a white solid in 4% yield. ESI-MS m/z 347 (MH-H₂O)⁺.

### Example 32

### (1R)-1-(4-Chloro-benzoyl)-amino-1-(3-carboxy-2-hydroxy)benzyl-methylboronic acid

Step 1 Synthesis of 3-[2-(4-Chloro-benzoylamino)-2-(2,9,9-trimethyl-3,5-dioxa-4-bora-tricyclo[6.1.1.0^{2,6}]dec-4-yl)-ethyl]-2-methoxy-benzoic acid tert-butyl ester. Prepared from 2-Methoxy-3-(2,9,9-trimethyl-3,5-dioxa-4-bora-tricyclo[6.1.1.0^{2,6}]dec-4-ylmethyl)-benzoic acid tert-butyl ester and 4-chloro-benzoyl chloride following the procedure described in Step 7 of Example 3. The crude product was purified by flash column chromatography [R_{f}=0.33, silica gel (EtOAc/Hexane, 30:70, v/v)] to give a 28% yield of product. ESI-MS m/z 568 (MH)⁺.

Step 2. (1R)-1-(4-Chloro-benzoyl)-amino-1-(3-carboxy-2-hydroxy)benzyl-methyl boronic acid. To a solution of 3-[2-(4-Chloro-benzoylamino)-2-(2,9,9-trimethyl-3,5-dioxa-4-bora-tricyclo[6.1.1.0^{2,6}]dec-4-yl)-ethyl]-2-methoxy-benzoic acid tert-butyl ester (640 mg, 1.12 mmol) in dioxane (11 ml), 3N HCl (11 ml) was added dropwise at 110° C. After 1 hr H₂O (25 ml) was added and the mixture extracted with Et₂O (3×400 ml). The aqueous solution was concentrated in vacuo and purified by analytical HPLC to give 13 mg of resultant compound as a white solid in 3% yield. ESI-MS m/z 346 (MH-H₂O)⁺.

### Example 33

### (1R)-1-(4-Methoxybenzoyl)-amino-1-(3-carboxy-2-hydroxy)benzyl-methylboronic acid

Step 1. Synthesis of 2-Methoxy-3-[2-(4-methoxybenzoylamino)-2-(2,9,9-trimethyl-3,5-dioxa-4-bora-tricyclo[6.1.1.0^{2,6}]dec-4-yl)-ethyl]-benzoic acid tert-butyl ester. Prepared from 2-Methoxy-3-(2,9,9-trimethyl-3,5-dioxa-4-bora-tricyclo[6.1.1.0^{2,6}]dec-4-ylmethyl)-benzoic acid tert-butyl ester and 4-methoxybenzoyl chloride following the procedure described in Step 7 of Example 3. The crude product was purified by flash column chromatography [R_{f}=0.22, silica gel (EtOAc/Hexane, 40:60, v/v)] to give a 26% yield of product. ESI-MS m/z 564 (MH)⁺.

Step 2. (1R)-1-(4-Methoxybenzoyl)-amino-1-(3-carboxy-2-hydroxy)benzyl-methyl boronic acid. To a solution of 2-Methoxy-3-[2-(4-methoxybenzoylamino)-2-(2,9,9-trimethyl-3,5-dioxa-4-bora-tricyclo[6.1.1.0^{2,6}]dec-4-yl)-ethyl]-benzoic acid tert-butyl ester (225 mg, 0.4 mmol) in dioxane (4 ml), 3N HCl (4 ml) was added dropwise at 110° C. After 1 hr H₂O (25 ml) was added and the mixture extracted with Et₂O (3×30 ml). The aqueous solution was concentrated in vacuo and purified by analytical HPLC to give 16 mg of resultant compound as a white solid in 11% yield. ESI-MS m/z 342 (MH-H₂O)⁺.

### Example 34

### (1R)-1-(2-methoxybenzoyl amino)-2-(2-hydroxy-3-carboxyphenyl)ethyl-1-boronic acid

Prepared from 2-methoxy-3-(2,9,9-trimethyl-3,5-dioxa-4-bora-tricyclo[6.1.1.0^{2,6}]dec-4-ylmethyl)-benzoic acid tert-butyl ester and 1.3 eq of 2-methoxybenzoyl chloride following the procedure described in Example 6. The final product was purified by preparative HPLC. ESI-MS m/z 342 (MH-H₂O)⁺.

### Example 35

### (1R)-1-(2-hydroxybenzoyl amino)-2-(2-hydroxy-3-carboxyphenyl)ethyl-1-boronic acid

Prepared from 2-methoxy-3-(2,9,9-trimethyl-3,5-dioxa-4-bora-tricyclo[6.1.1.0^{2,6}]dec-4-ylmethyl)-benzoic acid tert-butyl ester and 1.3 eq of 2-methoxybenzoyl chloride following the procedure described in Example 18. The final product was purified by preparative HPLC. ESI-MS m/z 328 (MH-H₂O)⁺.

### Example 36

### (1R)-1-(4-Hydroxybenzoyl)-amino-1-(3-carboxy-2-hydroxy)benzyl-methylboronic acid

To a solution of 2-Methoxy-3-[2-(4-methoxybenzoylamino)-2-(2,9,9-trimethyl-3,5-dioxa-4-bora-tricyclo[6.1.1.0^{2,6}]dec-4-yl)-ethyl]-benzoic acid tert-butyl ester (225 mg, 0.4 mmol), prepared as described in Example 33, in DCM (9 mL), BBr₃ (2.4 mL, 1M solution in DCM) was added dropwise at −78° C. The mixture was stirred for 2 h at the same temperature then warmed to room temperature. After 4 h the reaction was quenched with water (50 mL) and the mixture extracted with Et₂O (3×40 mL). The aqueous solution was concentrated in vacuo and purified by preparative HPLC to give 15 mg of resultant compound as a white solid in 11% yield. ESI-MS m/z 328 (MH-H₂O)⁺.

### Example 37

### (1R)-1-(2-acetamidoacetylamino)-2-(2-hydroxy-3-carboxyphenyl)ethyl-1-boronic acid

Prepared from 2-methoxy-3-(2,9,9-trimethyl-3,5-dioxa-4-bora-tricyclo[6.1.1.0^{2,6}]dec-4-ylmethyl)-benzoic acid tert-butyl ester and 2 eq of N-acetylglycine following the procedure described in Steps 1, 2 and 3 of Example 12 while the reaction temperature was kept at −78° C. for 2 h in Step 3. The final product was purified by preparative HPLC. ESI-MS m/z 307 (MH-H₂O)⁺.

### Example 38

### (1R)-1-(3-aminopropanoylamino)-2-(2-hydroxy-3-carboxyphenyl)ethyl-1-boronic acid formate salt

Prepared from 2-methoxy-3-(2,9,9-trimethyl-3,5-dioxa-4-bora-tricyclo[6.1.1.0^{2,6}]dec-4-ylmethyl)-benzoic acid tert-butyl ester and 2 eq of Boc-beta-alanine following the procedure described in Steps 1, 2 and 3 of Example 12 except that the reaction temperature for Step 3 was allowed to gradually warm from −78° C. to −30° C. over 2 hours before quenching. The final product was obtained after purification by preparative HPLC using solvents buffered with 0.1% formic acid. ESI-MS m/z 279 (MH-H₂O)⁺.

### Example 39

### (1R)-1-(2-Amino-thiazol-4-yl)-acetylamino-1-(3-carboxy-2-hydroxy)benzyl-methylboronic acid formate salt

Step 1. Synthesis of [2-(Trityl-amino)-thiazol-4-yl]-acetic acid methyl ester. A solution of methyl-2-amino-4-thiazoleacetic acid (3.0 g, 17.4 mmole), diisopropylethylamine (3.0 mL, 17.2 mmole), and trityl chloride (5.3 g, 19.0 mmole) in 70 mL of DCM was stirred for 4 days. Water was added, the layers separated and the aqueous layer extracted once with DCM. The organic layers were combined, washed twice with water, brine, dried (Na₂SO₄) and concentrated in vacuo. The crude product was chromatographed on SiO₂ using a gradient of 10% EtOAc/hexane to 40% EtOAc/hexane to afford 6.45 g (89%) of the title product as a white solid.

Step 2. Synthesis of [2-(Trityl-amino)-thiazol-4-yl]-acetic acid. A solution of [2-(Trityl-amino)-thiazol-4-yl]-acetic acid methyl ester (3.0 g, 7.25 mmole), methanol (50 mL) and 1N aqueous NaOH (20 mL) was stirred at ambient temperature for 23 h. During this time the solution went from a slurry to homogeneous. Water was added and the solution extracted twice with Et₂O. The aqueous layer was acidified to pH 1 with 3N HCl resulting in a white precipitate. The solids were collected by filtration, washed with water and dried in vacuo to afford 2.32 g (80%) of the title product as a white solid.

Step 3. Synthesis of 3-[2-[2-(2-Amino-thiazol-4-yl)-acetylamino]-2-(2,9,9-trimethyl-3,5-dioxa-4-bora-tricyclo[6.1.1.0^{2,6}]dec-4-yl)-ethyl]-2-methoxy-benzoic acid tert-butyl ester. To anhydrous CH₂Cl₂ (1.4 mL, 21.8 mmol) in anhydrous THF (53 mL) under argon at −100° C. [MeOH, liq. N₂ slush bath], n-BuLi (8.1 mL, 2.5M in hexane, 22.2 mmol) was added dropwise and the mixture was stirred for 30 minutes. A THF (12 mL) solution of 2-Methoxy-3-(2,9,9-trimethyl-3,5-dioxa-4-bora-tricyclo[6.1.1.0^{2,6}]dec-4-ylmethyl)-benzoic acid tert-butyl ester (6.73 g, 16.81 mmol) was added over a period of 20 minutes. After 40 minutes the cooling bath was removed and the mixture warmed slowly to 0° C. After 2 hours the reaction flask was cooled to −78° C., LHMDS (18.5 mL, 1M in THF, 18.5 mmol) was added slowly and the resultant solution was warmed to room temperature gradually while stirring overnight. Anhydrous MeOH (0.75 mL, 16.49 mmol) was added at −10° C., the reaction stirred for 1 h at the same temperature and then for 1 h at room temperature. At this stage LCMS indicated the formation of 2-Methoxy-3-[2-(2,9,9-trimethyl-3,5-dioxa-4-bora-tricyclo[6.1.1.0^{2,6}]dec-4-yl)-2-(trimethylsilanyl-amino)-ethyl]-benzoic acid tert-butyl ester intermediate.

In a separate dry round bottom flask under argon containing [2-(Trityl-amino)-thiazol-4-yl]-acetic acid, dry DCM (50 ml) was added. The content in the flask were cooled to 0° C. NMM (1.6 mL, 15.12 mmol) was added followed by HATU (4.1 g, 10.8 mmol) and the mixture stirred for 30 min at 0° C. and then 1 hr at room temperature. To this reaction mixture was added 30 mL of the solution from Step 1 dropwise at −20° C. The cooling bath was removed and the reaction stirred at room temperature. After 2 h the reaction was quenched with H₂O (75 mL) and the aqueous phase was extracted with EtOAc (3×75 mL), the combined organic layers were dried over MgSO₄, and concentrated in vacuo. Purification by flash column chromatography on silica gel [R_{f}=0.5, (EtOAc/Hexane, 60:40, v/v)] afforded 0.88 g of the resultant compound as pale yellow solid in 19% yield. ESI-MS m/z 812 (MH)⁺.

Step 4. Synthesis of (1R)-1-(2-Amino-thiazol-4-yl)-acetylamino-1-(3-carboxy-2-hydroxy)benzyl-methylboronic acid formate salt. Prepared from 2-Methoxy-3-(2-(2,9,9-trimethyl-3,5-dioxa-4-bora-tricyclo[6.1.1.0^{2,6}]dec-4-yl)-2-{2-[2-(trityl-amino)-thiazol-4-yl]-acetylamino}-ethyl)-benzoic acid tert-butyl ester and BCl₃, following the procedure described in Example 8. The crude product was purified by preparative HPLC using solvents buffered with formic acid to give 10 mg of resultant compound as a white solid in 5% yield. ESI-MS m/z 348 (MH-H₂O)⁺.

### Example 40

### (1R)-1-(Pyrazol-1-yl-acetyl amino)-2-(2-hydroxy-3-carboxyphenyl)ethyl-1-boronic acid

Prepared from 2-methoxy-3-(2,9,9-trimethyl-3,5-dioxa-4-bora-tricyclo[6.1.1.0^{2,6}]dec-4-ylmethyl)-benzoic acid tert-butyl ester and 2 eq of 2-(1H-pyrazol-1-yl)acetic acid following the procedure described in Steps 1, 2 and 3 of Example 12 while the reaction temperature was kept at −78° C. for 2 h in Step 3. The final product was obtained after purification by preparative HPLC. ESI-MS m/z 316 (MH-H₂O)⁺.

### Example 41

### (1R)-1-(2-aminoacetylamino)-2-(2-hydroxy-3-carboxyphenyl)ethyl-1-boronic acid formate salt

Prepared from 2-methoxy-3-(2,9,9-trimethyl-3,5-dioxa-4-bora-tricyclo[6.1.1.0^{2,6}]dec-4-ylmethyl)-benzoic acid tert-butyl ester and 2 eq of Boc-glycine following the procedure described in Example 12 except that the reaction temperature for Step 3 was allowed to gradually warm from −78° C. to −30° C. over 2 hours before quenching. The final product was obtained after purification by preparative HPLC using solvents buffered with 0.1% formic acid. ESI-MS m/z 265 (MH-H₂O)⁺.

### Example 42

### (1R)-1-(3-aminomethyl)-benzoylamino-1-(3-carboxy-2-hydroxy)benzyl-methyl boronic acid formate

Step 1 Synthesis of 3-[2-[3-(tert-Butoxycarbonylamino-methyl)-benzoylamino]-2-(2,9,9-trimethyl-3,5-dioxa-4-bora-tricyclo[6.1.1.0^{2,6}]dec-4-yl)-ethyl]-2-methoxy-benzoic acid tert-butyl ester. Prepared from 2-Methoxy-3-(2,9,9-trimethyl-3,5-dioxa-4-bora-tricyclo[6.1.1.0^{2,6}]dec-4-ylmethyl)-benzoic acid tert-butyl ester and 3-(tert-Butoxycarbonylaminomethyl)-benzoic acid following the procedure described in Step 3 of Example 39. The crude product was purified by flash column chromatography [R_{f}=0.26, silica gel (EtOAc/Hexane, 30:70, v/v)] to give a 22% yield of product. ESI-MS m/z 663 (MH)⁺.

Step 2. Synthesis of (1R)-1-(3-aminomethyl)-benzoylamino-1-(3-carboxy-2-hydroxy)benzyl-methyl boronic acid formate. Prepared from 3-[2-[3-(tert-Butoxycarbonylamino-methyl)-benzoylamino]-2-(2,9,9-trimethyl-3,5-dioxa-4-bora-tricyclo[6.1.1.0^{2,6}]dec-4-yl)-ethyl]-2-methoxy-benzoic acid tert-butyl ester and BCl₃, following the procedure described in Example 8. The crude product was purified by preparative HPLC using solvents buffered with 0.1% formic acid to give 10 mg of resultant compound as a white solid in 4% yield. ESI-MS m/z 341 (MH-H₂O)⁺.

### Example 43

### (1R)-1-(2,6-Dichlorobenzoyl)-amino-1-(3-carboxy-2-hydroxy)benzyl-methylboronic acid

Step 1 Synthesis of 3-[2-(2,6-Dichlorobenzoylamino)-2-(2,9,9-trimethyl-3,5-dioxa-4-bora-tricyclo[6.1.1.0^{2,6}]dec-4-yl)-ethyl]-2-methoxy-benzoic acid tert-butyl ester. Prepared from 2-Methoxy-3-(2,9,9-trimethyl-3,5-dioxa-4-bora-tricyclo[6.1.1.0^{2,6}]dec-4-ylmethyl)-benzoic acid tert-butyl ester and 2,6-dichlorobenzoyl chloride following the procedure described in Step 7 of Example 3. The crude product was purified by flash column chromatography [R_{f}=0.33, silica gel (EtOAc/Hexane, 30:70, v/v)] to give a 17% yield of product. ESI-MS m/z 603 (MH)⁺.

Step 2. Synthesis of (1R)-1-(2,6-Dichloro-benzoyl)-amino-1-(3-carboxy-2-hydroxy)benzyl-methyl boronic acid. To a solution of 3-[2-(2,6-Dichlorobenzoylamino)-2-(2,9,9-trimethyl-3,5-dioxa-4-bora-tricyclo[6.1.1.0^{2,6}]dec-4-yl)-ethyl]-2-methoxy-benzoic acid tert-butyl ester (350 mg, 0.58 mmol) in DCM (9 mL), BCl₃ (3.5 mL, 1 M solution in DCM) was added dropwise at −78° C. The mixture was stirred for 2 h at the same temperature then warmed to room temperature. After 4 h the reaction was quenched with water (50 mL) and the mixture extracted with Et₂O (3×40 mL). The aqueous solution was concentrated in vacuo and purified by preparative HPLC to give 6 mg of resultant compound as a white solid in 4% yield. ESI-MS m/z 381 (MH-H₂O)⁺.

### Example 44

### (1R)-1-(1-methyl-3-phenyl-1H-pyrazole-5-carbonyl-amino)-2-(2-hydroxy-3-carboxyphenyl)ethyl-1-boronic acid

Prepared from 2-methoxy-3-(2,9,9-trimethyl-3,5-dioxa-4-bora-tricyclo[6.1.1.0^{2,6}]dec-4-ylmethyl)-benzoic acid tert-butyl ester and 1.3 eq of 1-methyl-3-phenyl-1H-pyrazole-5-carbonyl chloride following the procedure described in Example 18 except that BCl₃ was used in place of BBr₃ and the reaction temperature for the final step was allowed to gradually warm from −78° C. to −30° C. over 2 hours before quenching. The final product was further purified by preparative HPLC. ESI-MS m/z 392 (MH-H₂O)⁺.

### Example 45

### (1R)-1-(2-(1,3-dioxoisoindolin-2-yl)acetyl amino)-2-(2-hydroxy-3-carboxyphenyl)ethyl-1-boronic acid

Prepared from 2-methoxy-3-(2,9,9-trimethyl-3,5-dioxa-4-bora-tricyclo[6.1.1.0^{2,6}]dec-4-ylmethyl)-benzoic acid tert-butyl ester and 2 eq of N-phthaloylglycine following the procedure described in Example 12 while the reaction temperature was kept at −78° C. for 2 h in Step 3. The final product was further purified by preparative HPLC. ESI-MS m/z 395 (MH-H₂O)⁺.

### Example 46

### (1R)-1-(isoxazole-5-carbonyl-amino)-2-(2-hydroxy-3-carboxyphenyl)ethyl-1-boronic acid

Prepared from 2-methoxy-3-(2,9,9-trimethyl-3,5-dioxa-4-bora-tricyclo[6.1.1.0^{2,6}]dec-4-ylmethyl)-benzoic acid tert-butyl ester and 1.3 eq of isoxazole 5-carbonyl chloride following the procedure described in Example 18 except that BCl₃ was used in place of BBr₃ and reaction temperature was kept at −78° C. for 2 h for the final step. The final product was purified by preparative HPLC. ESI-MS m/z 303 (MH-H₂O)⁺.

### Example 47

### (1R)-1-[3-(5-methyl-[1,2,4]oxadiazol-3-yl)-benzoyl]-amino-1-(3-carboxy-2-hydroxy)benzyl-methylboronic acid

Step 1. Synthesis of 2-Methoxy-3-[2-[3-(5-methyl-[1,2,4]oxadiazol-3-yl)-benzoyl amino]-2-(2,9,9-trimethyl-3,5-dioxa-4-bora-tricyclo[6.1.1.0^{2,6}]dec-4-yl)-ethyl]-benzoic acid tert-butyl ester. Prepared from 2-Methoxy-3-(2,9,9-trimethyl-3,5-dioxa-4-bora-tricyclo[6.1.1.0^{2,6}]dec-4-ylmethyl)-benzoic acid tert-butyl ester and 3-(5-Methyl-[1,2,4]oxadiazol-3-yl)-benzoic acid following the procedure described in Step 1 of Example 39. The crude product was purified by flash column chromatography [R_{f}=0.23, silica gel (EtOAc/Hexane, 40:60, v/v)] to give a 15% yield of product. ESI-MS m/z 616 (MH)⁺.

Step 2. (1R)-1-[3-(5-methyl-[1,2,4]oxadiazol-3-yl)-benzoylamino]-1-(3-carboxy-2-hydroxy)benzyl-methylboronic acid. Prepared from 2-Methoxy-3-[2-[3-(5-methyl-[1,2,4]oxadiazol-3-yl)-benzoylamino]-2-(2,9,9-trimethyl-3,5-dioxa-4-bora-tricyclo[6.1.1.0^{2,6}]dec-4-yl)-ethyl]-benzoic acid tert-butyl ester and BCl₃, following the procedure described in Example 8. The crude product was purified by preparative HPLC to give 8 mg of resultant compound as a white solid in 3% yield. ESI-MS m/z 394 (MH-H₂O)⁺.

### Example 48

### (1R)-1-(6-morpholin-4-yl-pyridine-3-carbonyl)-amino-1-(3-carboxy-2-hydroxy)benzyl-methylboronic acid

Step 1. Synthesis of 2-Methoxy-3-[2-[(6-morpholin-4-yl-pyridine-3-carbonyl)-amino]-2-(2,9,9-trimethyl-3,5-dioxa-4-bora-tricyclo[6.1.1.0^{2,6}]dec-4-yl)-ethyl]-benzoic acid tert-butyl ester. Prepared from 2-Methoxy-3-(2,9,9-trimethyl-3,5-dioxa-4-bora-tricyclo[6.1.1.0^{2,6}]dec-4-ylmethyl)-benzoic acid tert-butyl ester and 6-Morpholin-4-yl-nicotinoyl chloride following the procedure described in Step 7 of Example 3. The crude product was purified by flash column chromatography [R_{f}=0.21, silica gel (EtOAc/Hexane, 70:30, v/v)] to giver a 27% yield of product. ESI-MS m/z 620 (MH)⁺.

Step 2. (1R)-1-(6-morpholin-4-yl-pyridine-3-carbonyl)-amino-1-(3-carboxy-2-hydroxy)benzyl-methylboronic acid. Prepared from 2-Methoxy-3-[2-[(6-morpholin-4-yl-pyridine-3-carbonyl)-amino]-2-(2,9,9-trimethyl-3,5-dioxa-4-bora-tricyclo[6.1.1.0^{2,6}]dec-4-yl)-ethyl]-benzoic acid tert-butyl ester and BCl₃, following the procedure described in Example 8. The crude product was purified by preparative HPLC to give 80 mg of resultant compound as a white solid in 30% yield. ESI-MS m/z 398 (MH-H₂O)⁺.

### Example 49

### (1R)-1-(1-Acetyl-piperidine-4-carbonyl)-amino-1-(3-carboxy-2-hydroxy)benzyl-methylboronic acid

Step 1. Synthesis of 3-[2-[(1-Acetyl-piperidine-4-carbonyl)-amino]-2-(2,9,9-trimethyl-3,5-dioxa-4-bora-tricyclo[6.1.1.0^{2,6}]dec-4-yl)-ethyl]-2-methoxy-benzoic acid tert-butyl ester. Prepared from 2-Methoxy-3-(2,9,9-trimethyl-3,5-dioxa-4-bora-tricyclo[6.1.1.0^{2,6}]dec-4-ylmethyl)-benzoic acid tert-butyl ester and 1-Acetyl-piperidine-4-carboxylic acid following the procedure described in Step 7 of Example 3. The crude product was purified by flash column chromatography [R_{f}=0.12, silica gel (MeOH/CH₂Cl₂, 2:98, v/v)] to give a 18% yield of product. ESI-MS m/z 583 (MH)⁺.

Step 2. (1R)-1-(1-Acetyl-piperidine-4-carbonyl)-amino-1-(3-carboxy-2-hydroxy)benzyl-methylboronic acid. Prepared from 3-[2-[(1-Acetyl-piperidine-4-carbonyl)-amino]-2-(2,9,9-trimethyl-3,5-dioxa-4-bora-tricyclo[6.1.1.0^{2,6}]dec-4-yl)-ethyl]-2-methoxy-benzoic acid tert-butyl ester and BCl₃, following the procedure described in Example 8. The crude product was purified by preparative HPLC to give 58 mg of resultant compound as a white solid in 25% yield. ESI-MS m/z 361 (MH-H₂O)⁺.

Exemplary compounds of the present invention are shown in Table 1 along with respective molecular weights (MW) and low-resolution electrospray ionization mass spectral analytical results (ESI Mass Spec).

TABLE 1
Examples of compounds of the present invention.

| | | | | | | | | | ESI |
|---|---|---|---|---|---|---|---|---|---|
| | | | | | | | | | Mass |
| | | | | | | | | | Spec |
| Example | R1 | R2 | R3 | X1 | X2 | Y1 | Y2 | MW | (m/z) |
| 1 | 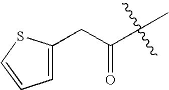 | H | 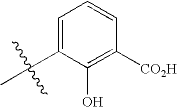 | OH | OH | H | H | 349 | 332 (MH − H₂O)+ |
| 2 | 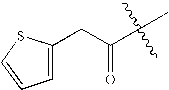 | H | 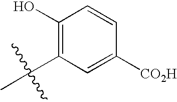 | OH | OH | H | H | 349 | 332 (MH − H₂O)+ |
| 3 | 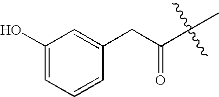 | H | 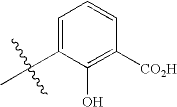 | OH | OH | H | H | 359 | 342 (MH − H₂O)+ |
| 4 | 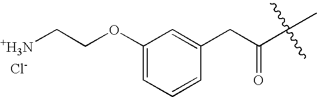 | H | 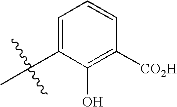 | OH | OH | H | H | 439 | 385 (MH − H₂O)+ |
| 5 | 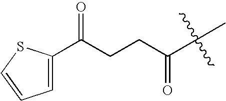 | H | 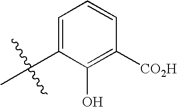 | OH | OH | H | H | 391 | 374 (MH − H₂O)+ |
| 6 | 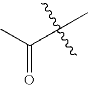 | H | 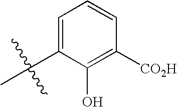 | OH | OH | H | H | 267 | 250 (MH − H₂O)+ |
| 7 | 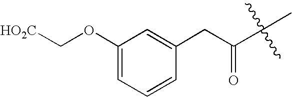 | H | 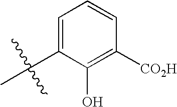 | OH | OH | H | H | 417 | 400 (MH − H₂O)+ |
| 8 | 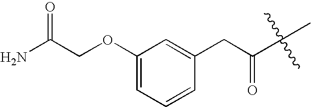 | H | 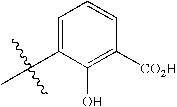 | OH | OH | H | H | 416 | 399 (MH − H₂O)+ |
| 9 | 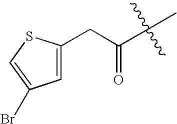 | H | 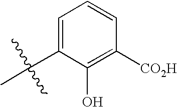 | OH | OH | H | H | 428 | 410 (MH − H₂O)+; 412 ((M + 2)H − H₂O) |
| 10 | 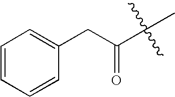 | H | 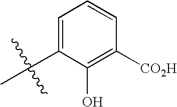 | OH | OH | H | H | 343 | 326 (MH − H₂O)+ |
| 11 | 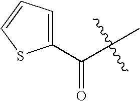 | H | 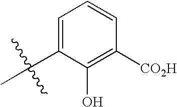 | OH | OH | H | H | 335 | 318 (MH − H₂O)+ |
| 12 | 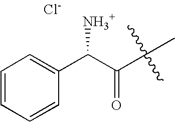 | H | 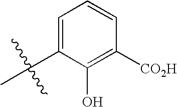 | OH | OH | H | H | 395 | 341 (MH − H₂O)+ |
| 13 | 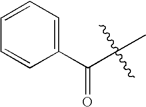 | H | 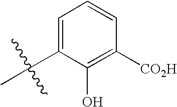 | OH | OH | H | H | 329 | 312 (MH − H₂O)+ |
| 14 | 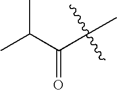 | H | 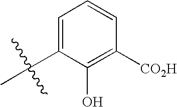 | OH | OH | H | H | 295 | 278 (MH − H₂O)+ |
| 15 | 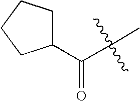 | H | 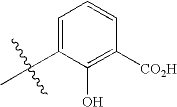 | OH | OH | H | H | 321 | 304 (MH − H₂O)+ |
| 16 | 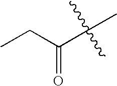 | H | 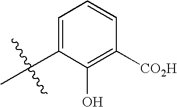 | OH | OH | H | H | 281 | 264 (MH − H₂O)+ |
| 17 | 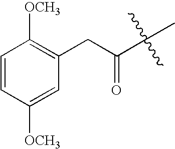 | H | 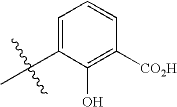 | OH | OH | H | H | 403 | 386 (MH − H₂O)+ |
| 18 | 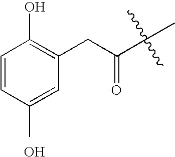 | H | 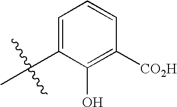 | OH | OH | H | H | 375 | 358 (MH − H₂O)+ |
| 19 | 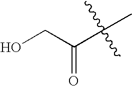 | H | 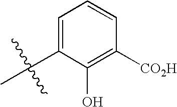 | OH | OH | H | H | 283 | 266 (MH − H₂O)+ |
| 20 | 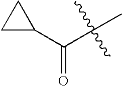 | H | 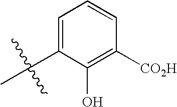 | OH | OH | H | H | 293 | 276 (MH − H₂O)+ |
| 21 | 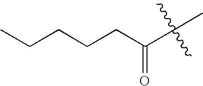 | H | 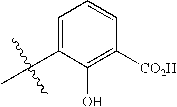 | OH | OH | H | H | 323 | 306 (MH − H₂O)+ |
| 22 | 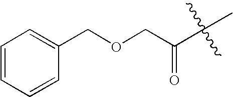 | H | 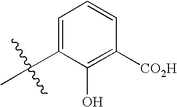 | OH | OH | H | H | 373 | 356 (MH − H₂O)+ |
| 23 | 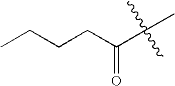 | H | 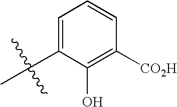 | OH | OH | H | H | 309 | 292 (MH − H₂O)+ |
| 24 | 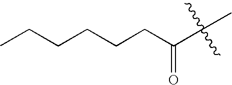 | H | 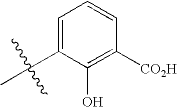 | OH | OH | H | H | 337 | 320 (MH − H₂O)+ |
| 25 | 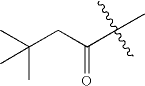 | H | 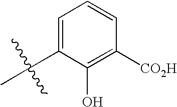 | OH | OH | H | H | 323 | 306 (MH − H₂O)+ |
| 26 | 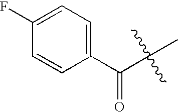 | H | 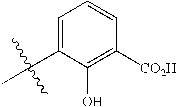 | OH | OH | H | H | 347 | 332 (MH − H₂O)+ |
| 27 | 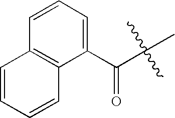 | H | 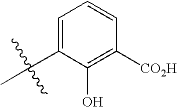 | OH | OH | H | H | 379 | 362 (MH − H₂O)+ |
| 28 | 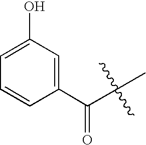 | H | 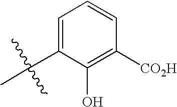 | OH | OH | H | H | 345 | 328 (MH − H₂O)+ |
| 29 | 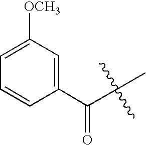 | H | 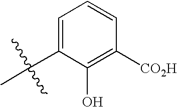 | OH | OH | H | H | 359 | 342 (MH − H₂O)+ |
| 30 | 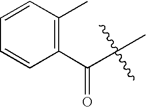 | H | 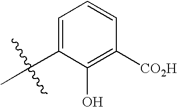 | OH | OH | H | H | 343 | 326 (MH − H₂O)+ |
| 31 | 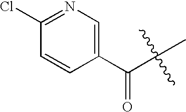 | H | 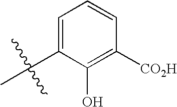 | OH | OH | H | H | 365 | 347 (MH − H₂O)+ |
| 32 | 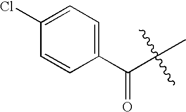 | H | 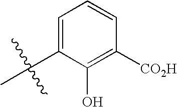 | OH | OH | H | H | 364 | 346 (MH − H₂O)+ |
| 33 | 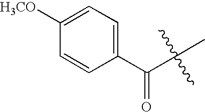 | H | 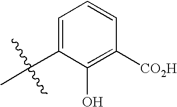 | OH | OH | H | H | 359 | 342 (MH − H₂O)+ |
| 34 | 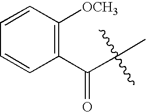 | H | 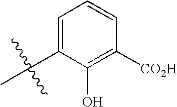 | OH | OH | H | H | 359 | 342 (MH − H₂O)+ |
| 35 | 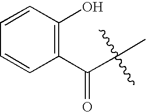 | H | 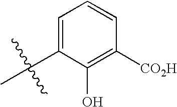 | OH | OH | H | H | 345 | 328 (MH − H₂O)+ |
| 36 | 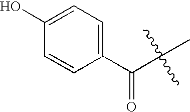 | H | 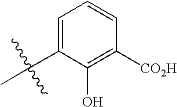 | OH | OH | H | H | 345 | 328 (MH − H₂O)+ |
| 37 | 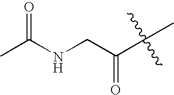 | H | 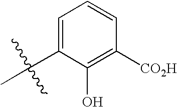 | OH | OH | H | H | 324 | 307 (MH − H₂O)+ |
| 38 | 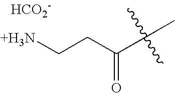 | H | 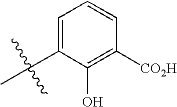 | OH | OH | H | H | 342 | 279 (MH − H₂O)+ |
| 39 | 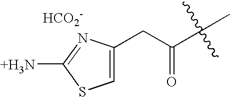 | H | 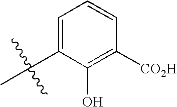 | OH | OH | H | H | 402 | 348 (MH − H₂O)+ |
| 40 | 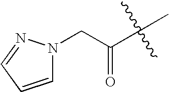 | H | 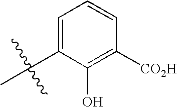 | OH | OH | H | H | 333 | 316 (MH − H₂O)+ |
| 41 | 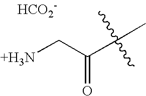 | H | 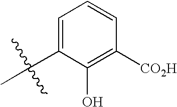 | OH | OH | H | H | 328 | 265 (MH − H₂O)+ |
| 42 | 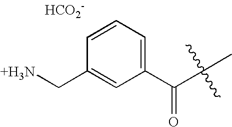 | H | 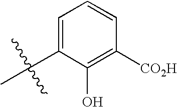 | OH | OH | H | H | 404 | 341 (MH − H₂O)+ |
| 43 | 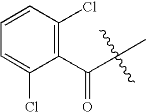 | H | 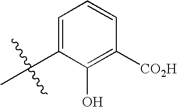 | OH | OH | H | H | 398 | 380 (MH − H₂O)+ |
| 44 | 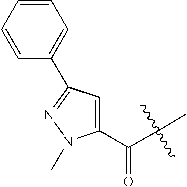 | H | 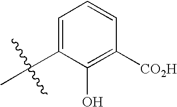 | OH | OH | H | H | 409 | 392 (MH − H₂O)+ |
| 45 | 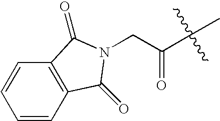 | H | 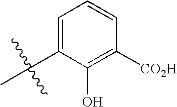 | OH | OH | H | H | 412 | 395 (MH − H₂O)+ |
| 46 | 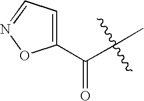 | H | 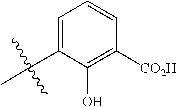 | OH | OH | H | H | 320 | 303 (MH − H₂O)+ |
| 47 | 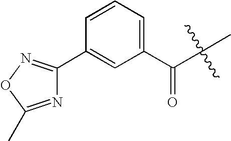 | H | 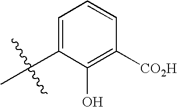 | OH | OH | H | H | 411 | 394 (MH − H₂O)+ |
| 48 | 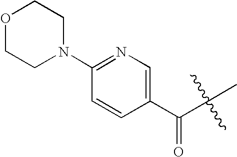 | H | 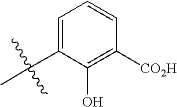 | OH | OH | H | H | 415 | 398 (MH − H₂O)+ |
| 49 | 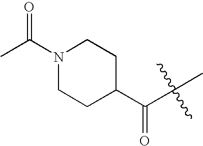 | H | 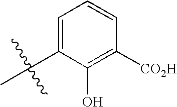 | OH | OH | H | H | 378 | 361 (MH − H₂O)+ |

### Example 50

### Experimental Method for β-Lactamase Enzyme Assays

Isolation of β-lactamases. Crude β-lactamase extracts were prepared from 20 ml overnight cultures with shaking. Escherichia coli cells containing SHV-5 or CTXM-15, Enterobacter cloacae cells containing P99, and Klebsiella pneumoniae cells containing KPC-2 were further diluted 10-fold and grown to mid-log phase (OD at 600 nm, 0.5-0.8) in Mueller-Hinton II (MH-II) broth at 37° C. The cells were pelleted at 5000 g, washed and resuspended in 2 mL PBS pH 7.0. The β-lactamases were extracted by four cycles of freezing and thawing followed by centrifugation. β-lactamase activity in the extracts was measured with the chromogenic cephalosporin nitrocefin. The amount of protein in each β-lactamase preparation was determined by the bicinchoninic acid (BCA) assay.

β-lactamase Inhibition. To determine the level of inhibition of β-lactamase enzymes, compounds were diluted in PBS at pH 7.0 to yield concentrations between 100 and 0.005 μM in microtiter plates. An equal volume of diluted enzyme stock was added, and the plates were incubated at 37° C. for 10 min. Nitrocefin solution was then dispensed as substrate into each well at a final concentration of 100 μM, and the plates were immediately read with the kinetic program at 486 nm for 10 min on the SPECTRAMAX® Plus³⁸⁴ (high-throughput microplate spectrophotometer; Molecular Devices Corp., Sunnyvale, Calif.). Maximum rates of metabolism were then compared to those in control wells (without inhibitors), and percentages of enzyme inhibition were calculated for each concentration of inhibitor. The concentration of inhibitor needed to reduce the initial rate of hydrolysis of substrate by 50% (IC₅₀) was calculated as the residual activity of β-lactamase at 486 nm using the SoftMax Pro 5.0 software (Molecular Devices Corp.).

Using the methodology described above, examples of the current invention were evaluated for their ability to inhibit β-lactamase enzymes. The results of these assays are summarized in Table 2 for representative enzymes across different subtypes (note SHV-5 and CTXM-15 exemplify different subclasses of Ambler Class A Extended Spectrum Beta Lactamases, KPC-2 exemplifies Class A carbapenemases, and P99 represents chromosomal Class C AmpC), where A represents an IC₅₀ of >1 μM, B represents an IC₅₀ of 0.1 to 1 μM, C represents an IC₅₀ of 0.01 to 0.1 μM, and D represents an IC₅₀ of <0.01 μM. NT=Not tested.

TABLE 2
Inhibition of diverse β-lactamases by example compounds
of the present invention.

| | SHV-5 IC₅₀ | CTXM-15 IC₅₀ | P99 AmpC | KPC-2 IC₅₀ |
|---|---|---|---|---|
| Example | Range | Range | IC₅₀ Range | Range |
| 1 | D | D | D | D |
| 2 | C | B | B | NT |
| 3 | D | D | D | NT |
| 4 | D | D | D | NT |
| 5 | D | D | D | NT |
| 6 | B | D | C | NT |
| 7 | D | D | D | NT |
| 8 | D | D | D | NT |
| 9 | D | D | D | NT |
| 10 | D | D | D | NT |
| 11 | C | D | D | NT |
| 12 | B | C | B | NT |
| 13 | C | C | D | NT |
| 14 | B | C | D | NT |
| 15 | B | C | D | NT |
| 16 | B | C | C | C |
| 17 | D | D | D | D |
| 18 | D | D | D | D |
| 19 | B | C | B | C |
| 20 | B | D | D | C |
| 21 | D | D | D | D |
| 22 | D | D | C | D |
| 23 | C | D | C | D |
| 24 | D | D | D | C |
| 25 | C | C | C | C |
| 26 | C | D | D | D |
| 27 | C | D | D | D |
| 28 | C | D | D | C |
| 29 | C | D | D | D |
| 30 | C | D | D | D |
| 31 | D | D | D | D |
| 32 | C | D | D | D |
| 33 | B | D | D | D |
| 34 | B | D | D | C |
| 35 | C | D | D | C |
| 36 | B | D | D | D |
| 37 | C | C | C | C |
| 38 | A | B | B | B |
| 39 | D | D | D | B |
| 40 | C | C | C | C |
| 41 | A | B | A | C |
| 42 | B | D | D | C |
| 43 | A | C | D | C |
| 44 | D | D | D | C |
| 45 | D | D | D | D |
| 46 | D | D | D | C |
| 47 | C | D | D | D |
| 48 | B | D | C | D |

### Example 51

### In Vitro Antibacterial Assays of β-Lactamase Inhibition

To determine the ability of test compounds to potentiate the inhibition of the growth of bacterial strains producing beta-lactamase enzymes, classic cell based screening assays were employed. Four bacteria strains producing beta-lactamase enzymes were used: K. pneumoniae expressing the Class A Extended Spectrum Beta-Lactamase (ESBL) CTX-M-15, E. coli expressing the Class A ESBL SHV-5, E. cloacae expressing the Class C P99+, and K. pneumoniae expressing the Class A carbapenemase KPC-2. In order to evaluate the ability of test compounds to inhibit beta-lactamase activity, Applicants used a modification of the broth microdilution assay. The assay was conducted in Cation Adjusted Mueller Hinton Broth (CAMHB, BD #212322, BD Diagnostic Systems, Sparks, Md.). Bacteria strains were grown for 3-5 hours in CAMBH broth. All four strains were grown in presence of 50 μg/mL ampicillin to ensure resistance is maintained. In the meantime, test compounds were diluted in DMSO to a 0.1 mg/mL stock. The compounds were added to a microtiter plate and were diluted in 2-fold serial dilutions in CAMHB in a final concentration range of 8 μg/mL to 0.015 μg/ml. An overlay of CAMHB containing a cephalosporin was added to the compounds at a final static concentration of 8 μg/ml. Ceftazidime (CAZ, Sigma# C3809-1G, Sigma-Aldrich, St. Louis, Mo.) was used as the partner antibiotic for K. pneumoniae expressing Ambler Class A ESBL CTX-M-15 (MIC alone=128 μg/ml), E. coli expressing Class A ESBL SHV-5 (MIC alone>1024 μg/mL), K. pneumoniae expressing Ambler Class A carbapenemase KPC-2 (MIC alone=32 μg/mL), and E. cloacae expressing Class C P99+AmpC (MIC alone=128 μg/mL). Titration of test compounds with MIC readout indicates the concentration of test article needed to sufficiently inhibit beta lactamase enzyme activity and protect the intrinsic antibacterial activity of the cephalosporin. Each of these compound plates are made in quadruplicate, one for each bacteria strain. In addition to the titration of test compounds the MICs of a panel of cephalosporins is also tested to ensure the strains are behaving consistently from test to test. Once the test compound and cephalosporin are added the plates can be inoculated. Inocula are conducted according to CLSI broth microdilution method. After inoculation the plates are incubated for 16-20 hours at 37° C. then the Minimal Inhibitory Concentration (MIC) of the test compound is determined visually.

Using the methodology described above, examples of the current invention were evaluated for their ability to inhibit the growth of β-lactamase producing bacteria in the presence of a β-lactam antibiotic. Representative results are shown in Table 3 where A represents an MIC>8 μg/mL, B represents an MIC between 1 and 8 μg/mL, and C represents an MIC of <1 μg/mL. NT=Not Tested.

TABLE 3
Broad spectrum inhibition of bacterial growth. MIC of example
compounds of the invention in the presence of a fixed amount
(8 μg/mL) of Ceftazidime β-lactam antibiotic.

| | E. coli | | | |
|---|---|---|---|---|
| | SHV-5 + | K. pneumoniae | E. cloacae P99 | K. pneumoniae |
| | 8 μg/mL | CTX-M-15 + 8 | AmpC + 8 | KPC-2 + |
| Example | CAZ | μg/mL CAZ | μg/mL CAZ | 8 μg/mL CAZ |
| 1 | C | C | B | C |
| 2 | B | B | A | NT |
| 3 | B | C | B | C |
| 4 | C | C | C | C |
| 5 | B | C | C | C |
| 6 | B | C | B | C |
| 7 | C | C | B | C |
| 8 | B | C | B | C |
| 9 | C | C | B | C |
| 10 | C | C | C | C |
| 11 | B | C | C | C |
| 12 | B | C | B | C |
| 13 | B | C | C | C |
| 14 | B | C | C | C |
| 15 | B | C | C | C |
| 16 | B | C | C | C |
| 17 | A | C | C | B |
| 18 | B | C | B | B |
| 19 | A | C | B | C |
| 20 | B | C | C | C |
| 21 | C | C | B | C |
| 22 | B | C | B | C |
| 23 | C | C | C | C |
| 24 | B | C | B | C |
| 25 | B | C | B | C |
| 26 | B | C | C | C |
| 27 | B | B | B | C |
| 28 | B | C | B | C |
| 29 | C | C | B | C |
| 30 | B | B | B | C |
| 31 | B | C | B | C |
| 32 | B | B | B | C |
| 33 | B | B | B | C |
| 34 | A | B | B | C |
| 35 | B | B | B | C |
| 36 | B | C | C | C |
| 37 | B | C | C | C |
| 38 | A | A | B | C |
| 39 | C | C | C | C |
| 40 | B | C | B | C |
| 41 | A | A | A | C |
| 42 | B | B | C | C |
| 43 | A | A | B | A |
| 44 | A | A | B | B |
| 45 | B | B | B | C |
| 46 | B | C | C | C |
| 47 | A | A | B | B |
| 48 | A | B | B | C |

1. A compound of the formula: 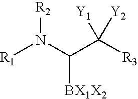
wherein R₁ is —C(O)R₄; —C(O)NR₄R₅; —C(O)OR₄; —S(O)₂R₄, —C(═NR₄R₅)R₄, —C(═NR₄R₅)NR₄R₅, hydrogen, or is selected from the group consisting of:
(a) aryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido,
(b) heteroaryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido, and
(c) heterocyclic group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido; R₂ is hydrogen, or is selected from the group consisting of:
(a) C1-C6 alkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the C1-C6 carbons comprise part of said oxyimino group, sulfido, and sulfoxido,
(b) C3-C7 cycloalkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the cycloalkyl group other than the one attached to the rest of the molecule comprise part of said oxyimino group, sulfido, and sulfoxido,
(c) aryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido,
(d) heteroaryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido, and
(e) heterocyclic group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, sulfido, and sulfoxido;
R₃ is an aryl or heteroaryl group substituted with from 1 to 4 substituents wherein one of the substituents is a hydroxyl or amino group located at the 2 position relative to the group containing Y₁ and Y₂, and wherein the remaining substituents are selected from the group consisting of hydroxyl, alkyl, cycloalkyl, alkoxy, alkenyl, alkynyl, amino, aminocarbonyl, carbonyl, aminosulfonyl, alkylaryl, aryl, aryloxy, carboxyl, cyano, guanidino, halogen, heteroaryl, heterocyclyl, sulfido, sulfonyl, sulfoxido, sulfonic acid, sulfate, and thiol;
R₄ is selected from the group consisting of:
(a) C1-C10 alkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the C1-C10 carbons comprise part of said oxyimino group, sulfido, and sulfoxido,
(b) C3-C10 cycloalkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the cycloalkyl group other than the one attached to the rest of the molecule comprise part of said oxyimino group, sulfido, and sulfoxido,
(c) aryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido,
(d) heteroaryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido, and
(e) heterocyclic group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, sulfido, and sulfoxido;
R₅ is hydrogen or is selected from the group consisting of:
(a) C1-C6 alkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the C1-C10 carbons comprise part of said oxyimino group, sulfido, and sulfoxido,
(b) C3-C7 cycloalkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the cycloalkyl group other than the one attached to the rest of the molecule comprise part of said oxyimino group, sulfido, and sulfoxido,
(c) aryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido,
(d) heteroaryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido, and
(e) heterocyclic group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, sulfido, and sulfoxido;
X₁ and X₂ are independently hydroxyl, halogen, NR₄R₅, C1-C6 alkoxy, or when taken together X₁ and X₂ form a cyclic boron ester where said chain or ring contains from 2 to 20 carbon atoms and, optionally, 1-3 heteroatoms which can be O, N, or S, or when taken together X₁ and X₂ form a cyclic boron amide where said chain or ring contains from 2 to 20 carbon atoms and, optionally, 1-3 heteroatoms which can be O, N, or S, or when taken together X₁ and X₂ form a cyclic boron amide-ester where said chain contains from 2-20 carbon atoms and, optionally, 1-3 heteroatoms which can be O, N, or S, or X₁ and R₁ together form a cyclic ring where said ring contains 2 to 10 carbon atoms and, optionally, 1-3 heteroatoms which can be O, N, or S, and X₂ is hydroxyl, halogen, NR₄R₅, C1-C6 alkoxy, or X₁ and R₃ together form a cyclic ring where said ring contains 3 to 10 carbon atoms and, optionally, 1-3 heteroatoms which can be O, N, or S, and X₂ is hydroxyl, halogen, NR₄R₅, or C1-C6 alkoxy;
Y₁ and Y₂ are independently hydrogen, alkyl, cycloalkyl, alkoxy, alkenyl, alkynyl, amino, aminosulfonyl, aminocarbonyl, carbonyl, alkylaryl, aryl, aryloxy, carboxyl, cyano, halogen, heteroaryl, heteroaryloxy, heterocyclyl, sulfido, sulfonyl, or sulfoxido, or taken together Y₁ and Y₂ form a cyclic structure containing from 3-12 carbon atoms and, optionally, 1-3 heteroatoms which can be O, N, or S;
or a salt thereof;
provided that, when R₁ is —C(O)R₄, R₂ is hydrogen, R₃ is a phenyl group having two substituents consisting of a hydroxyl at the 2-position and a carboxylic acid at the 3-position relative to the group containing Y₁ and Y₂, X₁ and X₂ are hydroxyl or X₁ is hydroxyl and X₂ is replaced by the ortho-hydroxyl oxygen of R₃ such that a 6-membered ring is formed, and Y₁ and Y₂ are hydrogen, R₄ is not unsubstituted C1 alkyl.

2. The compound of claim 1, wherein R₁ is —C(O)R₄; R₂ is hydrogen; R₃ is an aryl or heteroaryl group substituted with from 2 to 4 substituents wherein one of the substituents is a hydroxyl or amino group located at the 2-position relative to the group containing Y₁ and Y₂, and a second substituent is a carboxylic acid group and wherein the remaining substituents are selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido;
R₄ is selected from the group consisting of:
(a) C1-C10 alkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the C1-C10 carbons comprise part of said oxyimino group, sulfido, and sulfoxido,
(b) C3-C10 cycloalkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the cycloalkyl group other than the one attached to the rest of the molecule comprise part of said oxyimino group, sulfido, and sulfoxido,
(c) aryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido,
(d) heteroaryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido, and
(e) heterocyclic group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, sulfido, and sulfoxido;
X₁ and X₂ are hydroxyl, or when taken together X₁ and X₂ form a cyclic boron ester where said chain or ring contains from 2 to 20 carbon atoms and, optionally, 1-3 heteroatoms which can be O, N, or S, or X₁ and R₁ together form a cyclic ring where said ring contains 2 to 10 carbon atoms and, optionally, 1-3 heteroatoms which can be O, N, or S, and X₂ is hydroxyl, or X₁ and R₃ together form a cyclic ring where said ring contains 3 to 10 carbon atoms and, optionally, 1-3 heteroatoms which can be O, N, or S and X₂ is hydroxyl;
Y₁ and Y₂ are independently hydrogen, alkyl, cycloalkyl, alkoxy, alkenyl, alkynyl, amino, aminosulfonyl, aminocarbonyl, carbonyl, alkylaryl, aryl, aryloxy, carboxyl, cyano, halogen, heteroaryl, heteroaryloxy, heterocyclyl, sulfido, sulfonyl, or sulfoxido;
or a salt thereof;
provided that, when R₃ is a phenyl group having two substituents consisting of a hydroxyl at the 2-position and a carboxylic acid at the 3-position relative to the group containing Y₁ and Y₂, X₁ and X₂ are hydroxyl or X₁ is hydroxyl and X₂ is replaced by the ortho-hydroxyl oxygen of R₃ such that a 6-membered ring is formed, and Y₁ and Y₂ are hydrogen, R₄ is not unsubstituted C1 alkyl.

3. The compound of claim 2, wherein R₁ is —C(O)R₄; R₂ is hydrogen; R₃ is an aryl group having a hydroxyl at the 2-position and a carboxylic acid at the 3-position relative to the group containing Y₁ and Y₂; R₄ is C1-C10 alkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the C1-C10 carbons comprise part of said oxyimino group, sulfido, and sulfoxido; X₁ and X₂ are hydroxyl, or X₁ is hydroxyl and X₂ is replaced by the ortho-hydroxyl oxygen of R₃ such that a 6-membered ring is formed; and Y₁ and Y₂ are hydrogen;
or a salt thereof;
provided that, when R₃ is a phenyl group having two substituents consisting of a hydroxyl at the 2-position and a carboxylic acid at the 3-position relative to the group containing Y₁ and Y₂, and X₁ and X₂ are hydroxyl or X₁ is hydroxyl and X₂ is replaced by the ortho-hydroxyl oxygen of R₃ such that a 6-membered ring is formed, R₄ is not unsubstituted C1 alkyl.

4. The compound of claim 2, wherein R₁ is —C(O)R₄; R₂ is hydrogen; R₃ is an aryl group having a hydroxyl at the 2-position and a carboxylic acid at the 3-position relative to the group containing Y₁ and Y₂; R₄ is C3-C10 cycloalkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the cycloalkyl group other than the one attached to the rest of the molecule comprise part of said oxyimino group, sulfido, and sulfoxido; X₁ and X₂ are hydroxyl, or X₁ is hydroxyl and X₂ is replaced by the ortho-hydroxyl oxygen of R₃ such that a 6-membered ring is formed; and Y₁ and Y₂ are hydrogen; or a salt thereof.

5. The compound of claim 2, wherein R₁ is —C(O)R₄; R₂ is hydrogen; R₃ is an aryl group having a hydroxyl at the 2-position and a carboxylic acid at the 3-position relative to the group containing Y₁ and Y₂; R₄ is aryl or heteroaryl substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido; X₁ and X₂ are hydroxyl, or X₁ is hydroxyl and X₂ is replaced by the ortho-hydroxyl oxygen of R₃ such that a 6-membered ring is formed; and Y₁ and Y₂ are hydrogen; or a salt thereof.

6. The compound of claim 2, wherein R₁ is —C(O)R₄; R₂ is hydrogen; R₃ is an aryl group having a hydroxyl at the 2-position and a carboxylic acid at the 3-position relative to the group containing Y₁ and Y₂; R₄ is a heterocycle substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the cycloalkyl group other than the one attached to the rest of the molecule comprise part of said oxyimino group, sulfido, and sulfoxido; X₁ and X₂ are hydroxyl, or X₁ is hydroxyl and X₂ is replaced by the ortho-hydroxyl oxygen of R₃ such that a 6-membered ring is formed; and Y₁ and Y₂ are hydrogen; or a salt thereof.

7. The compound of claim 2, wherein the compound is 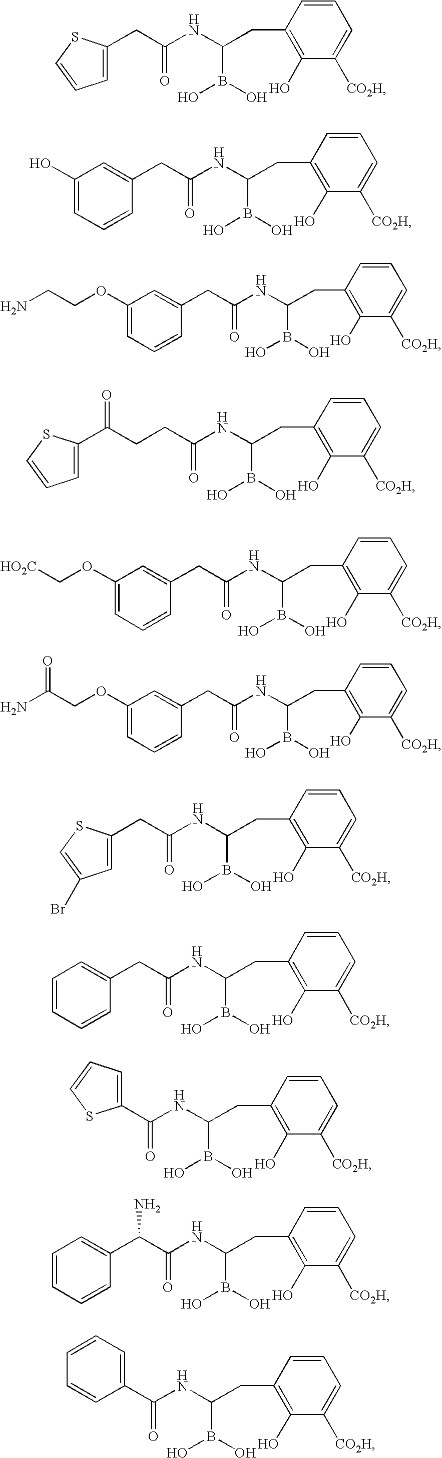 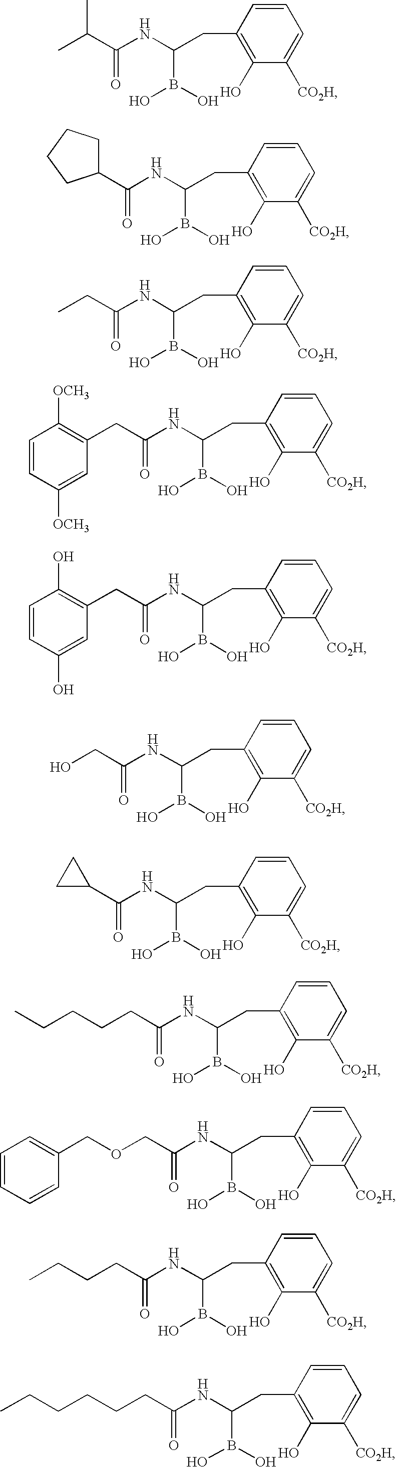 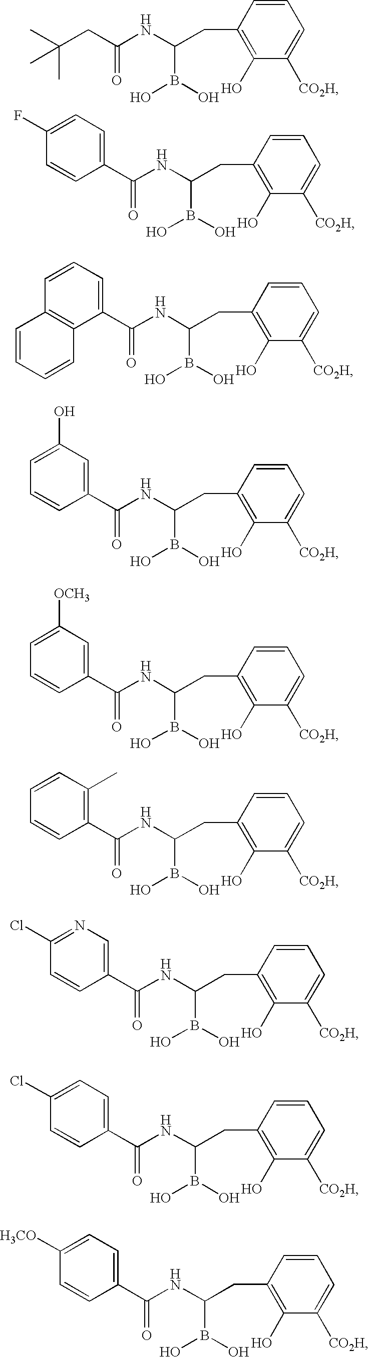 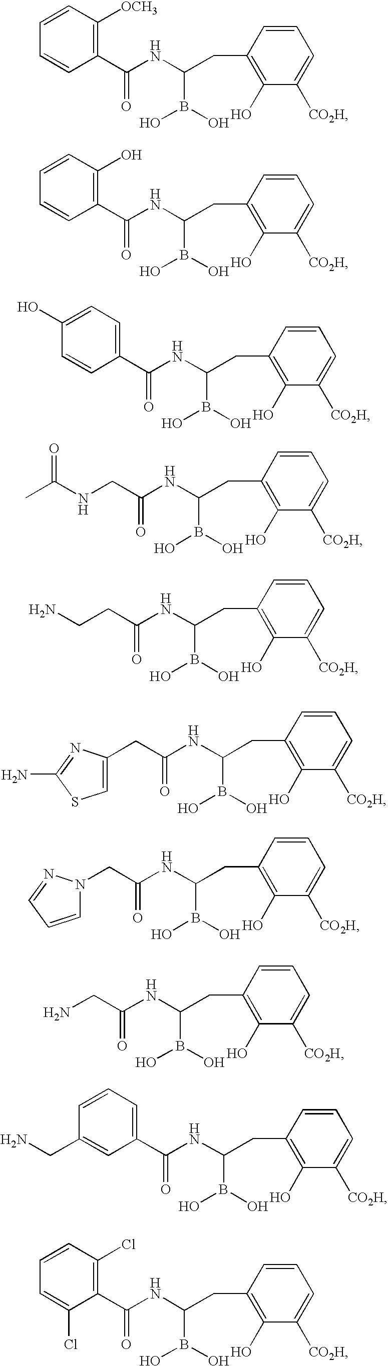 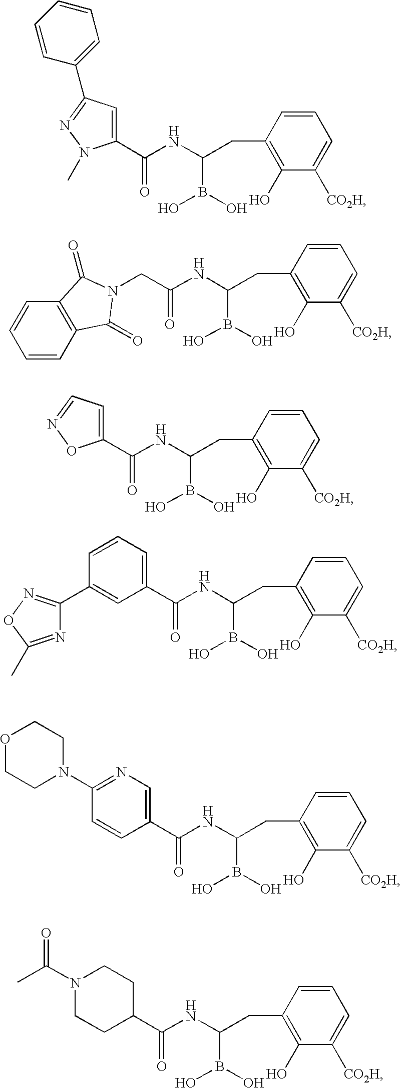
or a salt thereof.

8. A pharmaceutical composition comprising:
(a) one or more compounds of claim 1;
(b) one or more β-lactam antibiotics; and
(c) one or more pharmaceutically acceptable carriers.

9. The pharmaceutical composition of claim 8, wherein the β-lactam antibiotic is a penicillin, cephalosporin, carbapenem, monobactam, bridged monobactam, or combination thereof.

10. The pharmaceutical composition of claim 9, wherein the penicillin is benzathine penicillin, benzylpenicillin, phenoxymethylpenicillin, procaine penicillin, oxacillin, methicillin, dicloxacillin, flucloxacillin, temocillin, amoxicillin, ampicillin, co-amoxiclav, azlocillin, carbenicillin, ticarcillin, mezlocillin, piperacillin, apalcillin, hetacillin, bacampicillin, sulbenicillin, mecicilam, pevmecillinam, ciclacillin, talapicillin, aspoxicillin, cloxacillin, nafcillin, pivampicillin, or a combination thereof.

11. The pharmaceutical composition of claim 9, wherein the cephalosporin is cephalothin, cephaloridin, cefaclor, cefadroxil, cefamandole, cefazolin, cephalexin, cephradine, ceftizoxime, cefoxitin, cephacetril, cefotiam, cefotaxime, cefsulodin, cefoperazone, ceftizoxime, cefinenoxime, cefinetazole, cephaloglycin, cefonicid, cefodizime, cefpirome, ceftazidime, ceftriaxone, cefpiramide, cefbuperazone, cefozopran, cefepim, cefoselis, cefluprenam, cefuzonam, cefpimizole, cefclidin, cefixime, ceftibuten, cefdinir, cefpodoxime axetil, cefpodoxime proxetil, cefteram pivoxil, cefetamet pivoxil, cefcapene pivoxil, cefditoren pivoxil, cefuroxime, cefuroxime axetil, loracarbacef, latamoxef, ceftobiprole, ceftaroline, or a combination thereof.

12-13. (canceled)

14. The pharmaceutical composition of claim 9, wherein the carbapenem is imipenem, meropenem, ertapenem, faropenem, doripenem, biapenem, panipenem, PZ-601, ME1036 or a combination thereof.

15-16. (canceled)

17. The pharmaceutical composition of claim 9, wherein the monobactam is aztreonam, carumonam, BAL30072, or a combination thereof.

18. A pharmaceutical composition comprising:
(a) one or more compounds of claim 1; and
(b) one or more pharmaceutically acceptable carriers.

19. The pharmaceutical composition of claim 8, comprising more than one beta-lactam antibiotic.

20. A method of treating a bacterial infection in a mammal comprising administering to a mammal in need thereof:
(i) an effective amount of a compound having the formula: 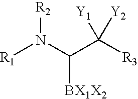
wherein R₁ is —C(O)R₄; —C(O)NR₄R₅; —C(O)OR₄; —S(O)₂R₄, —C(═NR₄R₅)R₄, —C(═NR₄R₅)NR₄R₅, hydrogen, or is selected from the group consisting of:
(a) aryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido,
(b) heteroaryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido, and
(c) heterocyclic group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido;
R₂ is hydrogen, or is selected from the group consisting of:
(a) C1-C6 alkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the C1-C6 carbons comprise part of said oxyimino group, sulfido, and sulfoxido,
(b) C3-C7 cycloalkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the cycloalkyl group other than the one attached to the rest of the molecule comprise part of said oxyimino group, sulfido, and sulfoxido,
(c) aryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido,
(d) heteroaryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido, and
(e) heterocyclic group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, sulfido, and sulfoxido;
R₃ is an aryl or heteroaryl group substituted with from 1 to 4 substituents wherein one of the substituents is a hydroxyl or amino group located at the 2 position relative to the group containing Y₁ and Y₂, and wherein the remaining substituents are selected from the group consisting of hydroxyl, alkyl, cycloalkyl, alkoxy, alkenyl, alkynyl, amino, aminocarbonyl, carbonyl, aminosulfonyl, alkylaryl, aryl, aryloxy, carboxyl, cyano, guanidino, halogen, heteroaryl, heterocyclyl, sulfido, sulfonyl, sulfoxido, sulfonic acid, sulfate, and thiol;
R₄ is selected from the group consisting of:
(a) C1-C10 alkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the C1-C10 carbons comprise part of said oxyimino group, sulfido, and sulfoxido,
(b) C3-C10 cycloalkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the cycloalkyl group other than the one attached to the rest of the molecule comprise part of said oxyimino group, sulfido, and sulfoxido,
(c) aryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido,
(d) heteroaryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido, and
(e) heterocyclic group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, sulfido, and sulfoxido;
R₅ is hydrogen or is selected from the group consisting of:
(a) C1-C6 alkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the C1-C10 carbons comprise part of said oxyimino group, sulfido, and sulfoxido,
(b) C3-C7 cycloalkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the cycloalkyl group other than the one attached to the rest of the molecule comprise part of said oxyimino group, sulfido, and sulfoxido,
(c) aryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido,
(d) heteroaryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido, and
(e) heterocyclic group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, sulfido, and sulfoxido;
X₁ and X₂ are independently hydroxyl, halogen, NR₄R₅, C1-C6 alkoxy, or when taken together X₁ and X₂ form a cyclic boron ester where said chain or ring contains from 2 to 20 carbon atoms and, optionally, 1-3 heteroatoms which can be O, N, or S, or when taken together X₁ and X₂ form a cyclic boron amide where said chain or ring contains from 2 to 20 carbon atoms and, optionally, 1-3 heteroatoms which can be O, N, or S, or when taken together X₁ and X₂ form a cyclic boron amide-ester where said chain contains from 2-20 carbon atoms and, optionally, 1-3 heteroatoms which can be O, N, or S, or X₁ and R₁ together form a cyclic ring where said ring contains 2 to 10 carbon atoms and, optionally, 1-3 heteroatoms which can be O, N, or S, and X₂ is hydroxyl, halogen, NR₄R₅, C1-C6 alkoxy, or X₁ and R₃ together form a cyclic ring where said ring contains 3 to 10 carbon atoms and, optionally, 1-3 heteroatoms which can be O, N, or S, and X₂ is hydroxyl, halogen, NR₄R₅, or C1-C6 alkoxy;
Y₁ and Y₂ are independently hydrogen, alkyl, cycloalkyl, alkoxy, alkenyl, alkynyl, amino, aminosulfonyl, aminocarbonyl, carbonyl, alkylaryl, aryl, aryloxy, carboxyl, cyano, halogen, heteroaryl, heteroaryloxy, heterocyclyl, sulfido, sulfonyl, or sulfoxido, or taken together Y₁ and Y₂ form a cyclic structure containing from 3-12 carbon atoms and, optionally, 1-3 heteroatoms which can be O, N, or S;
or a salt thereof; and
(ii) an effective amount of a β-lactam antibiotic.

21. The method of claim 20, wherein the mammal is a human.

22. A method of treating a bacterial infection in a mammal comprising administering to a mammal in need thereof an effective amount of a compound having the formula: 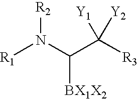
wherein R₁ is —C(O)R₄; —C(O)NR₄R₅; —C(O)OR₄; —S(O)₂R₄, —C(═NR₄R₅)R₄, —C(═NR₄R₅)NR₄R₅, hydrogen, or is selected from the group consisting of:
(a) aryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido,
(b) heteroaryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido, and
(c) heterocyclic group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido;
R₂ is hydrogen, or is selected from the group consisting of:
(a) C1-C6 alkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the C1-C6 carbons comprise part of said oxyimino group, sulfido, and sulfoxido,
(b) C3-C7 cycloalkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the cycloalkyl group other than the one attached to the rest of the molecule comprise part of said oxyimino group, sulfido, and sulfoxido,
(c) aryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido,
(d) heteroaryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido, and
(e) heterocyclic group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, sulfido, and sulfoxido;
R₃ is an aryl or heteroaryl group substituted with from 1 to 4 substituents wherein one of the substituents is a hydroxyl or amino group located at the 2 position relative to the group containing Y₁ and Y₂, and wherein the remaining substituents are selected from the group consisting of hydroxyl, alkyl, cycloalkyl, alkoxy, alkenyl, alkynyl, amino, aminocarbonyl, carbonyl, aminosulfonyl, alkylaryl, aryl, aryloxy, carboxyl, cyano, guanidino, halogen, heteroaryl, heterocyclyl, sulfido, sulfonyl, sulfoxido, sulfonic acid, sulfate, and thiol;
R₄ is selected from the group consisting of:
(a) C1-C10 alkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the C1-C10 carbons comprise part of said oxyimino group, sulfido, and sulfoxido,
(b) C3-C10 cycloalkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the cycloalkyl group other than the one attached to the rest of the molecule comprise part of said oxyimino group, sulfido, and sulfoxido,
(c) aryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido,
(d) heteroaryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido, and
(e) heterocyclic group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, sulfido, and sulfoxido;
R₅ is hydrogen or is selected from the group consisting of:
(a) C1-C6 alkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the C1-C10 carbons comprise part of said oxyimino group, sulfido, and sulfoxido,
(b) C3-C7 cycloalkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the cycloalkyl group other than the one attached to the rest of the molecule comprise part of said oxyimino group, sulfido, and sulfoxido,
(c) aryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido,
(d) heteroaryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido, and
(e) heterocyclic group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, sulfido, and sulfoxido;
X₁ and X₂ are independently hydroxyl, halogen, NR₄R₅, C1-C6 alkoxy, or when taken together X₁ and X₂ form a cyclic boron ester where said chain or ring contains from 2 to 20 carbon atoms and, optionally, 1-3 heteroatoms which can be O, N, or S, or when taken together X₁ and X₂ form a cyclic boron amide where said chain or ring contains from 2 to 20 carbon atoms and, optionally, 1-3 heteroatoms which can be O, N, or S, or when taken together X₁ and X₂ form a cyclic boron amide-ester where said chain contains from 2-20 carbon atoms and, optionally, 1-3 heteroatoms which can be O, N, or S, or X₁ and R₁ together form a cyclic ring where said ring contains 2 to 10 carbon atoms and, optionally, 1-3 heteroatoms which can be O, N, or S, and X₂ is hydroxyl, halogen, NR₄R₅, C1-C6 alkoxy, or X₁ and R₃ together form a cyclic ring where said ring contains 3 to 10 carbon atoms and, optionally, 1-3 heteroatoms which can be O, N, or S, and X₂ is hydroxyl, halogen, NR₄R₅, or C1-C6 alkoxy;
Y₁ and Y₂ are independently hydrogen, alkyl, cycloalkyl, alkoxy, alkenyl, alkynyl, amino, aminosulfonyl, aminocarbonyl, carbonyl, alkylaryl, aryl, aryloxy, carboxyl, cyano, halogen, heteroaryl, heteroaryloxy, heterocyclyl, sulfido, sulfonyl, or sulfoxido, or taken together Y₁ and Y₂ form a cyclic structure containing from 3-12 carbon atoms and, optionally, 1-3 heteroatoms which can be O, N, or S;
or a salt thereof.

23-28. (canceled)
